(19) United States
(12) Patent Application Publication
(10) Pub. No.: US 2016/0326220 A1
(43) Pub. Date: Nov. 10, 2016

(54) SECRETION AND FUNCTIONAL DISPLAY OF CHIMERIC POLYPEPTIDES

(71) Applicants: VIB VZW, Gent (BE); VRIJE UNIVERSITEIT BRUSSEL, Brussel (BE)

(72) Inventors: Han Remaut, Roosbeek (BE); Nani Van Gerven, Huizingen (BE)

(21) Appl. No.: 15/108,256

(22) PCT Filed: Dec. 24, 2014
(86) PCT No.: PCT/EP2014/079319

§ 371 Date: Jun. 24, 2016

(30) Foreign Application Priority Data
Dec. 24, 2013 (EP) 13199513.6

(51) Int. Cl. C07K 14/245; C12P 21/02; C12N 9/16; C07K 14/435; C12N 9/22; C12N 9/86; C12N 15/10; C07K 16/18

(52) U.S. Cl. CPC C07K 14/245; C12N 15/1037; C12P 21/02; C07K 16/18; C07K 14/43595; C12N 9/22; C12N 9/86; C12N 9/16; C12Y 301/00; C12Y 305/02006; C07K 2319/02; C07K 2317/22; C07K 2319/10; C07K 2319/60; C07K 2319/61; C07K 2319/30; C07K 2317/14

(57) ABSTRACT

This disclosure relates to the display of proteins and peptides on cellular or non-biotic surfaces in the form of multivalent filamentous polymers. In particular, the disclosure provides for tools and methods for the secretion and functional display of chimeric polypeptides on the surface of cells, in particular, bacterial cells, as well as on foreign substrates, both biological and synthetic. Further envisaged are biotechnological applications using the same.

### CROSS-REFERENCE TO RELATED APPLICATIONS

This application is a national phase entry under 35 U.S.C. §371 of International Patent Application PCT/EP2014/079319, filed Dec. 24, 2014, designating the United States of America and published in English as International Patent Publication WO 2015/097289 A1 on Jul. 2, 2015, which claims the benefit under Article 8 of the Patent Cooperation Treaty to European Patent Application Serial No. 13199513.6, filed Dec. 24, 2013.

### STATEMENT ACCORDING TO 37 C.F.R. §1.821(c) or (e)—SEQUENCE LISTING SUBMITTED

Pursuant to 37 C.F.R. §1.821(c) or (e), a file containing an electronic version of the Sequence Listing has been submitted concomitant with this application, the contents of which are hereby incorporated by reference. One file titled “V484_ST25” that is 42 KB and created on Jun. 14, 2016, is submitted electronically.

### TECHNICAL FIELD

This application relates to the display of proteins and peptides on cellular or non-biotic surfaces in the form of multivalent filamentous polymers. In particular, the disclosure provides for tools and methods for the secretion and functional display of chimeric polypeptides on the surface of cells, in particular, bacterial cells, as well as on foreign substrates, both biological and synthetic. Further envisaged are biotechnological applications using the same.

### BACKGROUND

A wide variety of biotechnological applications seek the immobilization of polypeptides on biological or synthetic surfaces.

The display of polypeptides on a cellular surface has been a subject of investigation for several years. Cellular surface display bears considerable advantages for numerous biotechnical applications including recombinant vaccines, combinatorial library screening, reagents for diagnostics, and whole-cell biocatalysts and biosorbents (Lee et al., 2003; Wernerus and Stahl, 2004). An attractive way to present proteins (or segments thereof) on the bacterial surface is to graft them into permissible positions on naturally occurring surface proteins. The first papers to describe microbial surface display fall within the field of vaccine development using the E. coli outer membrane proteins LamB (Charbit et al., 1986), OmpA (Ruppert et al., 1994) and PhoE (Agterberg and Tommassen, 1991) to display short gene fragments. Since then, a variety of anchoring motifs have been developed for the display of heterologous peptides and proteins, including S-layer proteins, lipoproteins, autotransporters and subunits of surface appendages (Samuelson et al., 2002; Lee et al., 2003). Among these various mechanisms, fibrillar structures such as flagella, pili and curli are especially attractive candidates because of their natural function and/or highly organized multi-subunit features. Both the major and minor structural subunits of flagella and pili were employed to transport passenger proteins onto the cell surface (reviewed in: Van Gerven et al., 2011).

While shown successful for several, not all proteins can be efficiently exposed on the bacterial surface using multi-subunit fibers. One of the problems usually encountered with flagellar or fimbrial display systems is the limited size of heterologous grafts that can be displayed without causing detrimental effects on the structure and/or function of the carrier protein. For pili of the chaperone-usher pathway (also referred to as fimbriae), the upper size limit seems to be relatively low, being 34 AA and 52 AA for, respectively, the major and minor tip subunits (Samuelson et al., 2002). Studies addressing the mechanisms of curli display are sporadic, with only short sequences being displayed (White et al., 1999; White et al., 2000; Huang et al., 2009; Meng et al., 2010). In these studies, regions within the major Salmonella curli subunit, AgfA, were replaced by different T-cell epitopes, as was also described in a patent application published as WO2008/124646.

High-density surface expression of recombinant proteins is a prerequisite for successfully using cellular surface display in several areas of biotechnological applications, including the construction of oral live vaccines and whole-cell biocatalysts in the fields of pharmaceutical, fine chemical, bioconversion, waste treatment and agrochemical production. An ideal display system should combine the ability to accommodate large inserts with a high copy number and a broad host range.

In addition, a range of biotechnological applications make use of the coating or activation of synthetic surfaces with polypeptides. Usually, this coating occurs through the covalent coupling or through (affinity-based) adsorption of the polypeptides to the desired material. Both approaches can have a number of problems or disadvantages: (1) for both strategies, the coating procedure is rather non-specific, requiring that the polypeptide samples are of a high degree of purity prior to the coating procedure in order to avoid the inclusion of contaminants, which would dilute the density of the desired polypeptide and which may add undesired properties to the coating. This need for a purification step often adds to the production expense; (2) the chemical composition of the material or the conditions required for covalent or adsorption-based coating of polypeptides can lead to the loss of the active conformation of the polypeptides; (3) the chemical build-up of the materials or the conditions required to allow polypeptide adsorption may not be compatible with downstream usage; and (4) adsorption-based coatings can lose polypeptides to the soluble fraction, leading to a depletion of the polypeptide density over time.

Therefore, a system that couples the bio-production of the desired polypeptides with a self-assembling property that leads to the formation of thread-like polymers onto a synthetic surface and that displays the polypeptide in an active conformation would alleviate a number of these disadvantages.

### BRIEF SUMMARY

This disclosure is based on the unexpected finding that fusion of intact proteins (“passenger polypeptide” as defined further herein) to carrier proteins derived from bacterial fiber subunit proteins from the curli family (“carrier polypeptide” as defined further herein) is feasible and can successfully be used for the display of correctly folded and active proteins into filamentous threads, either on the bacterial cell surface or on foreign (synthetic) substrates. Bacterial fiber subunit proteins of the curli family can act as a versatile scaffold for secretion and surface display of heterologous proteinaceous inserts, which offers a number of advantages. First of all, the carriage of passenger proteins does not interfere with the correct secretion of the fiber subunit to the extracellular environment by the producing bacterium. Second, the fiber subunit carrier protein is competent for self-assembly into curli-like fibers and can accommodate and display entire and functionally active proteins into the fibers. Third, fibers are a high-valency display system, as the high copy number of the fiber subunit does not seem to be significantly affected by most foreign inserts. As a comparison, the major structural proteins of various fimbriae can only contain modest-sized inserts (in the 10-30 amino acid range) without detrimental effects on organelle structure and surface display. The minor adhesin component at the tip seems to be more accommodating but is still only capable of displaying peptides of around 100 amino acids (Pallesen et al., 1995), and results in single-copy display at the tip of the organelle. Thus, display using curli-like fibers is a promising tool for various approaches in biotechnology and biomedicine, demonstrating that, in addition to the export of peptides, proteins retaining their activity can be displayed successfully into the amyloid fibers on the bacterial cell surface or on foreign substrates, both biologically and synthetically.

Typically, curli fiber subunit proteins have strongly conserved motifs. Another unexpected finding of this disclosure is that the presence of a particularly conserved motif in the carrier protein seems to be sufficient for secretion and fiber formation, as well as for the carriage of passenger proteins and the display of correctly folded functional proteins into the fibers. This is particularly advantageous since it allows designing a fusion protein of choice in view of the desired characteristics of either the fiber and/or the display of the heterologous inserts.

Another unexpected finding of this disclosure is that a bacterial Type VIII secretion system is amenable for the transport and secretion of correctly folded and active proteins outside a bacterial cell.

Another unexpected finding is that curli subunits can be secreted from a non-native host, including a Gram-positive bacterium, and that these secreted subunits are competent to form extracellular curli fibers. Production of curli fibers by a non-native host bacterium includes the secretion and assembly of heterologous proteins and peptides fused to the curli subunit CsgA or to defined peptide fragments derived thereof.

One aspect of the present application relates to a method of producing a functionalized fiber, the method comprising the steps of:
a) providing a host cell that is genetically engineered to express a chimeric polypeptide comprising:
   i. a carrier polypeptide comprising an amino acid sequence V/I/L-X-Q-X-G-X-X-N/Q-X-A/V/I/L-X-X-X-Q (SEQ ID NO: 32), wherein X means any amino acid,
   ii. a passenger polypeptide of 50 amino acids or more, and
   iii. optionally, a linker that couples a) to b),
b) culturing the host cell of a) under suitable conditions to express the chimeric polypeptide, and
c) allowing the chimeric polypeptide to polymerize into a fiber, whereby the passenger polypeptide is displayed as a functionally active polypeptide.

In one embodiment of the above method, step c) occurs on or near the extracellular surface of the same or another host cell. In another embodiment, step c) occurs on or near an artificial surface. In yet another embodiment, step c) occurs in solution.

In one embodiment of the above method, the expressed chimeric polypeptide is secreted.

Also, the above method may further comprise the step of:
d) isolating the expressed chimeric polypeptide from the cell before step c).

Preferably, for the above method, the passenger polypeptide of the chimeric polypeptide is maintained as a functionally active polypeptide after secretion or isolation.

In a particular embodiment of the above method, the host cell is a bacterial host cell, in particular a Gram-negative bacterial host cell, or a Gram-positive bacterial host cell.

In yet another embodiment of the above method, the host cell expresses, either endogenously or exogenously, a nucleic acid sequence encoding CsgG, and at least one nucleic acid sequence encoding one or more of CsgB, CsgC, CsgE, CsgF, or variants or fragments of any thereof.

In a particular embodiment of the above method, the recombinant nucleic acid molecule encoding the chimeric polypeptide and the one or more nucleic acid sequences are expressed simultaneously.

According to a preferred embodiment of the above method, the carrier polypeptide of the chimeric polypeptide has the following structure: (Y₂ᵢ₋₁-Xᵢ-Y₂ᵢ)ₙ, wherein:
a) n is an integer from 1 to 20 and i increases from 1 to n with each repeat;
b) each Xᵢ corresponds to the amino acid sequence V/I/L-X-Q-X-G-X-X-N/Q-X-A/V/I/L-X-X-X-Q (SEQ ID NO: 32), wherein X means any amino acid; and
c) each Y₂ᵢ₋₁ and Y₂ᵢ are independently selected from 0 to 20 contiguous amino acids, wherein the total length of each Y₂ᵢ₋₁-Xᵢ-Y₂ᵢ is not more than 50 amino acids.

In a particular embodiment of the above method, n is 1.

Also envisaged is the above method wherein the carrier polypeptide of the chimeric polypeptide is selected from the group consisting of:
a) a polypeptide having an amino acid sequence of SEQ ID NO: 3,
b) a polypeptide that has at least 60% amino acid identity with SEQ ID NO: 3,
c) a fragment of a polypeptide having an amino acid sequence of SEQ ID NO: 3 or a fragment of a polypeptide that has at least 60% amino acid identity with SEQ ID NO: 3,
d) a polypeptide having an amino acid sequence of SEQ ID NOS: 4-8, and
e) a polypeptide that has at least 60% amino acid identity with SEQ ID NOS: 4-8.

In the above method, the chimeric polypeptide may further comprise a signal peptide.

In one embodiment of the above method, the passenger polypeptide comprised in the chimeric polypeptide is an enzyme or a binding domain. Particularly, the passenger polypeptide comprised in the chimeric polypeptide is between 100 amino acids and 250 amino acids.

Another aspect of the application encompasses a functionalized fiber obtained by any of the above methods.

A further aspect relates to a recombinant nucleic acid molecule comprising a nucleic acid sequence encoding a chimeric polypeptide, the chimeric polypeptide comprising:
a) a carrier polypeptide comprising an amino acid sequence V/I/L-X-Q-X-G-X-X-N/Q-X-A/V/I/L-X-X-X-Q (SEQ ID NO: 32), wherein X means any amino acid,
b) a passenger polypeptide of at least 50 amino acids, and
c) optionally, a linker that couples a) to b).

More particularly, the carrier polypeptide of the chimeric polypeptide has the following structure:

(Y₂ᵢ₋₁-Xᵢ-Y₂ᵢ)ₙ, wherein
a) n is an integer from 1 to 20 and i increases from 1 to n with each repeat;
b) each Xᵢ corresponds to the amino acid sequence V/I/L-X-Q-X-G-X-X-N/Q-X-A/V/I/L-X-X-X-Q (SEQ ID NO: 32), wherein X means any amino acid; and
c) each Y₂ᵢ₋₁ and Y₂ᵢ are independently selected from 0 to 20 contiguous amino acids, wherein the total length of each Y₂ᵢ₋₁-Xᵢ-Y₂ᵢ is not more than 50 amino acids.

In one embodiment of the above recombinant nucleic acid molecule, n is 1.

In another embodiment of the recombinant nucleic acid molecule, the carrier polypeptide of the chimeric polypeptide is selected from the group consisting of:
a) a polypeptide having an amino acid sequence of SEQ ID NO: 3,
b) a polypeptide that has at least 60% amino acid identity with SEQ ID NO: 3,
c) a fragment of a polypeptide having an amino acid sequence of SEQ ID NO: 3 or a fragment of a polypeptide that has at least 60% amino acid identity with SEQ ID NO: 3,
d) a polypeptide having an amino acid sequence of SEQ ID NOS: 4-8, and
e) a polypeptide that has at least 60% amino acid identity with SEQ ID NOS: 4-8.

In another embodiment of the above recombinant nucleic acid molecule, the chimeric polypeptide further comprises a signal peptide.

In another embodiment of the above recombinant nucleic acid molecule, the passenger polypeptide comprised in the chimeric polypeptide is an enzyme or a binding domain.

Also envisaged in this application is a vector comprising any of the above recombinant nucleic acid molecules, as well as a host cell comprising any of the above recombinant nucleic acid molecules or vectors. Preferably, the host cell is a bacterial host cell, in particular a Gram-negative bacterial host cell or a Gram-positive bacterial host cell.

In one embodiment, the above host cell is genetically engineered to express, either endogenously or exogenously, a nucleic acid sequence encoding CsgG, and at least one nucleic acid sequence encoding one or more of CsgB, CsgC, CsgE, CsgF, or variants or fragments of any thereof.

In another embodiment of the above host cells, the recombinant nucleic acid molecule encoding any of the above-described chimeric polypeptides and nucleic acid sequences are expressed simultaneously.

Also, the host cell may be a component of a bacterial biofilm.

Another aspect of the application relates to a chimeric polypeptide encoded by any of the above-described recombinant nucleic acid molecules.

Also envisaged is a composition comprising one or more chimeric polypeptides encoded by one or more of the above-described recombinant nucleic acid molecules, whereby the passenger polypeptide of each chimeric polypeptide in the composition is a functionally active polypeptide. Preferably, the composition is a fiber composition. The composition may be attached to a surface, in particular, a cell surface or an artificial surface.

In yet another aspect, the application also encompasses the use of the above compositions for detecting and/or capturing of a substance, such as a protein, an organic or inorganic compound, a heavy metal, or a pollutant, in particular, the use of the composition for the chemical and/or enzymatic conversion of a substance, such as a protein, an organic or inorganic compound, a heavy metal, or a pollutant.

The application also relates to a method for producing a chimeric polypeptide in the extracellular medium of a host cell culture, the method comprising the steps of:
a) providing a host cell that is genetically engineered to express a CsgG protein, or variant or fragment thereof, and a chimeric polypeptide comprising:
   i. a carrier polypeptide comprising an amino acid sequence V/I/L-X-Q-X-G-X-X-N/Q-X-A/V/I/L-X-X-X-Q (SEQ ID NO: 32), wherein X means any amino acid,
   ii. a passenger polypeptide of 50 amino acids or more, and
   iii. optionally, a linker that couples a) to b), and
b) culturing the host cell of a) under suitable conditions to express and secrete the chimeric polypeptide into the extracellular medium,
   whereby the CsgG protein, or variant or fragment thereof, and the chimeric polypeptide are expressed concomitantly, and whereby the passenger polypeptide of the chimeric polypeptide is maintained as an active polypeptide after secretion.

In the above method, the host cell may be genetically engineered to simultaneously express CsgE, or a variant or a fragment thereof.

In one embodiment of the above method, the method comprises the step of isolating the chimeric polypeptide from the culture medium.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. ERD10 fused to CsgA is expressed on the surface of the E. coli bacteria. (Panel A) Representation of pNA1 and pNA36 vectors. pNA1 harbors 6×His-tagged (H₆) csgA under the control of the arabinose inducible P_{BAD} promoter. pNA36 is derived from pNA1 by introducing ERD10 and a flexible linker with sequence SGSGSG (L) in the SmaI site in between csgA and H₆. (Panels B, C and D) Immunofluorescence microscopy using a primary mouse anti-6×His and a secondary anti-mouse ALEXA FLUOR® 488-labeled antibody of induced DH5α (pNA36) cells (Panel B), DH5α (pBAD33) (Panel C) or DH5α (pNA48), producing ERD10-6×His in the periplasm (Panel D). (Panel E) Dot blot analysis on whole cells using a primary mouse anti-6×His antibody. LSR10 (i.e., MC4100ΔcsgA) or NVG1 (i.e., LSR10ΔcsgG) were tested, expressing either the empty vector (pBAD33), periplasmic ERD10-6×His (pNA48) or the csgA-ERD10-6×His fusion (pNA36). Cells were left untreated (−) or treated with lysozyme and EDTA (+) prior to blotting. (Panel F) Anti-6×His immunogold TEM of LSR10 (pNA36), scale bar is 100 nm. (Panel G) TEM micrographs of the negative control LSR10 (pBAD33), scale bar represents 200 nm.

FIG. 2. Expression of different CsgA fusion proteins on the surface of bacteria. Immunofluorescence microscopy, using a primary mouse anti-6×His and a secondary anti-mouse ALEXA FLUOR® 488-labeled antibody of E. coli LSR10 expressing the different CsgA fusion proteins. LSR10 (pBAD33), harboring the empty vector (pBAD33 in figure), LSR10 (pNA15) (A-Nb208), LSR10 (pNA32) (A-FedF), LSR10 (pNA30) (A-FimC), LSR10 (pNA34) (A-mCherry), LSR10 (pNA29) (A-RNase1), LSR10 (pNA31) (A-Bla), and LSR10 (pNA33) (A-PhoA).

FIG. 3. Display of heterologous proteins fused to CsgA. (Panel A) Whole cell ELISA of MC4100 (CsgA) or E. coli LSR10 producing the different CsgA fusion proteins. Anti-6×His (His) and anti-peptidoglycan (pep) were used as primary antibodies: results are normalized to anti-E. coli antibodies and shown in arbitrary units (A.U.). SD are shown for three independent experiments, done in triplicate. Statistics were done with the Mann-Whitney test, using pBAD33 as reference (for anti-pep response: *p<0.05, **p<0.001). (Panels B and C) Protease surface accessibility of proteins fused to CsgA. LSR10 cells harboring different proteins fused to CsgA were treated with formic acid and cell lysates were subjected to SDS-PAGE and subsequent Western blotting using an anti-6×His mAb (Panel B) or an anti-DsbA antiserum (Panel C). Prior to formic acid treatment, cells were incubated with proteinase K (Prot K) (+), or PBS buffer (−). As a control, LSR10 (pNA15) cells were subjected to sonication prior to Prot K treatment (A-Nb208 sonic). § indicates the bands corresponding to the respective fusion proteins, and ° the band corresponding to the passenger proteins only.

FIG. 4. Nb208 fused to CsgA is expressed and active on the surface of E. coli bacteria. (Panel A) Immunofluorescence microscopy, using a primary mouse anti-histidine and a secondary anti-mouse ALEXA FLUOR® 488-labeled antibody, of induced DH5α (pNA15) cells (Panels B, C and D). Fluorescence microscopy of binding of exogenously added green fluorescent protein (GFP) to induced LSR10 (pNA15) cells (Panel B) or LSR10 (pCA747) (pNA18) cells expressing Nb208 in the periplasm and nanobody cAbLys3 fused to CsgA, after 48 hours (Panel C) or 72 hours of induction (Panel D).

FIG. 5. CsgG-mediated secretion is compatible with small folded CsgA-fused passengers. (Panels A-D) Disulfide formation in Nb208 is necessary for GFP binding. (Panels A and C) Anti-6×His and anti-mouse ALEXA FLUOR® 594 IF of induced LSR10 (pNA35) expressing CsgA-Nb208^{C22S} (Panel A) or MC1000 ΔdsbA (pNA15) expressing CsgA-Nb208 (Panel C). (Panels B and D) Exogenously added GFP fails to bind to induced LSR10 (pNA35) (Panel B) or MC1000 ΔdsbA (pNA15) (Panel D). (Panels E-H) The conformationally selective anti-FedF nanobody Nb231 recognizes folded FedF on the surface of bacteria. (Panel E insert) Dot blot of boiled (B) and native FedF (NB), using Nb231. (Panels E and F) IF using a FITC-labeled Nb231 of induced LSR10 (pNA32), expressing the CsgA-FedF fusion protein and untreated (Panel E) or treated (Panel F) with DTT and 2-ME prior to IF. (Panels G and H) IF of induced MC1000 ΔdsbA (pNA32), stained with an anti-6×His mAb and an anti-mouse ALEXA FLUOR® 594-labeled secondary antibody (Panel G) or with the FITC-labeled Nb231 (Panel H).

FIG. 6. TEM analysis of secreted CsgA-Nb208 deposits. (Panel A) Negative TEM image of LSR10 (pNA15) shows the predominant formation of a dense matrix of positively staining aggregates. (Panel B) MC4100 showing native curli fibers as revealed by negative staining TEM. (Panel C) Besides aggregates, TEM and Ni-NTA-gold (5 nm) staining shows LSR10 (pNA15) displays negatively staining filamentous threads that contain CsgA-Nb208-6×His. (Panel D) Ni-NTA-gold-labeled CsgA-6×His fibrils as found on the surface of LSR10 (pNA1). Black bars indicate a 100 nm scale.

FIG. 7. Western blotting and TEM analysis of secreted CsgA-fusions and SDS-insoluble surface-bound filaments. (Panel A) Anti-His Western blot analysis of cell lysates of LSR10 cells expressing CsgA-Nb208 (pNA15), CsgA-FedF (pNA32), CsgA-RNase1 (pNA29) or CsgA-ERD10 (pNA36), treated with (FA+) or without (FA−) formic acid. (Panels B, C, and D) SDS-insoluble material was isolated from LSR10 cells expressing different fusion proteins, visualized by negative staining TEM in case of the CsgA-Nb208 fusion (Panel B), or after formic acid treatment subjected to SDS-PAGE, followed by anti-6×His (Panel C) or anti-CsgA (Panel D) Western blotting. Arrow, ° and § indicate the band corresponding to SDS-insoluble CsgA-fusions, the fused proteins and the various intact fusion proteins, respectively. Black bar indicates a 100 nm scale.

FIG. 8. Structures of the different passenger proteins fused to CsgA, with their respective size, number of disulfide bonds and transverse diameter. ERD10 is an intrinsically disordered protein (IDP), so no transverse diameter is calculated.

FIGS. 9A-9C. Detection of disulphide bridges in RNase1 by mass spectrometry. (FIG. 9A) ESI-Q-TOF spectra of tryptic peptides from periplasmic RNase1 (upper panel) and CsgA-RNase1 (lower panel). (FIG. 9B) Location of the four canonical disulphide pairs in RNase1 (SEQ ID NO: 18). The tryptic peptides detected by peptide mass fingerprint in CsgA-RNase1 spectrum are highlighted in bold blue in the protein sequence. (FIG. 9C) Based on their charge and m/z ratio, tryptic peptides bound by a disulphide bond were detected only in periplasmic RNase1 spectrum. The isotopic peak distributions of these peptide pairs are represented (the color code for the four disulphide bridges is the same as in FIG. 9B). These peaks were not clearly observed in the mass spectrum of CsgA-RNase1 tryptic peptides. The identities of the disulphide bound peptides detected in periplasmic RNase1 were confirmed by microsequencing by tandem mass spectrometry.

FIG. 10. E. coli LSR10 bacteria harboring a CsgA-NB208 fusion lacking N22 still express NB208 on their surface. (Panel A) Transmission electron microscopy (TEM) of LSR10 (pNA26), scale bar represents 1 μm. (Panel B) Fluorescence microscopy of binding of the green fluorescent protein (GFP) to induced LSR10 (pNA26) cells.

FIG. 11. E. coli LSR10 bacteria harboring a CsgA-NB208 fusion lacking R2 to R5 still express NB208 on their surface. (Panel A) Transmission electron microscopy (TEM) of LSR10 (pNA21), scale bar represents 1 μm. (Panel B) Fluorescence microscopy of binding of the green fluorescent protein (GFP) to induced LSR10 (pNA21) cells.

FIG. 12. E. coli LSR10 bacteria harboring a CsgA-NB208 fusion lacking R1 still express NB208 on their surface. (Panel A) Transmission electron microscopy (TEM) of LSR10 (pNA25), scale bar represents 200 nm. (Panel B) Fluorescence microscopy of binding of the green fluorescent protein (GFP) to induced LSR10 (pNA25) cells.

FIG. 13. Congo red binding of E. coli LSR10 bacteria harboring a CsgA-NB208 fusion lacking different CsgA repeats. pBAD33 is the empty vector control.

FIG. 14. Congo red binding of E. coli LSR10 cells producing the different CsgA repeats fused to NB208. PC stands for positive control, i.e., LSR10 (pNA15). NC is the negative LSR10 (pBAD33) control. R1 to R5 represent LSR10 containing pSB1, pSB2, pSB3, pSB4 or pSB5, respectively.

FIG. 15. E. coli LSR10 bacteria harboring a R2-NB208 fusion still express NB208 on their surface. (Panel A) Transmission electron microscopy (TEM) of LSR10 (pSB2), scale bar represents 500 nm. (Panel B) Fluorescence microscopy of binding of the green fluorescent protein (GFP) to induced LSR10 (pSB2) cells.

FIG. 16. TEM analysis of secreted CsgA-Nb208 deposits. Ni-NTA-gold (5 nm) staining shows MC4100 (pNA15) displays negatively staining filamentous threads that contain CsgA-Nb208-6×His. Scale bar indicates a 100 nm scale.

FIG. 17. Broadening the host range of curli display to Salmonella. Fluorescence microscopy of binding of exogenously added green fluorescent protein (GFP) to induced Salmonella χ3000 (pNA15) cells.

FIG. 18. Secretion and fiber formation of CsgA-fusion proteins by Gram-positive bacteria. Transmission electron microscopy (TEM) of Lactococcus lactis negative control (Panel A; scale bar represents 1 μm), L. lactis (pEXP424) harboring the CsgA-NB208 fusion protein (Panel B; scale bar represents 500 nm) or L. lactis (pEXP437) harboring the CsgA-Bla fusion protein (Panel C; scale bar represents 100 nm).

FIG. 19. In vitro grown CsgA fibers display the NB208 fusion protein in its active conformation. Ni-NTA gold (5 nm) binding to CsgA-NB208-His fibers grown in vitro shows the intact fusion is present in the fibers (Panel A). GFP coupled to nanogold binds specifically to the CsgA-NB208-His fibers, indicating NB208 is functionally folded (Panel B). Scale bars represent 100 nm.

FIG. 20. In vitro grown CsgA fibers coupled to a solid surface. Coupling of in vitro CsgA-6×His fibers to carboxylate-modified magnetic microparticles. Transmission electron microscopy (TEM) (Panel A; scale bar represents 500 nm) and anti-histidine immunofluorescence microscopy of CsgA-6×His fibers grown on magnetic particles (Panel B).

### DETAILED DESCRIPTION

This disclosure will be described with respect to particular embodiments and with reference to certain drawings but the disclosure is not limited thereto but only by the claims. Any reference signs in the claims shall not be construed as limiting the scope. The drawings described are only schematic and are non-limiting. In the drawings, the size of some of the elements may be exaggerated and not drawn to scale for illustrative purposes. Where the term “comprising” is used in the present description and claims, it does not exclude other elements or steps. Where an indefinite or definite article is used when referring to a singular noun, e.g., “a,” “an,” or “the,” this includes a plural of that noun unless something else is specifically stated. Furthermore, the terms “first,” “second,” “third,” and the like, in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

Unless otherwise defined herein, scientific and technical terms and phrases used in connection with this disclosure shall have the meanings that are commonly understood by those of ordinary skill in the art. Generally, nomenclatures used in connection with, and techniques of molecular and cellular biology, genetics and protein and nucleic acid chemistry and hybridization described herein are those well-known and commonly used in the art. The methods and techniques of this disclosure are generally performed according to conventional methods well known in the art and as described in various general and more specific references that are cited and discussed throughout the present specification unless otherwise indicated. See, for example, Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002).

### DEFINITIONS

As used herein, the terms “polypeptide,” “protein,” and “peptide” are used interchangeably herein, and refer to a polymeric form of amino acids of any length, which can include coded and non-coded amino acids, chemically or biochemically modified or derivatized amino acids, and polypeptides having modified peptide backbones. Throughout the application, the standard one letter notation of amino acids will be used. Typically, the term “amino acid” will refer to “proteinogenic amino acid,” i.e., those amino acids that are naturally present in proteins. Most particularly, the amino acids are in the L isomeric form, but D amino acids are also envisaged.

As used herein, the terms “nucleic acid molecule,” “polynucleotide,” “polynucleic acid,” and “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

Any of the peptides, polypeptides, nucleic acids, etc., disclosed herein may be “isolated” or “purified.” “Isolated” is used herein to indicate that the material referred to is (i) separated from one or more substances with which it exists in nature (e.g., is separated from at least some cellular material, separated from other polypeptides, separated from its natural sequence context), and/or (ii) is produced by a process that involves the hand of man such as recombinant DNA technology, chemical synthesis, etc.; and/or (iii) has a sequence, structure, or chemical composition not found in nature. “Purified” as used herein denote that the indicated nucleic acid or polypeptide is present in the substantial absence of other biological macromolecules, e.g., polynucleotides, proteins, and the like. In one embodiment, the polynucleotide or polypeptide is purified such that it constitutes at least 90% by weight, e.g., at least 95% by weight, e.g., at least 99% by weight, of the polynucleotide(s) or polypeptide(s) present (but water, buffers, ions, and other small molecules, especially molecules having a molecular weight of less than 1000 Daltons, can be present).

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Determining the percentage of sequence identity can be done manually, or by making use of computer programs that are available in the art. Examples of useful algorithms are PILEUP (Higgins & Sharp, CABIOS 5:151 (1989), BLAST and BLAST 2.0 (Altschul et al., J. Mol. Biol. 215:403 (1990)) and ClustalW and ClustalW2 (Larkin et al., Bioinformatics 23:2947 (2007)) or Multalin (F. Corpet, Nucl. Acids Res. 16:10881 (1988)). Software for performing BLAST analyses is publicly available through the National Center for Biotechnology Information (World Wide Web at ncbi.nlm.nih.gov/), multiple sequence alignments using ClustalW or ClustalW2 can be performed through the public tools provided by the European Bioinformatics Institute (World Wide Web at ebi.ac.uk/Tools).

“Similarity” refers to the percentage number of amino acids that are identical or constitute conservative substitutions. Similarity may be determined using sequence comparison programs such as GAP (Deveraux et al. 1984, Nucleic Acids Research 12:387-395) or FASTA (World Wide Web at fasta.bioch.virginia.edu/fasta_www2/fasta_list2.shtml; Pearson and Lipman, Proc. Natl. Acad. Sci. U.S.A. 85:2444 (1988)). In this way, sequences of a similar or substantially different length to those cited herein might be compared by insertion of gaps into the alignment, such gaps being determined, for example, by the comparison algorithm used by GAP.

As used herein, “conservative substitution” is the substitution of amino acids with other amino acids whose side chains have similar biochemical properties (e.g., are aliphatic, are aromatic, are positively charged, . . . ) and is well known to the skilled person. Non-conservative substitution is then the substitution of amino acids with other amino acids whose side chains do not have similar biochemical properties (e.g., replacement of a hydrophobic with a polar residue). Conservative substitutions will typically yield sequences that are not identical anymore, but still highly similar. As used herein, the term “hydrophobic amino acids” refers to the following 13 amino acids: isoleucine (I), leucine (L), valine (V), phenylalanine (F), tyrosine (Y), tryptophan (W), histidine (H), methionine (M), threonine (T), lysine (K), alanine (A), cysteine (C), and glycine (G). The term “aliphatic amino acids” refers to I, L or V residues. The term “charged amino acids” refers to arginine (R), lysine (K)—both positively charged; and aspartic acid (D), glutamic acid (E)—both negatively charged. The term “aromatic amino acids” refers to phenylalanine (F), tryptophan (W), tyrosine (Y), and histidine (H).

The term “recombinant” or “heterologous” when used in reference to a cell, nucleic acid, protein or vector, indicates that the cell, nucleic acid, protein or vector has been modified by the introduction of a non-native nucleic acid or protein or the alteration of a native nucleic acid or protein, or that the cell is derived from a cell so modified. Thus, for example, recombinant cells express nucleic acids or polypeptides that are not found within the native (non-recombinant) form of the cell or express native genes that are otherwise abnormally expressed, under expressed, over expressed or not expressed at all. The non-native nucleic acids or polypeptides are referred to as being heterologous, e.g., of a non-native origin.

As used herein, the term “carrier polypeptide” or “carrier protein” refers to a polypeptide that is secreted by an appropriate secretion system of a host cell and that has the capability and characteristics to, but does not need to, polymerize into a fiber structure. Within the context of this disclosure, the carrier polypeptide is derived from a naturally occurring bacterial protein, in particular, a fiber subunit protein, which is all defined in more detail further herein. It will be appreciated that a carrier polypeptide as used herein can be identical to a naturally occurring bacterial protein or can be a variant or a fragment derived thereof (as defined further herein), as long as it retains the capability to polymerize in vivo or in vitro. A fiber may comprise identical or different fiber subunits. In nature, a fiber is typically composed of a major and minor fiber subunit, reflecting either a high or low copy number in the fiber, respectively.

As used herein, the term “passenger polypeptide” or “passenger protein” is defined as a polypeptide that, when fused to a carrier polypeptide, is co-secreted and, if applicable, co-polymerized into a fiber structure.

The terms “chimeric polypeptide,” “chimeric protein,” “fusion polypeptide,” and “fusion protein” are used interchangeably herein and refer to a protein that comprises at least two separate and distinct regions that may or may not originate from the same protein. For example, a signal peptide linked to a protein of interest, wherein the signal peptide is not normally associated with the protein of interest, would be termed a “chimeric polypeptide” or “chimeric protein.” Or, two proteins or two protein domains that are not normally associated with each other, are other examples of chimeric polypeptides. A convenient means for linking or fusing two polypeptides is by expressing them as a fusion protein from a recombinant nucleic acid molecule, which comprises, for example, and within the present scope, a first polynucleotide encoding a carrier polypeptide operably linked to a second polynucleotide encoding a passenger polypeptide. Otherwise, the polypeptides comprised in a fusion protein can be linked through peptide bonds or may even be chemically linked. Typically, such a chimeric polypeptide will not exist as a contiguous polypeptide in a protein encoded by a gene in a non-recombinant genome. The term “chimeric polypeptide” and equivalents thus refers to a non-naturally occurring molecule, which means that it is manmade.

As used herein, the term “expression” refers to the process by which a polypeptide is produced based on the nucleic acid sequence of a gene. The process includes both transcription and translation.

The term “operably linked” as used herein refers to a linkage in which a regulatory sequence is contiguous with the gene of interest to control the gene of interest, as well as regulatory sequences that act in trans or at a distance to control the gene of interest. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art. In a “fusion protein” or “chimeric polypeptide,” within the scope of this disclosure, a DNA sequence for a carrier polypeptide is operably linked to a DNA sequence of a passenger polypeptide when both are transcribed to a continuous messenger RNA and when both coding sequences are translated into a continuous polypeptide.

The term “regulatory sequence” as used herein refers to polynucleotide sequences that are necessary to affect the expression of coding sequences to which they are operably linked. Expression control sequences are sequences that control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and, when desired, sequences that enhance protein secretion. The nature of such control sequences differs depending upon the host organism. The term “control sequences” is intended to include, at a minimum, all components whose presence is essential for expression, and can also include additional components whose presence is advantageous, for example, leader sequences and fusion partner sequences.

The term “conformation” or “conformational state” of a protein refers generally to the range of tridimensional structures that a polypeptide may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three-dimensional folding of a polypeptide chain), and quaternary structure (e.g., interactions of a polypeptide chain with other protein subunits). Post-translational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, FTIR, circular dichroism, NMR, or spin labeling, among other methods. For a general discussion of protein conformation and conformational states, one is referred to Cantor and Schimmel, Biophysical Chemistry, Part I: The Conformation of Biological Macromolecules, W.H. Freeman and Company, 1980, and Creighton, Proteins: Structures and Molecular Properties, W.H. Freeman and Company, 1993.

As used herein, the phrase “polypeptide in a functional conformation” or “functional polypeptide” or a “functionally active polypeptide” refers to a polypeptide that has adopted a particular functional conformational state, including a native conformation. As used herein, a “functional conformation” or a “functional conformational state” refers to the fact that a protein or polypeptide possesses a particular structural conformation that determines a particular protein activity (e.g., antigen binding activity, ligand binding activity, chemical activity, enzymatic activity, etc.). It should thus be clear that “a functional conformation” is meant to cover any conformation, having any activity, and is not meant to cover the denatured states of proteins. As used herein, the phrase “polypeptide in its native conformation” refers to the functional conformation of the polypeptide as adopted under its native conditions, e.g., as found under physiological conditions in its natural host and localization. It should be noted that the “native conformation” of a polypeptide is not per se restricted to a single conformation, but can encompass a dynamic range of conformations or a number of discrete conformations. The term “polypeptide in a functional conformation” is not meant to include linear epitopes or linear peptides.

As used herein, the term “transverse diameter” is defined as the diameter measured perpendicular to the longitudinal axis of an object, e.g., a protein in its tertiary or quaternary state. As used herein, an object's maximum transverse diameter can be understood to be equal to the minimal inner diameter of a hollow cylinder that allows inclusion or passage of the object.

As used herein, the terms “determining,” “measuring,” “assessing,” “monitoring,” and “assaying” are used interchangeably and include both quantitative and qualitative determinations.

The term “signal peptide” as used herein is defined as a short peptide of between 5 and 40 amino acids long, that when located at the N-terminus, directs the newly synthesized polypeptide toward the general secretory pathway or the Twin Arginine Transport (TAT) pathway. Synonyms include “signal sequence,” “leader sequence,” and “leader peptide”; these terms are used interchangeably herein. The signal peptide can or cannot be removed from the translocated polypeptide by post-translational, proteolytic processing. Examples are provided further in the specification.

The term “biofilm,” as used herein, is an aggregate of microorganisms in which cells adhere to each other and/or to a surface. These adherent cells are frequently embedded within a self-produced matrix generally composed of extracellular DNA, proteins, and polysaccharides in various configurations. Biofilms can contain many different types of microorganism, e.g., bacteria, archaea, protozoa, fungi and algae. However, monospecies biofilms occur as well. Microorganisms living in a biofilm usually have significantly different properties from free-floating (planktonic) microorganisms of the same species, as a result of the dense and protected environment of the film. For example, increased resistance to detergents and antibiotics is often observed, as the dense extracellular matrix and the outer layer of cells protect the interior of the community.

The term “vector” as used herein is intended to refer to a nucleic acid molecule capable of transporting another nucleic acid molecule to which it has been linked. The vector may be of any suitable type including, but not limited to, a phage, virus, plasmid, phagemid, cosmid, bacmid or even an artificial chromosome. Certain vectors are capable of autonomous replication in a host cell into which they are introduced (e.g., vectors having an origin of replication that functions in the host cell). Other vectors can be integrated into the genome of a host cell upon introduction into the host cell, and are thereby replicated along with the host genome. Moreover, certain preferred vectors are capable of directing the expression of certain genes of interest. Such vectors are referred to herein as “recombinant expression vectors” (or simply, “expression vectors”). Suitable vectors have regulatory sequences, such as promoters, enhancers, terminator sequences, and the like as desired and according to a particular host organism (e.g., bacterial cell, yeast cell). Typically, a recombinant vector according to this disclosure comprises at least one “chimeric gene” or “expression cassette.” Expression cassettes are generally DNA constructs preferably including (5′ to 3′ in the direction of transcription): a promoter region, a polynucleotide sequence, homologue, variant or fragment thereof of this disclosure, operably linked with the transcription initiation region, and a termination sequence including a stop signal for RNA polymerase and a polyadenylation signal. It is understood that all of these regions should be capable of operating in biological cells, in particular, bacterial cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The term “host cell,” as used herein, is intended to refer to a cell into which a recombinant vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein. A host cell may be an isolated cell or cell line grown in culture or may be a cell that resides in a living tissue or organism. In particular, host cells are of bacterial or fungal origin, but may also be of plant or mammalian origin. The wordings “host cell,” “recombinant host cell,” “expression host cell,” “expression host system,” and “expression system,” are intended to have the same meaning and are used interchangeably herein.

This disclosure provides tools and methods for the recombinant production, transport and secretion of chimeric polypeptides by bacterial host cells. The chimeric polypeptides as described herein comprise a carrier polypeptide moiety characterized by its ability to self-polymerize into a fiber and a passenger polypeptide moiety that is carried along with the carrier polypeptide moiety. The chimeric polypeptides thus essentially comprise a passenger polypeptide that is fused to a carrier polypeptide. The carrier polypeptides are designed polypeptides that hold properties and sequence characteristics from the curlin repeat family of proteins. The chimeric polypeptides are produced by a bacterial cell, either a Gram-positive or a Gram-negative bacterial cell. When produced by a Gram-negative (diderm) bacterial cell, they can be isolated from the bacterial cell or secreted to the extracellular environment by virtue of the secretion machinery responsible for the assembly of curli-like fibers (also called Type VIII secretion system or nucleation-precipitation pathway), which minimally encompasses a CsgG-like lipoprotein and can include the accessory proteins CsgE or CsgF. Upon secretion, the chimeric polypeptides may self-assemble into curli-like fibers by virtue of the polymerizing nature of the carrier polypeptide. The tools and methods as described herein additionally provide for the functional display of polypeptides along filamentous fibers on the producing host cell surface or on foreign surfaces.

Thus, one aspect of this disclosure relates to a recombinant nucleic acid molecule comprising a nucleic acid sequence encoding a chimeric polypeptide which is a fusion protein of different moieties, in particular, comprising at least a carrier polypeptide moiety and a passenger polypeptide moiety.

In particular, the disclosure provides for a recombinant nucleic acid molecule comprising a nucleic acid sequence encoding a chimeric polypeptide, the chimeric polypeptide comprising:
a) a carrier polypeptide comprising an amino acid sequence V/I/L-X-Q-X-G-X-X-N/Q-X-A/V/IL-X-X-X-Q (SEQ ID NO: 32) wherein X means any amino acid,
b) a passenger polypeptide of at least 50 amino acids, and
c) optionally, a linker that couples a) to b).

### Carrier Polypeptides

In general, several naturally occurring bacterial surface proteins can be used to present proteins on the bacterial surface, including S-layer proteins, lipoproteins, autotransporters and subunits of surface appendages. Structural subunits of fibrillar structures such as flagella and pili are particularly useful to transport proteins onto the cell surface because of their natural function and/or their highly organized multi-subunit features. The terms pili (hair-like structures) and fimbriae (threads), collectively referred to as “pili,” are generally being used to indicate exterior appendages formed by any of the following biosynthetic pathways: the chaperone-usher and alternate chaperone-usher pathways, Type II-like secretion systems (Type IV pili), Type III secretion systems, Type IV secretion systems, Type VIII secretion system (also called extracellular nucleation-precipitation), or by sortase-mediated assembly pathways. Pili are involved in numerous essential biological processes such as, for example, recognition and colonization of target surfaces, biofilm formation, shielding and host subversion, motility, protein and nucleic acid secretion and/or uptake, and signaling events. “Flagella” represent the other main type of filamentous multi-subunit surface organelles on bacteria. They are considered unique motility organelles not only used for swimming but also essential for swarming. Visualized by electron microscopy (EM), flagella are thicker, longer, and less numerous than pili. Invariably, these two types of surface appendages are built up of one or a few repeating (glyco)protein subunits that are covalently or noncovalently attached to linear or branched structures. The various classes of bacterial surface appendages along with their biosynthetic pathways and structural properties are reviewed by Van Gerven et al. (2011), the content of which is incorporated herein by reference.

Within the scope of the disclosure, a preferred class of bacterial fiber subunits for the design of carrier proteins for functional display of proteins is the class of fiber subunit components of “curli fibers” or “curli.” As used herein, the term “curli” refers to unbranched, highly aggregative flexible filaments of 4-7 nm diameter and are the major proteinaceous component of the extracellular matrix produced by many bacteria, e.g., many Enterobacteriaceae such as E. coli and Salmonella spp. (Barnhart et al. 2006). In Salmonella typhymurium, these are called thin aggregative fimbriae (Tafi) (Collinson et al. 1991). Curli are formed by means of the extracellular nucleation-precipitation (ENP) pathway, also referred to as Type VIII secretion system (T8SS). Native curli fibers exhibit structural and biochemical properties of amyloids, e.g., they are non-branching, cross-beta sheet rich fibers (e.g., showing characteristic fiber diffraction signals at 4.7 Å and 10 Å) that are resistant to protease digestion and denaturation by 10% SDS, and bind to amyloid-specific moieties such as thioflavin T, which fluoresces when bound to amyloid, and Congo red, which produces a unique spectral pattern (“red shift”) in the presence of amyloid. Native curli fibers require formic acid treatment for depolymerization, unlike amorphous or colloidal protein aggregates or other filamentous organelles such as pili and flagella. Curli fibers are involved in adhesion to surfaces, cell aggregation, and biofilm formation. Curli also mediate host cell adhesion and invasion, and they are potent inducers of the host inflammatory response. It will be appreciated that the term “curli” also includes native-like curli fibers whereby the filamentous threads can have a different fibrillous structure but that retain the characteristic to be resistant to denaturation by 10% SDS.

In nature, curli subunits are secreted as monomeric subunits that polymerize on the extracellular surface upon contact with growing fibers or a surface-exposed nucleator protein (Chapman et al. 2002). Taking the curli biogenesis pathway in Escherichia coli as a non-limiting example, curli are assembled by a process in which the major fiber subunit polypeptide, CsgA (SEQ ID NO: 1), is nucleated into a fiber by the minor fiber subunit polypeptide, CsgB (SEQ ID NO: 24), or by pre-existing CsgA polymers. CsgA and CsgB are about 30% identical at the amino acid level and contain an imperfect five-fold internal repeat symmetry characterized by conserved polar residues. The assembly process is believed to involve addition of soluble polypeptides to the growing fiber tip. Thus, both subunits are incorporated into the fiber, although CsgA is the major protein constituent. In living bacteria, curli formation likely involves activities of several additional polypeptides encoded by other Csg genes (CsgD (SEQ ID NO: 25), CsgE (SEQ ID NO: 26), CsgF (SEQ ID NO: 27), CsgG (SEQ ID NO: 28)), whereas these polypeptides are not required for curli formation in vitro. CsgG forms a pore in the outer membrane and is important for the stability and secretion of CsgA, CsgB and CsgF. The latter plays a role in the stability and nucleation activity of CsgB. Other curli proteins are CsgD, the transcriptional activator for the csgBAC-operon, CsgE, which potentially has chaperone properties and CsgC, which possibly has oxido-reductase activity and may possibly bind CsgG.

“CsgA polypeptide” or simply “CsgA,” as used herein, encompasses any polypeptide having an amino acid sequence of a naturally occurring bacterial CsgA polypeptide as well as variants of a polypeptide having an amino acid sequence of a naturally occurring bacterial CsgA polypeptide. A CsgA polypeptide variant is at least 40%, 50%, 60%, 70%, 80%, 85%, 90%, 95%, 96%, 97%, 98%, 99% identical to or similar (as defined herein) to a naturally occurring CsgA polypeptide. Naturally occurring CsgA polypeptides are known in the art and amino acid sequences of CsgA polypeptides from a large number of bacteria have been identified. One of skill in the art will readily be able to find CsgA sequences by searching databases such as GenBank, which are publicly available through the National Center for Biotechnology Information (NCBI; see the World Wide Web at ncbi.nlm.nih.gov). CsgA polypeptides characteristically encompass multiple copies of a 20-30 amino acid repeat known as curlin repeat (PFAM domain PF07012: World Wide Web at pfam.sanger.ac.uk/family/PF07012), herein incorporated by reference. In general, CsgA polypeptides have an N-terminal secretion signal for transport through the SEC-system, which is cleaved off, followed by multiple copies of imperfect repeats containing an S-(X)₅-Q-(X)₄-N-(X)₅-Q motif (SXXXXXQXXXXNXXXXXQ, SEQ ID NO: 29, wherein X means any amino acid) and providing the amyloidogenic core of the protein (Collison et al. 1999; Wang and Chapman 2008). As an illustration, E. coli CsgA (SEQ ID NO: 1) consists of an N-terminal secretion signal (MKLLKVAAIAAIVFSGSALA; SEQ ID NO: 30) that is cleaved off, an N-terminal domain of 22 amino acids (GVVPQYGGGGNHGGGGNNSGPN, SEQ ID NO: 31) that is believed to provide the targeting sequence for CsgG-mediated secretion, and a C-terminal amyloidogenic core (SEQ ID NO: 3), containing five strongly conserved repeats, R1-R5 (SEQ ID NO: 4 to 8). See also Table 1.

It is shown in this disclosure that carrier polypeptides derived from CsgA polypeptide subunits of bacterial curli fibers are versatile tools and allow the secretion of a fused passenger polypeptide to the extracellular environment of the producing bacterium and allows for its incorporation into fibers, where it is displayed along the length of the fiber and retains its functional conformation. These are referred to herein as “functionalized fibers.” Such functionalized fibers of the fusion protein can be formed on the cell surface of the producing bacterium, or can be nucleated onto a foreign surface that is exposed to a solution containing the fusion protein. In this disclosure, it is shown that the carrier polypeptide derived from CsgA at least comprises the following amino acid sequence: V/I/L-X-Q-X-G-X-X-N/Q-X-A/V/I/L-X-X-X-Q (SEQ ID NO: 32), wherein X means any amino acid, and minimal sequences needed for a carrier polypeptide to be secreted by a bacterial host cell and to allow subsequent polymerization into fibers are defined (as described further herein). Advantageously, the fiber composition can thus be designed according to needs and applications, by (1) adapting the sequence of the carrier polypeptide at permissive sites (e.g., where the amino acid can be freely chosen), and/or (2) varying the nature and number of the passenger polypeptides, and/or (3) designing a suitable fusion construct, and/or (4) co-production and secretion of multiple carrier-passenger fusion proteins with different passenger polypeptides in order to obtain fibers of mixed passenger composition, and/or (5) co-production and secretion of the carrier-passenger fusion polypeptide(s) with a carrier polypeptide in order to modulate the density of the passenger display in the fiber.

It will thus be understood that the carrier polypeptide moiety of this disclosure refers to a polypeptide derived from a curlin repeat polypeptide as defined hereinbefore. Here, the sequence constraints of the carrier polypeptide that forms part of the chimeric polypeptide as described herein will be explained in more detail.

According to a preferred embodiment, the carrier polypeptide of the chimeric polypeptide as described herein has the following structure: (Y₂ᵢ₋₁-Xᵢ-Y₂ᵢ)ₙ, wherein:
n is an integer from 1 to 20 and i increases from 1 to n with each repeat;
each Xᵢ corresponds to the amino acid sequence V/I/L-X-Q-X-G-X-X-N/Q-X-A/V/I/L-X-X-X-Q (SEQ ID NO: 32), wherein X means any amino acid; and
each Y₂ᵢ₋₁ and Y₂ᵢ are independently selected from 0 to 20 contiguous amino acids, wherein the total length of each Y₂ᵢ₋₁-Xᵢ-Y₂ᵢ is not more than 50 amino acids.

As mentioned, in the above formula, n is an integer from 1 to 20 and i increases from 1 to n with each repeat. In other words, i starts at 1 and is increased with 1 with each repeat until n is reached; or i is the number of the repeat (and is an integer from 1 to n).

The formula thus encompasses the following structures:

Y₁-X₁-Y₂ (i.e., n=1),

Y₁-X₁-Y₂-Y₃-X₂-Y₄ (i.e., n=2),

Y₁-X₁-Y₂-Y₃-X₂-Y₄-Y₅-X₃-Y₆ (i.e., n=3),

Y₁-X₁-Y₂-Y₃-X₂-Y₄-Y₅-X₃-Z₃-Y₇-X₄-Y₈ (i.e., n=4), and

Y₁-X₁-Y₂-Y₃-X₂-y₄-Y₅-X₃-Y₆-Y₇-X₄-Y₈-Y₉-X₅-Y₁₀ (i.e., n=5), etc. wherein each numbered X and Y are as defined above.

Non-limiting examples of suitable carrier polypeptides that have the structure (Y₂ᵢ₋₁-Xᵢ-Y₂ᵢ)ₙ as defined above include:

Y₁-Y₁-Y₂ (i.e., n = 1):
(SEQ ID NO: 4)
SELNIYQYGGGNSALALQTDARN
(SEQ ID NO: 5)
SDLTITQHGGGNGADVGQGSDD
(SEQ ID NO: 6)
SSIDLTQRGFGNSATLDQWNGKN
(SEQ ID NO: 7)
SEMTVKQFGGGNGAAVDQTASN
(SEQ ID NO: 8)
SSVNVTQVGFGNNATAHQY
Y₁-X₁-Y₂-Y₃-X₂-Y₄ (i.e., n = 2):
(SEQ ID NO: 9)
SELNIYQYGGGNSALALQTDARNSDLTITQHGGGNGADVGQGSDD
Y₁-X₁-Y₂-Y₃-X₂-Y₄-Y₅-X₃-Y₆ (i.e., n = 3):
(SEQ ID NO: 10)
SELNIYQYGGGNSALALQTDARNSDLTITQHGGGNGADVGQGSDD
SSIDLTQRGFGNSATLDQWNGKN
Y₁-X₁-Y₂-Y₃-X₂-Y₄-Y₅-X₃-Z₃-Y₇-X₄-Y₈
(i.e., n = 4):
(SEQ ID NO: 11)
SELNIYQYGGGNSALALQTDARNSDLTITQHGGGNGADVGQGSDD
SSIDLTQRGFGNSATLDQWNGKNSEMTVKQFGGGNGAAVDQTASN
(SEQ ID NO: 12)
SDLTITQHGGGNGADVGQGSDDSSIDLTQRGFGNSATLDQWNGKN
SEMTVKQFGGGNGAAVDQTASNSSVNVTQVGFGNNATAHQY
Y₁-X₁-Y₂-Y₃-X₂-Y₄-Y₅-X₃-Y₆-Y₇-X₄-Y₈-Y₉-X₅-Y₁₀
(i.e., n = 5):
(SEQ ID NO: 3)
SELNIYQYGGGNSALALQTDARNSDLTITQHGGGNGADVGQGSDD
SSIDLTQRGFGNSATLDQWNGKNSEMTVKQFGGGNGAAVDQTASN
SSVNVTQVGFGNNATAHQY

In more specific embodiments, the carrier polypeptide of the chimeric polypeptide as described herein has the following structure: (Y₂ᵢ₋₁-Xᵢ-Y₂ᵢ)ₙ, as defined above, wherein n is an integer from 1 to 15, from 1 to 10, from 1 to 9, from 1 to 8, from 1 to 7, from 1 to 6, from 1 to 5, from 1 to 4, from 1 to 3, from 1 to 2. In one particular embodiment, n is 1.

In other specific embodiments, the carrier polypeptide of the chimeric polypeptide as described herein has the following structure: (Y₂ᵢ₋₁-Xᵢ-Y₂ᵢ)ₙ, as defined above, wherein each Y₂ᵢ₋₁ and Y₂ᵢ are independently selected from 0 to 20 contiguous amino acids, from 0 to 18 contiguous amino acids, from 0 to 15 contiguous amino acids, from 0 to 10 contiguous amino acids, from 0 to 5 contiguous amino acids, and/or wherein the total length of each Y₂ᵢ₋₁-Xᵢ-Y₂ᵢ is not more than 50 amino acids, not more than 45 amino acids, not more than 40 amino acids, not more than 35 amino acids, not more than 30 amino acids, not more than 25 amino acids.

In still other specific embodiments, the carrier polypeptide of the chimeric polypeptide as described herein has the following structure: (Y₂ᵢ₋₁-Xᵢ-Y₂ᵢ)ₙ, as defined above, wherein each Xᵢ corresponds to an amino acid sequence selected from the group consisting of:
V/I/L-X-Q-X-G-X-X-N/Q-X-A/V/I/L-X-X-X-Q (SEQ ID NO: 32), and wherein X means any amino acid.

As an alternative embodiment, the carrier polypeptide moiety of the chimeric polypeptide as described herein is selected from the group consisting of:
a polypeptide having an amino acid sequence of SEQ ID NO: 3,
a polypeptide that has at least 60% amino acid identity with SEQ ID NO: 3,
a fragment of a polypeptide having an amino acid sequence of SEQ ID NO: 3 or a fragment of a polypeptide that has at least 60% amino acid identity with SEQ ID NO: 3,
a polypeptide having an amino acid sequence selected from the group of SEQ ID NOS: 4-8, and
a polypeptide that has at least 60% amino acid identity with an amino acid sequence selected from the group of SEQ ID NOS: 4-8.

In particular, the disclosure provides embodiments that specifically relate to polypeptides whose sequence comprises or consists of the sequence of a naturally occurring bacterial CsgA polypeptide (as defined hereinbefore), as well as to variants and fragments of such naturally occurring bacterial CsgA polypeptide. As used herein, “variant” refers to any polypeptide or peptide differing from a naturally occurring polypeptide by amino acid insertion(s), deletion(s), and/or substitution(s), created using, e g., recombinant DNA techniques. In some embodiments, amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, tyrosine, asparagine, and glutamine. The positively charged (basic) amino acids include arginine, lysine and histidine. The negatively charged (acidic) amino acids include aspartic acid and glutamic acid. In some embodiments, cysteine is considered a non-polar amino acid. In some embodiments, insertions or deletions may range in size from about 1 to 20 amino acids, e.g., 1 to 10 amino acids. In some instances, larger domains may be removed without substantially affecting function. In certain embodiments, the sequence of a variant can be obtained by making no more than a total of 1, 2, 3, 5, 10, 15, or 20 amino acid additions, deletions, or substitutions to the sequence of a naturally occurring polypeptide. In some embodiments, not more than 1%, 5%, 10%, or 20% of the amino acids in a polypeptide or fragment thereof are insertions, deletions, or substitutions relative to the original polypeptide. In some embodiments, guidance in determining which amino acid residues may be replaced, added, or deleted without eliminating or substantially reducing activities of interest (i.e., retaining the capability to polymerize in vivo), may be obtained by comparing the sequence of the particular polypeptide with that of orthologous polypeptides from other organisms and avoiding sequence changes in regions of high conservation or by replacing amino acids with those found in orthologous sequences since amino acid residues that are conserved among various species may more likely be important for activity than amino acids that are not conserved. Thus, according to a particularly preferred embodiment of this disclosure, a variant should at least comprise the amino acid sequence V/I/L-X-Q-X-G-X-X-N/Q-X-A/V/I/L-X-X-X-Q (SEQ ID NO: 32), wherein X means any amino acid.

A “fragment” of a polypeptide refers to a subsequence of the polypeptide. Fragments may vary in size from as few as 10 amino acids to the length of the intact polypeptide, but are preferably at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150 amino acids in length. If desired, the fragment may be fused at either terminus to additional amino acids, which may number from 1 to 20, typically 50 to 100, but up to 250 to 500 or more. According to a preferred embodiment, a fragment as described herein is a “functional fragment,” which means a carrier polypeptide fragment retaining the capability to polymerize in vivo and in vitro. Thus, according to a particularly preferred embodiment of this disclosure, a fragment will at least comprise the amino acid sequence V/I/L-X-Q-X-G-X-X-N/Q-X-A/V/I/L-X-X-X-Q, wherein X means any amino acid.

According to a specific embodiment, the carrier polypeptide derived from a bacterial fiber subunit for displaying proteins is not derived from a subunit of flagella. According to other specific embodiments, the carrier polypeptide derived from bacterial fiber subunit for displaying proteins is not derived from a subunit of the chaperone/usher family pili, of Type IV pili, of Type III secretion-related organelles, or of Type IV secretion pili. According to yet another specific embodiment, the carrier polypeptide derived from bacterial fiber subunit as carrier protein for displaying proteins is not derived from a subunit of pili of Gram-positive bacteria.

### Passenger Polypeptides

In general, the nature of the passenger polypeptide is not critical to the disclosure, however, the size and structural features of the passenger polypeptide will determine whether a passenger polypeptide will be secreted by the Type VIII secretion system and attain its native fold. Particular embodiments of the passenger polypeptides that form part of the chimeric polypeptides are described further herein.

It will be understood that the passenger polypeptides differ from the carrier polypeptides as described hereinbefore, in that the passenger polypeptides of this disclosure do not comprise amino acid sequence VII/L-X-Q-X-G-X-X-N/Q-X-A/V/I/L-X-X-X-Q (SEQ ID NO: 32), wherein X means any amino acid. Accordingly, and in contrast with the carrier polypeptide, it will be clear that the passenger polypeptide in itself has no self-polymerizing properties. Whereas the carrier polypeptide moiety is meant for the passage through the type VIII secretion system and, if applicable, for the self-polymerizing property of the chimeric polypeptide, the passenger polypeptide moiety does not contribute to the formation of a polymeric structure. Instead, the passenger polypeptide moiety is co-secreted, and if applicable, can be displayed on the fiber surface as a functional protein and does not form part of the backbone of the fiber.

In particular embodiments, the passenger polypeptide that forms part of the chimeric polypeptide has an amino acid sequence of less than 800 amino acids, less than 700 amino acids, less than 600 amino acids, less than 500 amino acids, less than 400 amino acids, less than 350 amino acids, or less than 300 amino acids. Preferably, the passenger polypeptide that forms part of the chimeric polypeptide has an amino acid sequence of less than 250 amino acids, less than 200 amino acids, less than 150 amino acids, less than 100 amino acids, or less than 80 amino acids; and/or according to other preferred embodiments, the passenger polypeptide that forms part of the chimeric polypeptide has an amino acid sequence of at least 40 amino acids long, at least 50 amino acids, at least 60 amino acids, at least 80 amino acids, at least 100 amino acids, at least 110 amino acids, at least 120 amino acids, at least 150 amino acids, or at least 200 amino acids in length.

In other embodiments, the passenger polypeptide that forms part of the chimeric polypeptide has an amino acid sequence between 40 and 800 amino acids, between 50 and 700 amino acids, between 60 and 600 amino acids, between 80 and 350 amino acids, preferably between 100 and 300 amino acids, between 100 and 250 amino acids, between 110 and 250 amino acids, between 120 and 250 amino acids, or between 150 and 250 amino acids.

In other embodiments, the passenger polypeptide that forms part of the chimeric polypeptide preferably has particular structural features that depend on its folded dimensions. In particular, the passenger polypeptide as described herein has a transverse diameter of 4 nm or less, 3 nm or less, preferably 2.5 nm or less, when present in its folded conformation. In still other embodiments, the passenger polypeptide as described herein has at least four cysteines, preferably at least two cysteines that are involved in disulphide bridge formation.

Other particular embodiments of the passenger polypeptide relating to size and structural features are described in the Example section.

According to specific embodiments, the passenger polypeptide of the chimeric polypeptide is a binding domain (as defined hereafter). In particular, the passenger polypeptide of the chimeric polypeptide can also be a fusion of at least two binding domains, at least three binding domains, at least four binding domains. The at least two or more binding domains may be identical or not. According to other specific embodiments, the passenger polypeptide of the chimeric polypeptide is an enzyme. In particular, the passenger polypeptide of the chimeric polypeptide can also be a fusion of least two enzymes, at least three enzymes, or at least four enzymes. The at least two or more enzymes may be identical or not. Also envisaged are chimeric polypeptides of the disclosure, wherein the passenger polypeptide is fusion of at least one binding domain and at least one enzyme.

The term “binding domain,” as used herein, refers to a molecule that has the capability of interacting with a molecule of interest, for example, specific a target protein, a carbohydrate, a nucleic acid, a lipid, a small organic or small inorganic molecule. Within the scope of this disclosure, a binding domain is a polypeptide, more particularly, a protein domain. A protein domain is an element of overall protein structure that is self-stabilizing and often folds independently of the rest of the protein chain. Binding domains vary in length from between about 25 amino acids up to 500 amino acids and more. Many binding domains can be classified into folds and are recognizable, identifiable, 3-D structures. Some folds are so common in many different proteins that they are given special names. Non-limiting examples are binding domains selected from a 3- or 4-helix bundle, an armadillo repeat domain, a leucine-rich repeat domain, a PDZ domain, a SUMO or SUMO-like domain, an immunoglobulin-like domain, phosphotyrosine-binding domain, pleckstrin homology domain, src homology 2 domain, a lectin domain, and a metal-binding domain, amongst others. Antibodies are the natural prototype of specifically binding proteins with specificity mediated through hypervariable loop regions, so-called complementarity-determining regions (CDR). Although, in general, antibody-like scaffolds have proven to work well as specific binders, it has become apparent that it is not compulsory to stick strictly to the paradigm of a rigid scaffold that displays CDR-like loops. In addition to antibodies, many other natural proteins mediate specific high-affinity interactions between domains. Alternatives to immunoglobulins have provided attractive starting points for the design of novel binding (recognition) molecules. “Scaffold,” as used in this disclosure, refers to a protein framework that can carry altered amino acids or sequence insertions that confer binding to specific target proteins, carbohydrate, nucleic acids, lipids, and small organic or small inorganic molecules. Engineering scaffolds and designing libraries are mutually interdependent processes. In order to obtain specific binders, a combinatorial library of the scaffold has to be generated. This is usually done at the DNA level by randomizing the codons at appropriate amino acid positions, by using either degenerate codons or trinucleotides. A wide range of different non-immunoglobulin scaffolds with widely diverse origins and characteristics are currently used for combinatorial library display. Some of them are comparable in size to an scFv of an antibody (about 30 kDa), while the majority of them are much smaller. Modular scaffolds based on repeat proteins vary in size depending on the number of repetitive units. Frequently, when generating a particular type of binding domain using selection methods, combinatorial libraries comprising a consensus or framework sequence containing randomized potential interaction residues are used to screen for binding to a molecule of interest, such as a protein, a carbohydrate, a nucleic acid, a lipid, a small organic or small inorganic molecule.

A non-limiting list of examples comprise binding domains or scaffolds based on the human 10th fibronectin type III domain, binders based on lipocalins, binders based on SH3 domains, binders based on members of the knottin family, binders based on CTLA-4, T-cell receptors, neocarzinostatin, carbohydrate binding module 4-2, tendamistat, kunitz domain inhibitors, PDZ domains, Src homology domain 2 (SH2), scorpion toxins, insect defensin A, plant homeodomain finger proteins, bacterial enzyme TEM-1 beta-lactamase, Ig-binding domain of Staphylococcus aureus protein A, E. coli colicin E7 immunity protein, E. coli cytochrome b562, designed ankyrin-repeat domains (DARPins), alphabodies, lipopeptides (e.g., pepducins), anticalins, and affibodies.

Also included as binding domains are compounds with a specificity for a given target protein, cyclic and linear peptide binders, peptide aptamers, multivalent avimer proteins or small modular immunopharmaceutical drugs, ligands with a specificity for a receptor or a co-receptor, protein binding partners identified in a two-hybrid analysis, binding domains based on the specificity of the biotin-avidin high affinity interaction, and binding domains based on the specificity of cyclophilin-FK506 binding proteins. Also included are lectins with an affinity for a specific carbohydrate structure. Also included are metal-binding domains with an affinity for a specific metal.

For more examples, see also, e.g., Gebauer and Skerra, 2009; Skerra, 2000; Starovasnik et al., 1997; Binz et al., 2004; Koide et al., 1998; Dimitrov, 2009; Nygren et al. 2008; and WO2010/066740.

In one embodiment, the passenger polypeptide is a binding domain that is derived from an immunoglobulin. Preferably, the passenger polypeptide according to the disclosure is a binding domain that is derived from an antibody or an antibody fragment. Non-limiting examples of immunoglobulin-based binding domains include antibodies, heavy chain antibodies (hcAb), single domain antibodies (sdAb), minibodies, the variable domain derived from camelid heavy chain antibodies (VHH or nanobodies), the variable domain of the new antigen receptors derived from shark antibodies (VNAR), and engineered CH2 domains (nano-antibodies).

The term “antibody” (Ab) refers generally to a polypeptide encoded by an immunoglobulin gene, or a functional fragment thereof, that specifically binds and recognizes an antigen, and is known to the person skilled in the art. The term “antibody” is meant to include whole antibodies, including single-chain whole antibodies, and antigen-binding fragments. In some embodiments, antigen-binding fragments may be antigen-binding antibody fragments that include, but are not limited to, Fab, Fab′ and F(ab′)2, Fd, single-chain Fvs (scFv), single-chain antibodies, disulfide-linked Fvs (dsFv) and fragments comprising or consisting of either a VL or VH domain, and any combination of those or any other functional portion of an immunoglobulin peptide capable of binding to the target antigen. The term “antibodies” is also meant to include heavy chain antibodies, or functional fragments thereof, such as single domain antibodies, more specifically, immunoglobulin single variable domains such as VHHs or nanobodies, as defined further herein.

In a particular embodiment, the passenger polypeptide is a binding domain that is an immunoglobulin single variable domain that comprises an amino acid sequence comprising four framework regions (FR1 to FR4) and three complementarity-determining regions (CDR1 to CDR3), preferably according to the following formula (1):

FR1-CDR1-FR2-CDR2-FR3-CDR3-FR4 (1),

or any suitable fragment thereof (which will then usually contain at least some of the amino acid residues that form at least one of the complementarity-determining regions).

Binding domains comprising four FRs and three CDRs are known to the person skilled in the art and have been described, as a non-limiting example, in Wesolowski et al. (2009, Med. Microbiol. Immunol. 198:157). Typical, but non-limiting, examples of immunoglobulin single variable domains include light chain variable domain sequences (e.g., a V_{L} domain sequence), or heavy chain variable domain sequences (e.g., a V_{H} domain sequence), which are usually derived from conventional four-chain antibodies. Preferably, the immunoglobulin single variable domains are derived from camelid antibodies, preferably from heavy chain camelid antibodies, devoid of light chains, and are known as V_{H}H domain sequences or nanobodies (as described further herein). Thus, in a preferred embodiment, the passenger polypeptide is a nanobody. In another embodiment, the passenger polypeptide is a fusion of at least two nanobodies, at least three nanobodies, or more.

The term “nanobody” (Nb), as used herein, refers to the smallest antigen binding fragment or single variable domain (V_{H}H) derived from naturally occurring heavy chain only antibody and is known to the person skilled in the art. They are derived from heavy chain only antibodies, seen in camelids (Hamers-Casterman et al. 1993; Desmyter et al. 1996). The single variable domain heavy chain antibody is herein designated as a Nanobody or a V_{H}H antibody. Nanobody™ and Nanobodies™ are trademarks of Ablynx NV (Belgium).

The delineation of the CDR sequences (and, thus, also the FR sequences) is based on the IMGT unique numbering system for V-domains and V-like domains (Lefranc et al. 2003). Alternatively, the delineation of the FR and CDR sequences can be done by using the Kabat numbering system as applied to V_{H}H domains from Camelids in the article of Riechmann and Muyldermans (2000). As will be known by the person skilled in the art, the immunoglobulin single variable domains, in particular, the nanobodies, can, in particular, be characterized by the presence of one or more Camelidae hallmark residues in one or more of the framework sequences (according to Kabat numbering), as described, for example, in WO 08/020079, on page 75, Table A-3, incorporated herein by reference.

### Linker Moiety

According to another embodiment, the chimeric polypeptide encoded by the recombinant nucleic acid molecule as described above further comprises a linker moiety. In particular, the carrier polypeptide and passenger polypeptide as comprised in the chimeric polypeptide as described hereinabove, can be fused to each other either directly or through a linker moiety. The nature and/or length of the linker moieties are not critical to the disclosure. According to particular embodiments, the linker is selected from a stretch of between 0 and 20 identical or non-identical units, wherein a unit preferably is an amino acid, but can also be a monosaccharide, a nucleotide or a monomer (in the case where a chimeric polypeptide would be synthetically designed, see further herein).

Typically, “linker molecules” or “linkers” are peptides of 0 to 20 amino acids length and are typically chosen or designed to be unstructured and flexible. For instance, one can choose amino acids that form no particular secondary structure. Or, amino acids can be chosen so that they do not form a stable tertiary structure. Or, the amino acid linkers may form a random coil. Such linkers include, but are not limited to, synthetic peptides rich in Gly, Ser. Thr, Gin, Glu or further amino acids that are frequently associated with unstructured regions in natural proteins (Dosztanyi et al. 2005). Non-limiting examples include (GS)₅ or (GS)₁₀.

Preferably, the amino acid linker sequence is relatively short, has a low susceptibility to proteolytic cleavage and does not interfere with the biological activity of chimeric polypeptide. According to specific embodiments, an amino acid linker sequence is a peptide of between 0 and 20 amino acids, between 0 and 10 amino acids, particularly between 0 and 5 amino acids. Particularly envisaged sequences of short linkers include, but are not limited to, PPP, PP or GS.

For certain applications, it may be advantageous that the linker molecule comprises or consists of one or more particular sequence motifs. For example, at least one proteolytic cleavage site can be introduced into the linker molecule such that the displayed passenger protein can be released after surface display. Useful cleavage sites are known in the art, and include a protease cleavage site such as Factor Xa cleavage site having the sequence IEGR (SEQ ID NO: 74), the thrombin cleavage site having the sequence LVPR (SEQ ID NO: 75), the enterokinase cleaving site having the sequence DDDDK (SEQ ID NO: 76), or the PreScission cleavage site LEVLFQGP (SEQ ID NO: 77).

Non-limiting examples of suitable linker sequences are also described in the Example section.

### Signal Peptide Moiety

According to a preferred embodiment, the chimeric polypeptide encoded by the recombinant nucleic acid molecule as described above further comprises a signal peptide moiety.

In bacteria, a signal peptide (as defined herein) is a prerequisite for proteins to be translocated across the cytoplasmic membrane to the periplasm in Gram-negatives (diderms) or extracellular space in Gram-positives (monoderms). Suitable signal peptides will typically depend on the host cell and the protein to be translocated, and are known by the person skilled in the art. For example, signal peptides may be chosen such that they direct the proteins to the Sec secretion system. Other signal peptides will direct the proteins to the Tat (the Twin arginine translocase) secretion pathway. Thus, depending on the host cell and the protein to be translocated, the skilled person can easily select a suitable signal peptide, for example, by using the SignalP webserver (on the World Wide Web at cbs.dtu.dk/services/SignalP/), which predicts the presence and location of signal peptides and there cleavage sites in amino acid sequences from different organisms, including Gram-positive prokaryotes, Gram-negative prokaryotes, and eukaryotes.

Non-limiting examples of signal peptide sequences include OmpA, PelB, LamB, SurA, DsbA, TolB, and PhoA leader sequences.

According to specific embodiments, signal peptides of naturally occurring CsgA polypeptides may also be used, for example, SEQ ID NO: 30. Non-limiting examples of suitable signal peptides are also described in the Example section.

### Vectors

This disclosure also provides for a vector comprising the recombinant nucleic acid molecule as described hereinbefore.

The vector generally contains elements required for replication in a prokaryotic host system. Such vectors, which include plasmid vectors and viral vectors such as bacteriophage, are well known and can be purchased from a commercial source (e.g., Promega, Madison Wis.; Stratagene, La Jolla Calif.; GIBCO/BRL, Gaithersburg Md.) or can be constructed by one skilled in the art. The construction of expression vectors and the expression of a polynucleotide in transformed or transfected cells involves the use of molecular cloning techniques also well known in the art (see Sambrook et al., in Molecular Cloning: A Laboratory Manual (Cold Spring Harbor Laboratory Press 1989); Current Protocols in Molecular Biology (eds., Ausubel et al.; Greene Publishing Associates, Inc., and John Wiley & Sons, Inc. 1990 and supplements)).

### Host Cells

This disclosure also provides for a host cell comprising the vector or recombinant nucleic acid molecule as described hereinbefore. It will be appreciated that in some embodiments, the recombinant nucleic acid molecule as described herein can be integrated in the genome of the host cell. Within the context of this disclosure, preferably host cells of bacterial origin are transformed with any of the recombinant nucleic acid sequences or vectors as described herein. In particular, the bacterial host cells as provided herein may be Gram-positive bacterial host cells or Gram-negative bacterial host cells, which are terms commonly used in the art for the classification of Bacteria. Essentially, any bacterial host cell can be chosen. When a Gram-negative bacterial host cell is chosen, the secretion machinery responsible for the assembly of curli fibers (as defined hereinbefore) needs to be present (also called Type VIII secretion system or nucleation-precipitation pathway), which minimally encompasses a CsgG protein, and preferably also the accessory proteins CsgE or CsgF. Further, within the context of this disclosure, the bacterial host cell is engineered so that the expression of genes encoding the proteins of the Type VIII secretion system and the expression of the recombinant nucleic acid molecule encoding the chimeric polypeptide is synchronized. A typical way of achieving this is by using an appropriate set of (inducible) promoters. The choice of a promoter will typically depend on the nature of the host cell. The choice further depends on the desired temporal expression of a particular fusion protein as described herein. In this regard, promoters include constitutive promoters, inducible promoters and repressible promoters. According to specific embodiments, the conditions for inducing or repressing any of the promoters are selected from the group consisting of metabolic, or stress, or pH, or temperature, or drug-inducing or repressing conditions, or other inducing or repressing conditions. Examples of suitable promoters are described in “Useful proteins from recombinant bacteria” in Gilbert et al., 1980, Scientific American 242:74-94; and in Sambrook et al., 1989, Molecular Cloning: A Laboratory Manual.

In one specific embodiment, the bacterial host cell is a Gram-negative bacterial host cell. In accordance with a more systematic phylogenetic classification, particularly envisaged are bacteria belonging to the phylum Proteobacteria and Bacteroidetes, which constitute a major group of Gram-negative bacteria, including the genera Escherichia, Salmonella, Klebsiella, Shigella, Enterobacter, and other Enterobacteriaceae, Pseudomonas, Moraxella, Helicobacter, Stenotrophomonas, Bdellovibrio, acetic acid bacteria, Legionella and numerous others. Suitable bacterial hosts include Enterobacteria, such as Escherichia coli, Shigella dysenteriae, Klebsiella pneumoniae, and the like. Mutant cells of any of the above-mentioned bacteria may also be employed, as is also illustrated in the Example section.

In general, a Gram-negative bacterial host cell endogenously expresses the csgBAC and csgDEFG operons under the control of their natural promoter. In some embodiments, the csgBAC and/or csgDEFG operons, and/or csgA, csgB, CsgC, CsgD, CsgE, CsgF and/or CsgG individually or a combination of any thereof, can be exogenously expressed in the host cell on a plasmid under the control of its natural promoter or, alternatively, under the control of an inducible promoter. A variety of inducible promoters can be compatible with expression of one or more of the genes of the csgBAC and/or csgDEFG operons, and are known in the art. It will be appreciated that a cell that expresses such plasmids may also express endogenous copies of csgBAC and/or csgDEFG. In some embodiments, the endogenous copies of csgBAC and/or csgDEFG are mutated or deleted. According to one particular embodiment, the bacterial host cell does not endogenously express csgA.

For certain applications, it may be advantageous to express the recombinant nucleic acid molecules encoding the chimeric polypeptides of the disclosure in a non-pathogenic bacterial host cell or an attenuated strain.

According to other embodiments, the bacterial host cell encompasses a Gram-positive host cell comprising such a recombinant nucleic acid sequence or vectors as described herein. The host cell is, for instance, a lactic acid bacterium, preferably selected from Lactococcus lactis, Bacillus subtilis, Streptococcus pyogenes, Staphylococcus epidermis, Staphylococcus gallinarium, Staphylococcus aureus, Streptococcus mutans, Staphylococcus warneri, Streptococcus salivarius, Lactobacillus sakei, Lactobacillus plantarum, Carnobacterium piscicola, Enterococcus faecalis, Micrococcus varians, Streptomyces OH-4156, Streptomyces cinnamoneus, Streptomyces griseoluteus, Butyrivibrio fibriosolvens, Streptoverticillium hachijoense, Actinoplanes linguriae, Ruminococcus gnavus, Streptococcus macedonicus, and Streptococcus bovis, amongst others.

Upon expression and subsequent secretion from the host cell, the chimeric polypeptides may self-assemble into curli fibers by virtue of the polymerizing nature of the carrier polypeptide. Carrier polymerization encompasses a conformational transition from a disordered to a cross-β structure and is nucleated by pre-existing cross-β fibers (including curli or curli fragments) or a nucleation polypeptide exposed on the same bacterial host cell surface or a foreign surface (which can be another bacterial surface or an artificial surface). Notably, where surface display on the producing host cell is envisaged, any of the mentioned bacterial strains endogenously expresses (e.g. Gram-negative bacteria) or can be transformed with (e.g. Gram-positive bacteria) the genes needed to nucleate the chimeric polypeptide protein. Thus, according to the embodiment where polymerization occurs on the producing host cell, the bacterial host cell comprising the vector or recombinant nucleic acid molecule as described hereinbefore, also expresses a nucleation polypeptide, for example, CsgB. Alternatively, in the embodiment where the polymerization occurs on or near another bacterial surface, the corresponding other bacterial cell needs to present a nucleation polypeptide, for example, CsgB or pre-existing cross-β fibers, including curli or curli fragments. In the embodiment where the polymerization occurs on an artificial surface (as defined further herein), the artificial surface is activated with a nucleation agent, for example, surfaces activated with CsgB, a cross-β fiber, CsgA or a nucleating CsgA peptide to trigger the polymerization of chimeric polypeptides secreted from a bacterial host cell.

In general, the nucleic acid molecules as provided herein can be transferred into any host cell by conventional methods, which vary depending on the type of cellular host (see, generally, Maniatis et al., Molecular Cloning: A Laboratory Manual (Cold Spring Harbor Press, 1982)). Selection of the appropriate vector system, regulatory regions and host cell is common knowledge within the level of ordinary skill in the art. It is expected that vectors, promoters, and the like can be similarly utilized and modified to permit expression of the chimeric polypeptides of the disclosure in other bacterial hosts. Replicability of the replicon in the bacteria is taken into consideration when selecting bacteria for use in the methods of the disclosure. Methods suitable for the maintenance and growth of bacterial cells are all well known.

Of particular interest and also envisaged herein, is a library of host cells, comprising a plurality of host cells according to the disclosure, wherein each member of the library displays at its cell surface a different passenger polypeptide. The library is particularly suitable to screen for agents that will bind to the displayed protein (as described further herein).

This disclosure also encompasses a composition comprising one or more chimeric polypeptides encoded by one or more recombinant nucleic acid molecules as described hereinabove, whereby the passenger polypeptide of each chimeric polypeptide in the composition is a functionally active polypeptide. According to one embodiment, the composition is a fiber. The composition may be attached to a surface, in particular, a cell surface or an artificial surface (as described further herein).

Within the context of this disclosure, it is envisaged to use the composition for detecting and/or capturing of a substance, such as a protein, an organic or inorganic compound, a heavy metal, or a pollutant. Or alternatively, it is envisaged to use the composition for the chemical and/or enzymatic conversion of a substance, such as a protein, an organic or inorganic compound, a heavy metal, or a pollutant.

Within the context of this disclosure, the capture of a substance (such as a protein, an organic or inorganic compound, a heavy metal, or a pollutant) encompasses its binding to the passenger polypeptide moiety fused to the carrier protein moiety, that form part of the chimeric polypeptide as described hereinbefore. In a particular embodiment, the chimeric polypeptide is displayed on a bacterial cell, which is freely suspended in solution, is adsorbed onto a solid or gel-like surface or a suspended particle, or is present in a bacterial biofilm. In a particular embodiment, the chimeric polypeptide is displayed directly on a solid surface, a suspended organic, anorganic or mixed organic—anorganic particle, or an organic or inorganic gel-like matrix. Capture of the substance entails the exposure of a solution holding the substance to the capture material, by suspension of the capture material to the substance solution or by contact of the capture medium and the substance solution in a continuous flow process. The substances are non-covalently or covalently bound by the capture material and thus retained from the solution carrying the substances. In the context of a chemical or enzymatic conversion by the capture material, the substances are modified and the resulting products are released back to the carrying solution.

One further aspect of this disclosure relates to a method for producing a chimeric polypeptide in the extracellular medium of a host cell culture, the method comprising the steps of:
a) providing a host cell that is genetically engineered to express a CsgG protein, or variant or fragment thereof, and a chimeric polypeptide comprising:
   i. a carrier polypeptide comprising an amino acid sequence V/I/L-X-Q-X-G-X-X-N/Q-X-A/V/I/L-X-X-X-Q (SEQ ID NO: 32), wherein X means any amino acid,
   ii. a passenger polypeptide of 50 amino acids or more, and
   iii. optionally, a linker that couples a) to b), and
b) culturing the host cell of a) under suitable conditions to express and secrete the chimeric polypeptide into the extracellular medium,
   whereby the CsgG protein, or variant or fragment thereof, and the chimeric polypeptide are expressed concomitantly, and whereby the passenger polypeptide of the chimeric polypeptide is maintained as a functionally active polypeptide after secretion.

In one embodiment, the method further comprises the step of isolating the chimeric polypeptide from the culture medium.

Further embodiments of chimeric polypeptides and suitable host cells and expressing conditions are described above and also apply here.

According to one aspect, this disclosure also envisages a method of producing a functionalized fiber, the method comprising the steps of:
a) providing a host cell that is genetically engineered to express a chimeric polypeptide comprising:
   i. a carrier polypeptide comprising an amino acid sequence V/I/L-X-Q-X-G-X-X-N/Q-X-A/V/I/L-X-X-X-Q (SEQ ID NO: 32), wherein X means any amino acid,
   ii. a passenger polypeptide of 50 amino acids or more, and
   iii. optionally, a linker that couples a) to b), and
b) culturing the host cell of a) under suitable conditions to express the chimeric polypeptide, and
c) allowing the chimeric polypeptide to polymerize into a fiber, whereby the passenger polypeptide is displayed as a functionally active polypeptide.

In one particular embodiment, the above-described method further comprises the step of isolating the expressed chimeric polypeptide from the cell before step c). In the alternative, the expressed chimeric polypeptide is secreted from the cell. According to a specifically preferred embodiment of the above-described method, the passenger polypeptide of the chimeric polypeptide is maintained as a functionally active polypeptide after secretion or isolation.

According to one embodiment, step c) of the above-described method occurs on or near the extracellular surface of the same or another host cell. According to another embodiment, step c) occurs on or near an artificial surface. An artificial or synthetic surface may be a bead, a slide, a chip, a plate, or a column. More particularly, the artificial surface may be particulate (e.g., beads or granules) or in sheet form (e.g., membranes or filters, glass or plastic slides, microtiter assay plates, dipstick, or capillary devices), which can be flat, pleated, or hollow fibers or tubes.

In still another embodiment, step c) of the above-described method occurs in solution, for example, without limitation, in the extracellular medium of the producing bacterial host cell.

Further embodiments of chimeric polypeptides and suitable host cells and expressing conditions are described above and also apply here.

### EXAMPLES

### Example 1

### Secretion of Heterologous Sequences by the Curli Outer Membrane Translocator CsgG

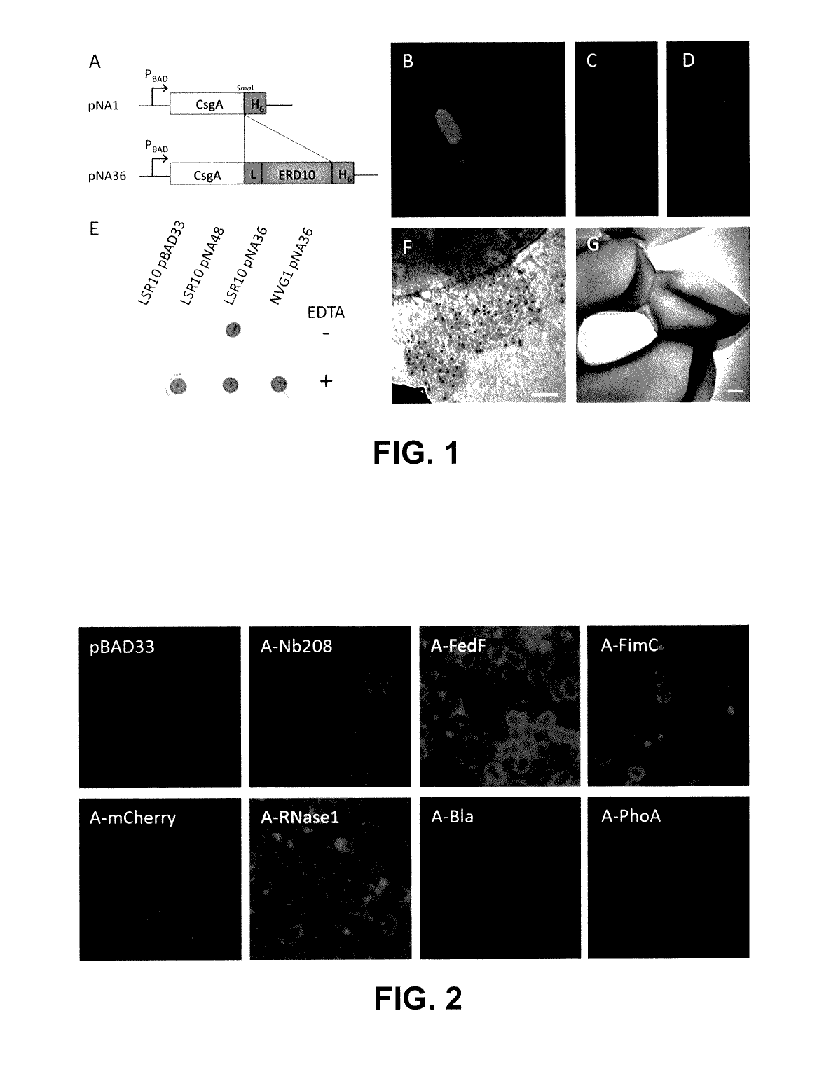

In previous studies, short peptide stretches (9 to 16 residues in length) within the major Salmonella curli subunit, AgfA, have been successfully replaced by different T-cell epitopes (White et al., 1999; White et al., 2000; Huang et al., 2009; Meng et al., 2010). To further explore whether there are sequence specific or structural restrictions for passage through the CsgG transmembrane pore, a more extensive heterologous sequence was fused to the CsgA C-terminus. Because CsgA is believed to be in an extended conformation during secretion, ERD10 (early response to dehydration), a 260-residue intrinsically disordered protein from plants (Kovacs et al., 2008), was used as passenger sequence. ERD10 was C-terminally 6×His-tagged and fused by its N-terminus to the major curli subunit CsgA. This fusion was cloned in the pBAD33 vector under the control of the P_{BAD} promoter, resulting in plasmid pNA36 (FIG. 1, Panel A). Expression was confirmed in E. coli DH5α cells by Western blotting using antibodies against the C-terminal Histidine tag. In order to investigate secretion and curli production, the csgA-ERD10 fusion was expressed in LSR10 cells (i.e., MC4100ΔcsgA (Chapman et al., 2002)) under curli-inducing conditions to assure physiological levels of the curli secretion machinery through chromosomal expression of csgG, csgE, csgF, csgB and csgC. The secretion of CsgA-ERD10 by LSR10 (pNA36) was first confirmed by immunofluorescence (IF) microscopy on whole cells using an antibody directed to the 6×His-tag. Bacteria producing the fusion protein revealed a clear fluorescent halo surrounding the cells (FIG. 1, Panel B), while no fluorescence was detected for the pBAD33 empty vector control (FIG. 1, Panel C) or bacteria transformed with a plasmid encoding 6×His-tagged ERD10 with the CsgA N-terminal SEC signal peptide only (pNA48) (i.e., without the N22 sequence for CsgG targeting) (FIG. 1, Panel D). Whole cell dot blots of LSR10 (pNA48) are positive for anti-6×His staining only upon OM permeabilization with EDTA and lysozyme (FIG. 1, Panel E), demonstrating cell envelope integrity and stable expression of ERD10-6×His in the periplasm. Furthermore, no extracellular CsgA-ERD10 was detected in a csgA csgG double knockout strain (NVG1: MC4100ΔcsgAΔcsgG) (FIG. 1, Panel E). Detection of 6×His ERD10 by whole cell dot blot or IF is thus specific to its fusion to CsgA and its surface exposure in a secretion process that is dependent on the CsgG transporter (FIG. 1, Panels B and E). In conjunction with the pericellular fluorescence observed by IF, anti-6×His immunogold Transmission EM (TEM) analysis on LSR10 (pNA36) confirmed that CsgA-ERD10 accumulated into cell-associated extracellular material (FIG. 1, Panel F) that was absent in the pBAD33 negative control (FIG. 1, Panel G).

### Example 2

### Size and Structural Limitations for Type VIII Secretion Substrates

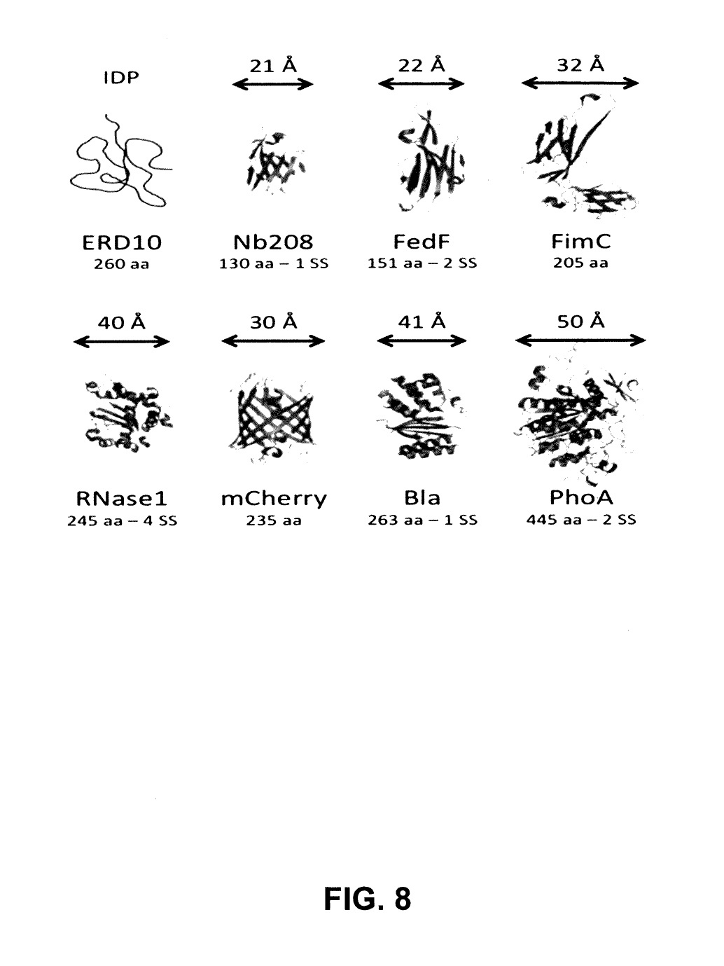

Next, a systematic investigation was carried out as to whether CsgA-targeted transport through the Type VIII secretion pathway was possible for folded passenger proteins. For this purpose, a range of well-characterized proteins or domains was selected, differing in size, secondary structure composition and disulfide bond content (FIG. 8). The chosen passengers either naturally occur or are well produced in the periplasm, confining the challenge of their extracellular display to the last step in transport, the OM translocation through the pore formed by CsgG. In this way, a llama single domain antibody (Nb208), RNase1, periplasmic chaperone FimC, β-lactamase (Bla), fimbrial lectin domain FedF₁₅₋₁₆₅, alkaline phosphatase PhoA, as well as mCherry were C-terminally fused to CsgA, analogously to the ERD10 construct, resulting in plasmids pNA15, pNA29, pNA30, pNA31, pNA32, pNA33, and pNA34, respectively (Table 4).

Anti-6×His immunoblot analysis of E. coli DH5α cells transformed with the different plasmids revealed that after a 45-minute induction in liquid LB medium at 37° C., the cultures produced the respective recombinant fusion proteins. Longer induction, however, caused lysis of the bacterial cells. This toxicity was gauged to be due to the absence of the curli assembly machinery, expression of which is restricted to prolonged growth (48 hours) on solid medium at low temperature (Olsen et al., 1989). Accordingly, in further experiments, induction of the fusion proteins was delayed by growth on two-layered glucose/arabinose agar plates at room temperature in order to synchronize with curli-promoting conditions. Except for CsgA-PhoA, delayed induction of the CsgA-fusions no longer resulted in cell lysis, suggesting that the Csg protein machinery protected cells from the cytotoxic species and/or CsgA fusion proteins were now transported outside the cell. As for the ERD10 fusion, anti-6×His antibodies were used in IF to get an initial observation of the display of the different fusion proteins. FIG. 2 shows Nb208, FedF, FimC and RNase1 fusions clearly exhibited a green fluorescence associated with the bacterial cell envelopes. In the case of CsgA-Bla or CsgA-mCherry, only diffuse or punctuate fluorescence was observed, respectively, while LSR10 cells harboring the csgA-PhoA fusion construct or the pBAD33 negative control did not bind the anti-6×His antibodies.

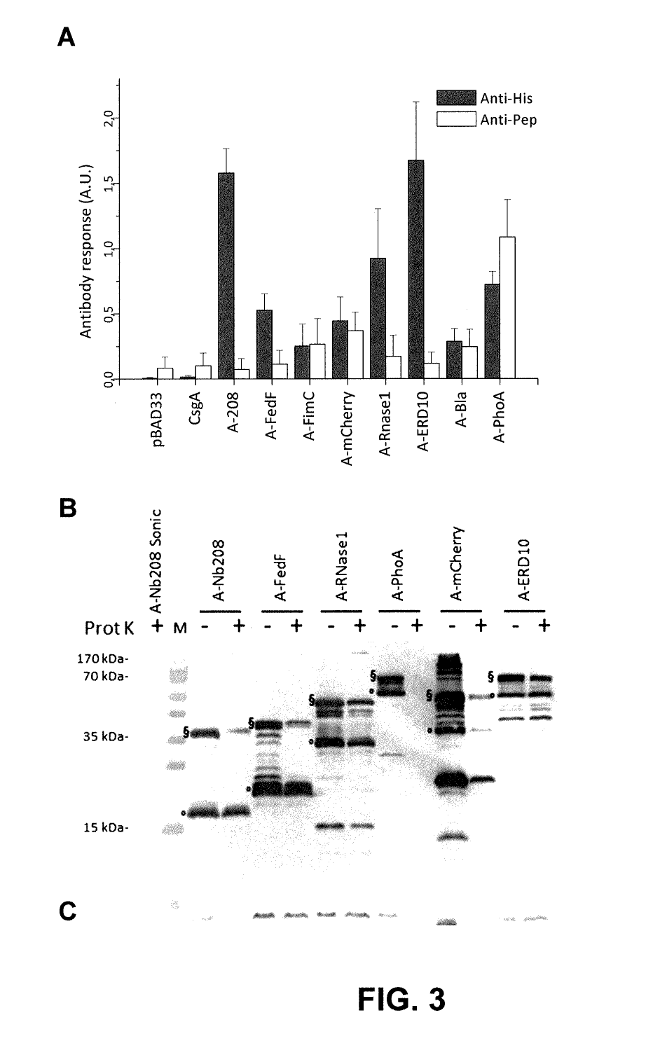

To acquire a more quantitative measure of secretion, the extracellular exposure of the fusion's C-terminal 6×His-tag was monitored using whole cell ELISA (FIG. 3, Panel A). As a parallel control for OM integrity, accessibility of the murine layer was assessed with a monoclonal anti-peptidoglycan antibody (Veiga et al., 1999). Induced cells were scraped from agar plates, resuspended in PBS to an OD_{6OOnm} of 1.0 prior to coating. To further ascertain that anti-6×His and anti-peptidoglycan ELISA reads were proportional to the amount of cells coated, an anti-E. coli antiserum was used for normalization. The anti-6×His antibodies bound selectively to cells expressing the fusion proteins and did not label WT CsgA or the vector control (FIG. 3, Panel A). Strong anti-6×His ELISA signals were found for CsgA-Nb208 and CsgA-ERD10, followed by intermediate signals for CsgA-RNase1, CsgA-PhoA, CsgA-FedF and CsgA-mCherry. CsgA-FimC and CsgA-Bla1 showed low, though significant levels of 6×His detection (p<0.001) (FIG. 3, Panel A). For CsgA-Nb208, CsgA-FedF, CsgA-RNase1 and CsgA-ERD10, anti-peptidoglycan signals were at WT CsgA or vector control levels (p>0.05), showing these fusions did not perturb OM integrity and that 6×His detection consequently represents fusion proteins secreted to the bacterial surface (FIG. 3, Panel A). In contrast, however, ELISA on LSR10 cells expressing CsgA-PhoA, CsgA-mCherry, CsgA-FimC and CsgA-Bla showed raised peptidoglycan detection compared to vector control or WT CsgA (p<0.05), indicating a breach of the cell envelope. Therefore, any anti-6His responses for these fusion products cannot be regarded as proportionally representative of their Type VIII-mediated secretion. Instead, IF and ELISA detection of apparent surface-associated material could also come from non-specific leakage to the extracellular surface and/or stem from antibody intrusion into the periplasm. It should be noted that for CsgA-PhoA, the anti-6×His response in IF and whole cell ELISA does not correspond. This discrepancy could be due to harsher conditions in the ELISA, leading to more OM permeabilization, or to better binding of the released proteins to the ELISA plate than to the poly-L-lysin on the glass slides.

To obtain a further measure of secretion efficiency, the proportion of intra-versus extracellular material was monitored for a select number of CsgA-fusions (CsgA-ERD10, CsgA-Nb208, CsgA-FedF, CsgA-RNase1, CsgA-PhoA and CsgA-mCherry) by means of their protease susceptibility. As a control for cell envelope integrity, protease sensitivity of the endogenous, periplasmically located oxidoreductase DsbA was monitored in parallel. For all tested constructs, anti-6×His Western analysis of whole cell lysates showed the presence of both the full-length CsgA-fusions as well as bands corresponding to the passenger proteins only, stemming from fusions that had lost their N-terminal CsgA portion due to proteolitic processing (FIG. 3, Panel B). For LSR10 cells expressing CsgA-ERD10, CsgA-Nb208, CsgA-FedF or CsgA-RNase1, prior treatment with extracellularly added proteinase K leads to the partial breakdown of the CsgA-fusion products, while bands corresponding to DsbA or the passenger only remained untouched. Instead, when cells were first ruptured by brief sonication, proteinase K treatment lead to the full breakdown of any 6×His-tagged products (FIG. 3, Panel B). Thus, the lack of proteinase K exposure of DsbA or the passenger fragments demonstrates that for the ERD10, Nb208, FedF and RNase1 fusions, the OM barrier is maintained and that, therefore, the proportion of CsgA-fusion in protease-treated versus non-treated samples is representative of the fraction CsgA-fusion that is secreted to the cell surface. Furthermore, the protection from protease K for the passenger fragments that lost the N-terminal CsgA sequence shows that secretion of the fusions to the cell surface is specific to the presence of the latter. FIG. 3, Panel B, reveals that for Nb208 and FedF, the majority of the CsgA-fusion product was surface-exposed, whereas for CsgA-RNase1 and CsgA-ERD10, approximately half or one-third of the fusion remained intracellular, respectively. In the case of CsgA-mCherry and CsgG-PhoA, proteinase K treatment lead to the loss of both the full fusion proteins and the passengers, as well as of the periplasmic DsbA, reiterating the observations by ELISA that expression of these fusions leads to a breach of the cell envelope.

### Example 3

### Secreted CsgA-Bound Passenger Proteins can Attain their Native Fold

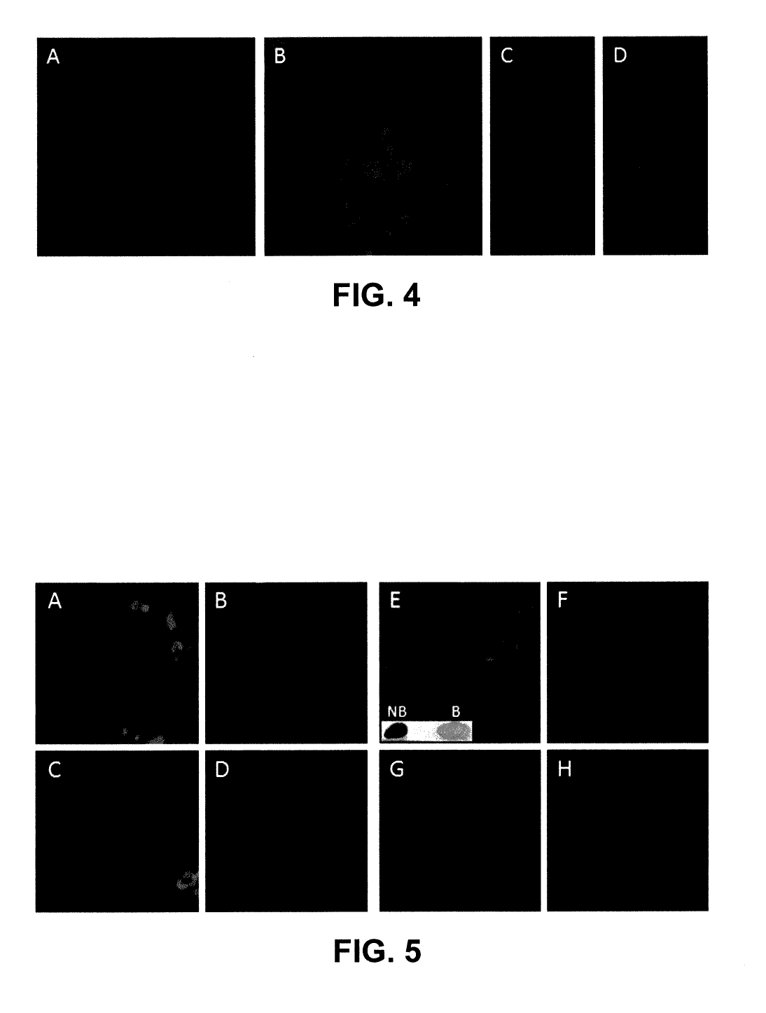

Next, examination was carried out as to whether the CsgA-fused passenger proteins can attain their native fold following Type VIII-mediated secretion. Nb208 is a GFP-binding single domain antibody, a property that was used as reporter of its structural conformation on the bacterial cell surface. Like the CsgA-ERD10 fusion, LSR10 cells harboring plasmid pNA15 (csgA-Nb208) showed a marked pericellular fluorescence during anti-6×His immunostaining of the fusion protein in the surface-exposed material (FIG. 4, Panel A). A green fluorescent halo was also seen when purified GFP was added to induced LSR10 (pNA15) cells (FIG. 4, Panel B). No binding of GFP was seen in control experiments with either wild-type CsgA or vector control (data not shown). Furthermore, LSR10 (pNA18) (pCA0747) cells, producing a periplasmic form of Nb208 and CsgA fused to a lysozyme-binding nanobody (NbCabLys3) (Desmyter et al., 1996), did not bind GFP (FIG. 4, Panel C). This showed GFP binding was specific to expression of CsgA-Nb208 and permitted, in addition to anti-peptidoglycan ELISA or proteinase K assays shown above, independent control for potential OM permeabilization caused by the artificial CsgA-nanobody fusion proteins. Only upon prolonged induction (4 days), green punctuate staining could be detected in about 3% of an LSR10 (pNA18) (pCA0747) culture (FIG. 4, Panel D), corresponding to cells that lost their membrane integrity. This internal punctuate staining was clearly distinct from the halo seen around intact cells expressing the csgA-Nb208 fusion (FIG. 4, Panel B) and occurred mainly in elongated cells. Thus, GFP is recruited to the bacterial cell surface by its binding to cell-bound CsgA-Nb208 fusion, demonstrating that following Type VIII secretion, Nb208 is able to attain its native fold and is displayed in an accessible and active conformation on the surface of the bacterial cells.

### Example 4

### CsgG-Mediated Transport is Compatible with Passage of Non-Linear Polypeptides

For CsgG, biochemical and EM structural studies point to the formation of a 2-nm wide oligomeric channel that transports its CsgA substrate in an extended, unfolded conformation (Chapman et al., 2002; Robinson et al., 2006). The observations above illustrate that when fused to CsgA, heterologous proteins can be accepted for secretion, but that secretion efficiencies are dependent on the folded dimensions of the passenger protein. Strikingly, whereas CsgA-FimC and CsgA-mCherry fusions show poor or no specific secretion, the similarly sized, but intrinsically unfolded, protein ERD10 is efficiently secreted and incorporated into surface-exposed CsgA-fusions. This suggests that rather than the linear size, the folding of the passenger protein prior to secretion and the size of its tertiary structure form the blockage for CsgG-mediated secretion. Notably, the transverse diameter of the passenger proteins that showed poor secretion ranges from 3.2 to 5 nm (FIG. 8), exceeding the CsgG channel diameter estimated by EM (Chapman et al., 2002; Robinson et al., 2006). On the other hand, Nb208 and FedF comprise Ig-like domains with a transverse diameter of about 2.5 nm, similar in size to the reported CsgG channel diameter and raising the possibility that the passenger moieties of CsgA-Nb208 and CsgA-FedF fusions are secreted in a folded conformation.

Nanobodies contain two cysteines that form a disulfide bridge between framework β-strands 1 and 3. In E. coli, disulfide bridge formation and isomerization is DsbA/DbsC-catalyzed and takes place in the periplasm (Nakamoto and Bardwell, 2004; Messens and Collet, 2006). In the case of autotransporters, the introduction of disulfide-bound “knots” in the passenger domain has been used to study the transport mechanism (Klauser et al., 1990). Such knotted passengers obstructed translocation unless disulfide bond formation in the E. coli periplasm was prevented, either by the addition of β-mercaptoethanol (2-ME) to the growth medium or by the use of an E. coli dsbA mutant (Klauser et al., 1990). Similarly, it was reasoned that the presence of an oxidized disulfide in surface-exposed CsgA-Nb208 fusion would indicate the substrate passed the CsgG transporter in a non-linear conformation. To confirm whether Nb208 in surface-exposed CsgA-Nb208 had an oxidized disulfide, Nb208 activity in a mutant where one of the two cysteines was mutated to serine (CsgA-Nb208^{C22S}) was assessed. Although extracellular material containing CsgA-Nb208^{C22S} was similarly displayed on the bacterial surface, as evidenced by anti-6×His IF staining (FIG. 5, Panel A), it no longer bound extracellular GFP (FIG. 5, Panel B). Thus, surface-displayed CsgA-Nb208 is present in its oxidized form. To assess whether disulfide formation is a result from the periplasmic Dsb oxidative pathway or rather stems from spontaneous oxidation on the extracellular surface, CsgA-Nb208 was expressed in the E. coli dsbA knockout strain MD1. Though CsgA-Nb208 was efficiently transported, it was unable to bind GFP (FIG. 5, Panels C and D), demonstrating that in absence of DsbA, secreted CsgA-Nb208 is found in a reduced and inactive conformation. Thus, disulfide formation in Type VIII dependent secretion of the CsgA-Nb208 fusion is DsbA-dependent and occurs prior to secretion.

The fimbrial lectin domain FedF₁₅₋₁₆₅ contains two disulfide bonds that stabilize its R-sandwich fold and an elongated loop near its receptor binding site (Moonens et al., 2012). Using an anti-FedF nanobody that selectively recognizes a conformational epitope in the FedF sugar binding site (Nb231) (FIG. 5, Panel E insert), examination was performed as to whether the extracellular FedF₁₅₋₁₆₅ is presented in a folded, functional conformation. Induced LSR10 (pNA32) bacterial cells, producing the CsgA-FedF fusion protein, stained bright green with FITC-labeled Nb231 (FIG. 5, Panel E). A drastically weaker fluorescence signal was seen when the displayed FedF was reduced by treating cells with DTT or 2-ME prior to IF (FIG. 5, Panel F). No fluorescent labeling was observed in control cells, producing only CsgA, a CsgA-FimC fusion or FedF₁₅₋₁₆₅ in the periplasm (data not shown). The CsgA-FedF fusion was still transported to the outside in the E. coli dsbA knockout strain MD1 (FIG. 5, Panel G), but Nb231 no longer recognized FedF (FIG. 5, Panel H). Thus, surface-displayed FedF is functional and contains its canonical disulfide bonds, which oxidize prior to secretion. Together, these observations indicate Nb208 and FedF adopt a non-linear, possibly fully folded conformation prior to CsgG-mediated secretion.

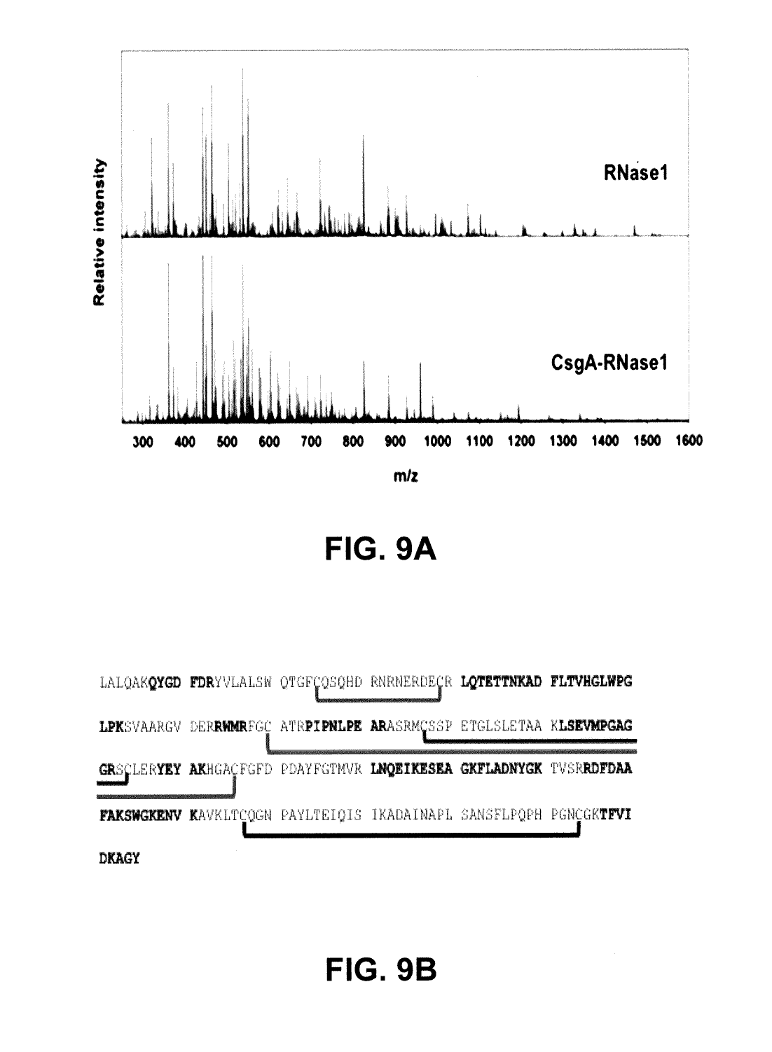

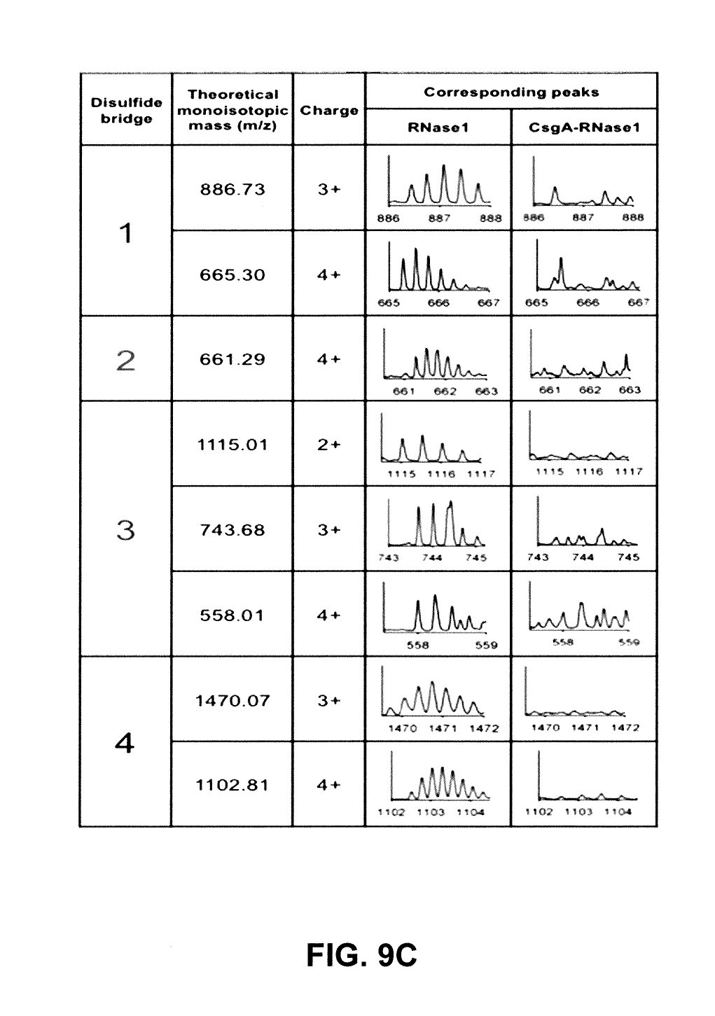

Finally, though RNase1 gets partially secreted (FIGS. 2 and 3), the folded protein has a diameter of 40 Å, similar to that for FimC, mCherry and Bla (FIG. 8), which showed no or very poor CsgG-dependent secretion. RNase1 contains four cysteine bridges for which the correct pair-wise disulfide bonding is essential for RNase activity (Messens et al., 2007). Under non-DsbA/DsbC catalyzed oxidation/isomerization, the canonical disulfide pairing is scrambled, leading to an inactive enzyme. Therefore, the SDS-insoluble fraction was isolated from LSR10 (pNA29) and the formation of the correct disulfide pairs was investigated by mass spectrometry after trypsin digestion. For an RNase1 control produced and purified from the periplasm, the four canonical disulfide bridges were detected amongst the peptide fragments (FIGS. 9A-9C). However, for RNase1 fused to CsgA, none of the predicted disulfide-bonded peptides were clearly detected, whereas predicted non-cysteine-containing RNase1 peptides were (FIGS. 9A-9C). The spectra also show an absence of peaks corresponding to the unpaired cysteine-containing peptides, showing that cysteines in RNase1 fused to CsgA were oxidized, but in a randomized pairing (FIGS. 9A-9C). Although the SDS-insoluble fraction does not necessarily derive solely from extracellular material, together, these data indicate that the SDS-stable fraction of the CsgA-RNase1 fusion that was secreted to the bacterial surface had not attained its Dsb-catalyzed disulfide bridge conformation and native protein folding prior to passage through the curli secretion machinery.

### Example 5

### Structural Nature of Cell Surface-Bound CsgA-Fusions

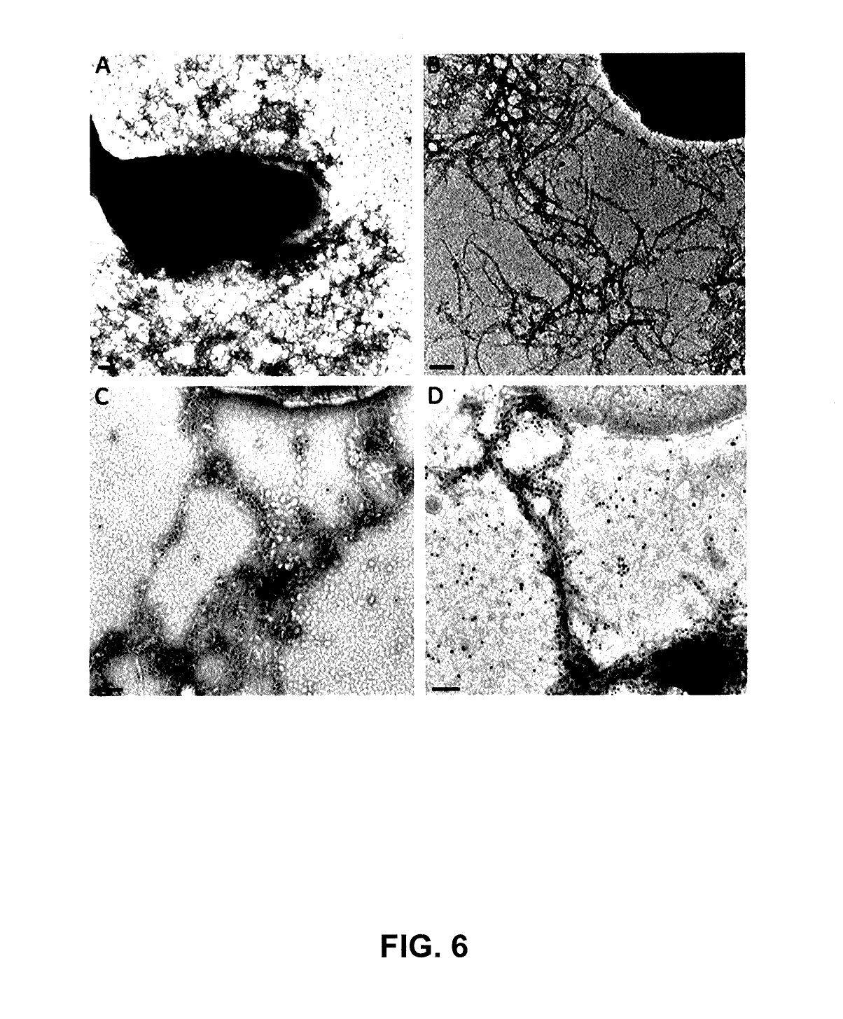

Secreted native CsgA is found as fibrillar filaments that show the physical characteristics of amyloids and can be seen as negatively staining fibrils of 6-12 nm by EM (Chapman et al., 2002). TEM analysis of LSR10 (pNA15) showed an abundant extracellular matrix associated with the cells (FIG. 6, Panel A). The morphology of the secreted material, however, differed from the ordered fibrils seen for native curli in MC4100 (FIG. 6, Panel B) and appeared, in the most part, as a dense aggregate (FIG. 6, Panel A). Besides this positively staining dense matrix, negatively staining filamentous threads could also be observed (FIG. 6, Panel C), and were found to incorporate the CsgA-Nb208 fusion on the basis of Ni-NTA-gold staining. Nevertheless, the fibrillous structure of these threads was not as prominent and instead appeared more thin and flexible compared to the fibrils found for native CsgA (FIG. 6, Panel B) or an isogenic strain expressing CsgA-6×His (LSR10 (pNA1)) (FIG. 6, Panel D).

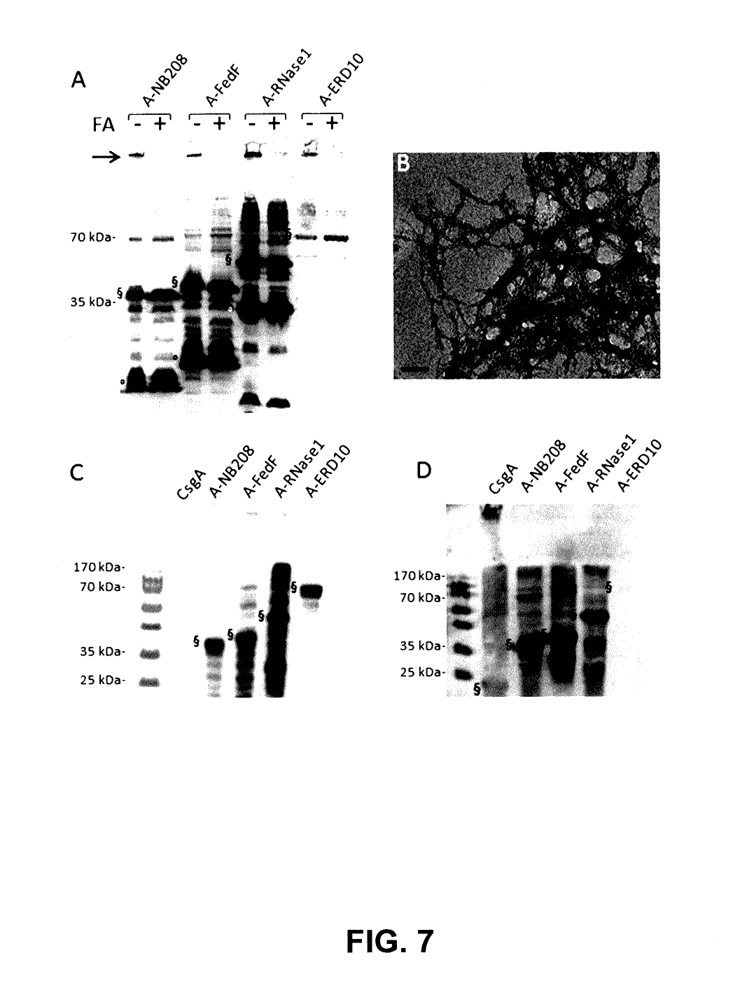

Native curli fibrils are resistant to heating in SDS and require formic acid (FA) treatment for depolymerization, unlike amorphous or colloidal protein aggregates or other filamentous organelles such as pili and flagella (Chapman et al., 2002; Fronzes et al., 2008). To more quantitatively monitor to what extent the matrix of secreted CsgA-fusions contained fibrillar, SDS-insoluble material versus SDS-soluble aggregates, cell lysates were analyzed by SDS-PAGE with or without FA treatment. For all secreted fusions, anti-6×His Western blotting showed the presence of SDS-insoluble material that did not migrate into the stacking gel unless treated with FA (FIG. 7, Panel A). Though not fully quantitative, comparison of non-treated versus FA-treated samples showed that the dominant fraction of the different CsgA-fusion proteins was present as SDS-soluble material (FIG. 7, Panel A), in line with the main morphology observed by TEM in case of CsgA-Nb208 (FIG. 6, Panel A). The SDS-insoluble fractions of the different cultures were isolated through two consecutive boiling steps in a 10% SDS buffer, visualized by TEM (FIG. 7, Panel B) or dissolved with formic acid and analyzed by SDS-PAGE and anti-6×His or anti-CsgA Western blotting to reveal their protein composition (FIG. 7, Panels C and D). TEM analysis of the SDS-insoluble fraction of LSR10 (pNA15) showed clear fibrils reminiscent of curli and distinct of the dense positively staining matrix seen to form the major fraction of secreted CsgA-Nb208 fusion surrounding the cells. Blots developed with Anti-6×His showed that the SDS-insoluble fractions contained the species running at the molecular weight expected for the various intact CsgA-fusions as well as a number of proteolytic fragments that lost part of the N-terminal CsgA sequence (FIG. 7, Panels C). It was unclear whether the latter species were part of the fibrillous material or resulted from acid hydrolysis during formic acid treatment of the samples. Development with an anti-CsgA antibody revealed that the intact CsgA-fusion proteins represented the dominant CsgA-containing species in the fibrillar fractions (FIG. 7, Panel D). Notably, although for CsgA-ERD10 anti-6×His staining confirmed the presence of the intact fusion inside fibrillar material, this species showed very weak staining with the anti-CsgA antibody. The reason for this reduced Anti-CsgA staining is unclear.

### Example 6

### Defining Minimal CsgA Sequences for Functional Display of Heterologous Polypeptides

In order to define minimal CsgA domains necessary for transport and functional display of heterologous polypeptides, several CsgA repeat (R1 up to R5) deletions were made in the CsgA-flex-NB208-His fusion construct. In practice, deletion of CsgA repeats in the CsgA-NB208 fusions were carried out by “outwards” PCR on pNA15, using primer combinations DelR5FW and DelR1Rev, DelR5FW and DelR2Rev, DelR5FW and DelR3Rev, DelR5FW and DelR4Rev, DelR5FW and DelR5, DelR1FW and DelR1Rev, or Rev DelN22FW and DelN22Rev, giving rise to pNA20, pNA21, pNA22, pNA23, pNA24, pNA25, pNA26, respectively (see Table 4). Δ1-5 (expressed from plasmid pNA20) represents the removal of CsgA repeats R1 to R5, leaving only the N22 sequence fused to NB208 in the mature protein. Δ2-5, Δ3-5, and Δ4-5 stand for the deletions of R2 to R5, R3 to R5 and R4 to R5, respectively, and their corresponding coding plasmids are pNA21, pNA22 and pNA23. Δ5, Δ1 and ΔN22 symbolize CsgA-NB208 fusions lacking R1, R5 or N22 and are coded on plasmids pNA24, pNA25 and pNA26. Except for ΔN22, all NB208 fusions above retain the N22 signal sequence.

Additionally, it was investigated whether the single CsgA repeats, without N22 present, would still be able to display NB208 at the cell surface and form fibers. Therefore, chimeric constructs of only one repeat of CsgA (without N22) fused to NB208 were made by “outwards” PCR. Starting from pNA21, pNA22, pNA23, pNA24. or pNA18 with primer combinations Rev DelN22FW and DelN22Rev, DelN22Rev and Del R1FW, DelN22Rev and R3 Fw, DelN22Rev and R4 Fw or DelN22Rev and R5 Fw, respectively, this PCR resulted in plasmids pSB1, pSB2, pSB3, pSB4 and pSB5 (see Table 4).

The presence of the CsgA repeat deletions seemed to have no influence on the level of fusion protein produced in DH5α, as determined in Western blotting (data not shown).

To test whether cells expressing the protein fusions were able to produce curli, LSR10 cells harboring the different deletion constructs were grown on Congo red agar under curli-expressing conditions. Curli production was monitored by the degree of colony staining and further examined using TEM. To evaluate whether the CsgA deletion-fused passenger protein was properly folded, the intrinsic property of NB208 to bind GFP was exploited using fluorescence microscopy.

On Congo red indicator plates, LSR10 cells expressing the different fusions looked pink to red, depending on the deletion (FIG. 13). The fact that all fibers still bound to Congo red, indicates that the different proteins still polymerized and adapted a β-sheet-rich structure.

Further, the necessity of N22 for transport through CsgG was evaluated. Although the N22 is said to be the secretion signal for CsgG, LSR10 cells harboring a CsgA-NB208 fusion lacking this N22 (ΔN22) still secreted curli fusion products, as determined by Congo red binding (FIG. 13) and TEM (FIG. 10, Panel A). Furthermore, since GFP binding could be observed around induced LSR10 (pNA26) cells (FIG. 10, Panel B), NB208 was functionally displayed on the bacterial surface. This suggests that the other repeats of CsgA can also provide a curli-specific secretion signal, independent of the presence or absence of N22.

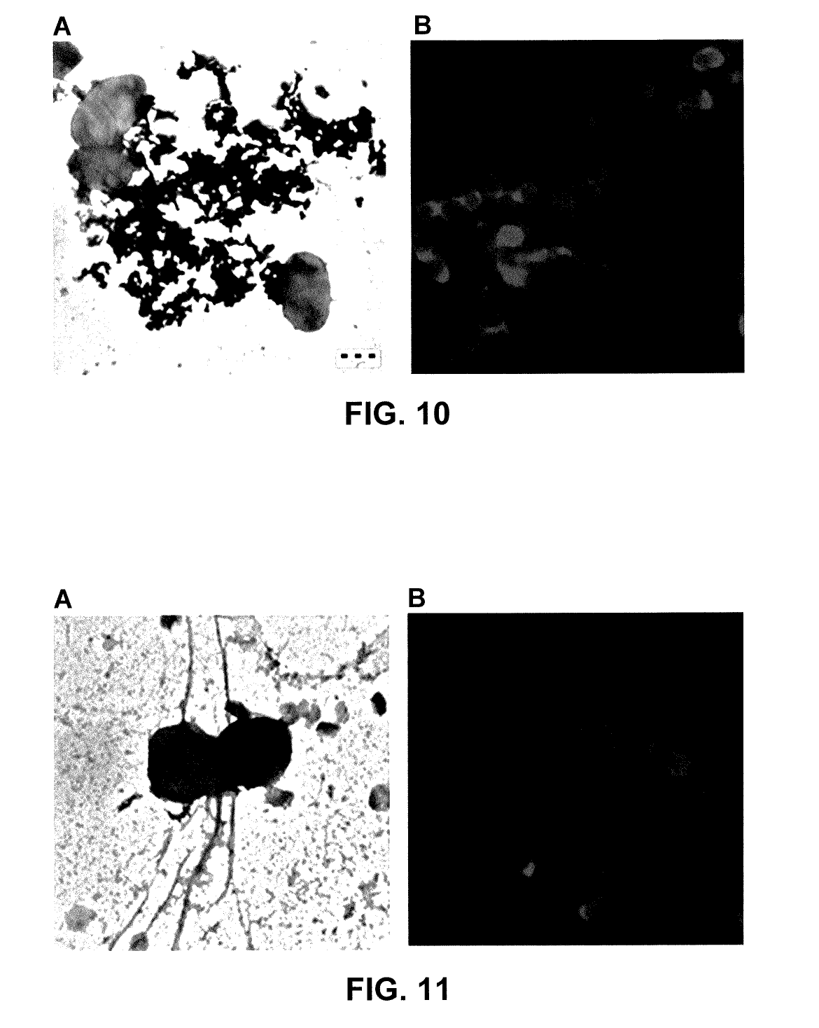

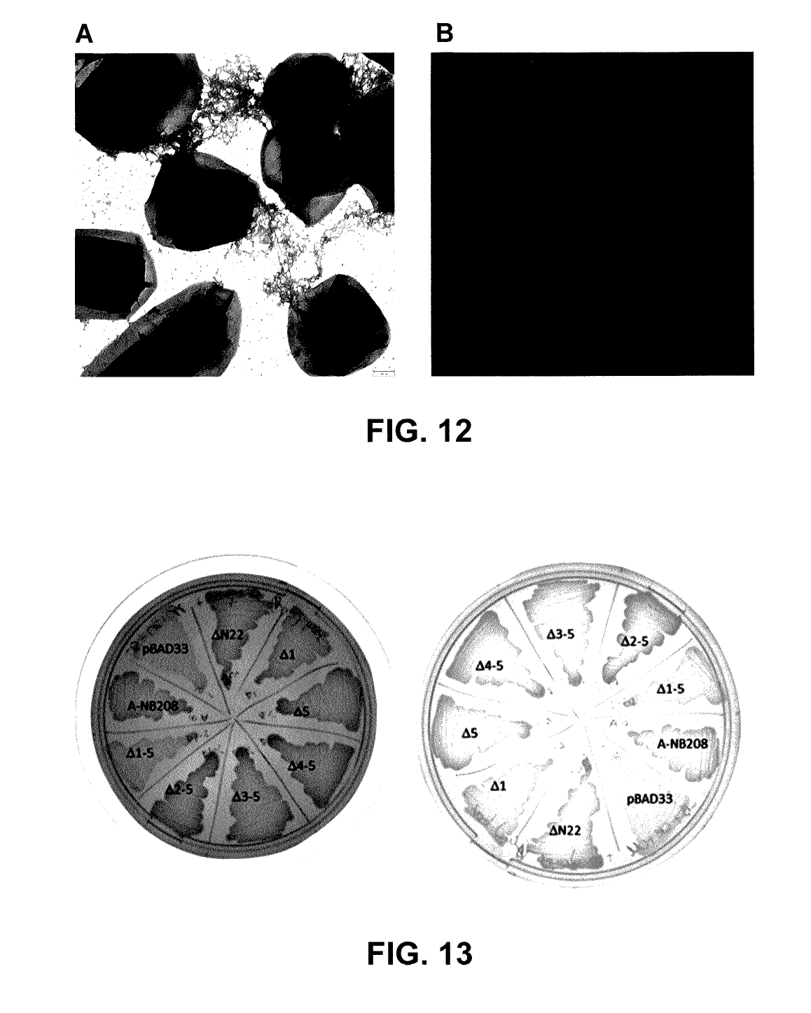

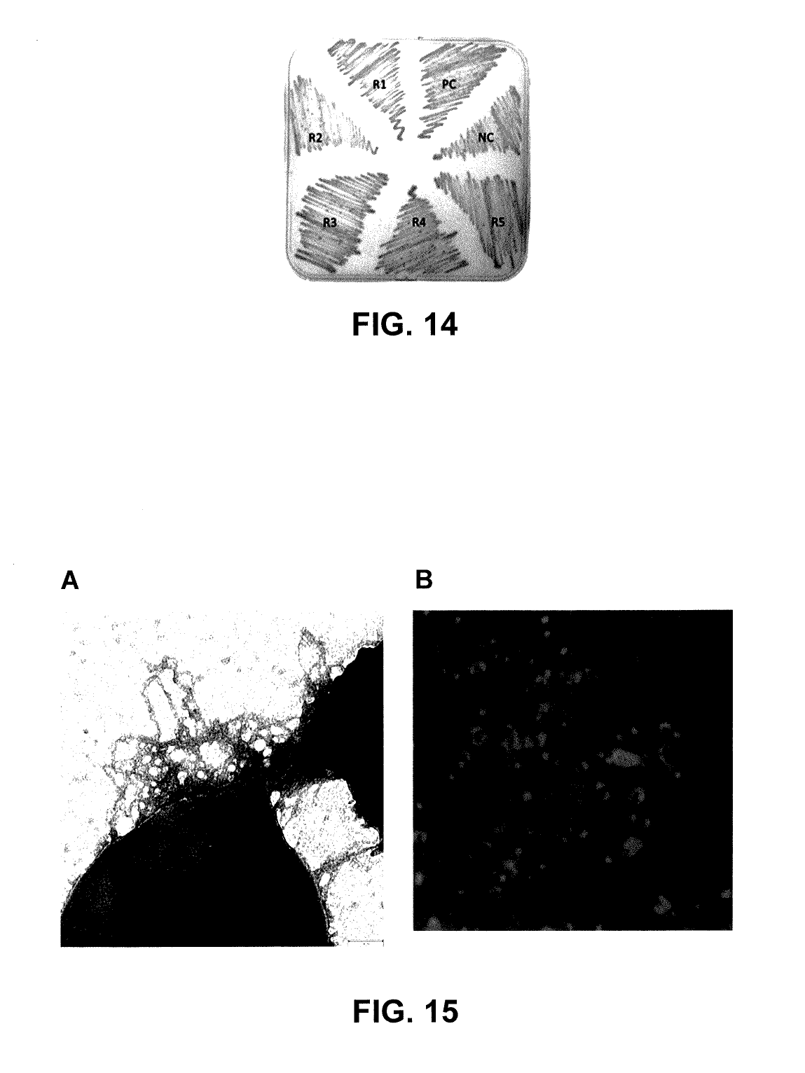

Further, the necessity of the different CsgA repeats R1 to R5 for transport through CsgG was evaluated. LSR10 (pNA21), i.e., Δ2-5, produced colonies that reacted stronger with Congo red than wild-type MC4100 or LSR10 cells expressing the CsgA-NB208 fusion protein. LSR10 (pNA25), i.e., Δ1, bound Congo red to the same extent as the intact CsgA-NB208 fusion. However, for both Δ2-5 and Δ1, TEM showed that curli were less abundant than in the wild-type and were architecturally distinct as they tended to arrange into thick bundled fibers (FIG. 11, Panel A, and FIG. 12, Panel A). Both Δ2-5 and Δ1 produced curli on which the functional NB208 is presented, as cells expressing these constructs could bind externally added GFP (FIG. 11, Panel B, and FIG. 12, Panel B). LSR10 (pSB2), i.e., R2-NB208, produce slightly red colonies on Congo red indicator plates, but in TEM, fibers are clearly visible (FIG. 15, Panel A). Furthermore, these fibers display NB208 in a functional conformation that is able to bind GFP (FIG. 15, Panel B). These experiments indicate that not all the repeats are necessary for transport through CsgG and that single repeats, even in the absence of N22, can transport heterologous proteins through CsgG and that heterologous proteins are functionally displayed in fibers.

### Example 7

### Display of Hybrid Fibers Composed of Multiple Different Fusion Proteins

For some applications, it might be beneficial to display different polypeptides, with different functionalities, in the same fiber structure. This can be achieved by co-expressing two or more different fusion polypeptides in the same bacterial cell, or by mixing two or more populations of cells, each harboring one single fusion polypeptide. For other applications, a combination of wild-type CsgA and CsgA fusion polypeptides might be beneficial. CsgA can then be co-expressed on a different or on the same plasmid as the fusion polypeptide in a csgA knockout strain. Otherwise, the chromosomal copy of csgA can also be used. As demonstrated in Example 6, the minimal amyloid repeating sequence of CsgA is sufficient as carrier polypeptide for display. Combinations of wild-type CsgA and one or more repeating units fused to different polypeptides are, therefore, also possible.

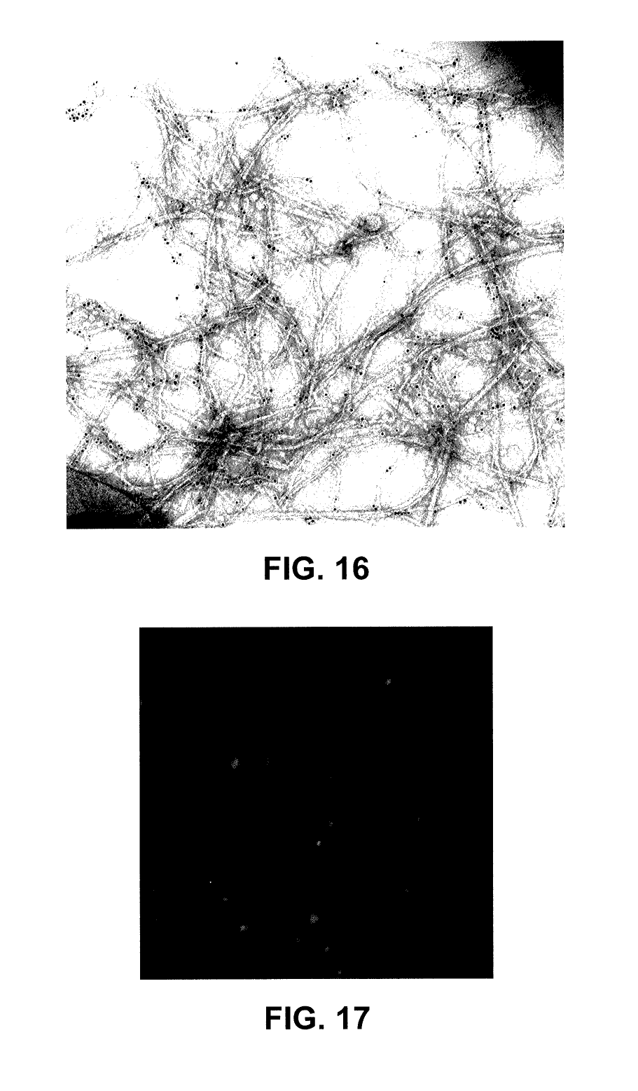

As a proof-of-principle, MC4100 (pNA15) was used to display hybrid fibers composed of a CsgA-Nb208 fusion and native CsgA as a spacer (see FIG. 16). As shown in FIG. 16, mixed nature curli fibers can be formed comprising protomers of both CsgA-Nb and native CsgA, and with a morphology identical to wild-type fibers or fibers of the CsgA-fusion protein alone. The CsgA-NB208 fusion protein is present in these mixed fibers, as Ni-NTA gold beads are binding the his-tag of the fusion protein (FIG. 16).

### Example 8

### Display of the NB208 Fusion in Salmonella

Curli are also produced by Enterobacteriaceae other than E. coli (Barnhart et al., 2006). For example, in Salmonella spp., these curli fibers are called thin aggregative fimbriae (Tafi) (Collinson et al., 1991). To investigate the broadening of the host cell range, the CsgA-flex-NB208-His fusion (pNA15) was expressed in Salmonella enterica serovar Typhimurium c3000. Exogenously added GFP bound specifically to induced c3000 (pNA15), proving functional curli display across species (FIG. 17).

### Example 9

### Secretion and Fiber Formation of CsgA-Fusion Proteins by Gram-Positive Bacteria

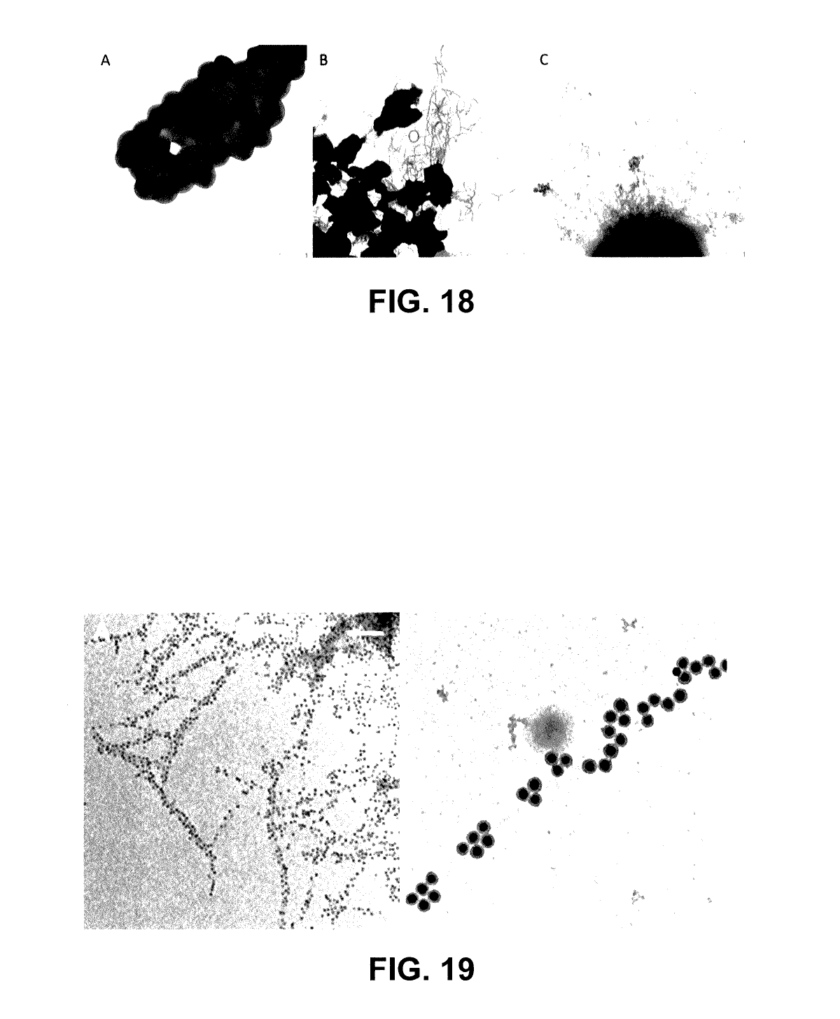

Further testing was performed to ascertain if, apart from Gram-negative bacteria, Gram-positive bacteria could also be used to secrete CsgA fusion proteins that are able to form functional fibers. In this way, the problems caused by extensive folding of the passenger in the periplasm can be circumvented. For this purpose, the csgA-His, csgA-flex-NB208-His and csgA-flex-Bla-His fusions were cloned in vectors compatible with secreted expression in Lactococcus lactis, under the control of different constitutive promoters. Anti-histidine Western blotting proved the correct fusion proteins were produced in the supernatant (data not shown). For the Bla fusions, the correct folding of the Bla moiety was shown by growth of L. lactis harboring the CsgA-Bla fusion under the control of five different promoters (i.e., P9, Cplc, LacA1, SplA, P43, respectively, pEXP435, pEXP436, pEXP437, pEXP438, pEXP439) on agar plates containing ampicillin. All five promoters yielded enough CsgA-Bla to provide resistance to ampicillin (data not shown). In transmission electron microscopy (TEM), fibers were visible on L. lactis(pEXP424) and L. lactis(pEXP437) cells, harboring the csgA-NB208 or csgA-Bla fusions, respectively (FIG. 18, Panels B and C), while in the negative control, L. lactis cells were bald (FIG. 18, Panel A). The His-tag of CsgA-Bla was further detectable with Ni-NTA gold, proving the presence of the fusion protein in these fibers (FIG. 18, Panel C).

### Example 10

### In Vitro Grown Hybrid CsgA Fibers Display the NB208 Fusion Protein in its Active Conformation

For some applications, it might be useful to grow functional hybrid fibers in vitro. As proof of this concept, CsgA-NB208-His was produced cytoplasmically in E. coli BL21DE3 cells and afterwards purified via nickel affinity chromatography. The ability to form amyloid fibers in vitro was demonstrated by ThT fluorescence and TEM (data not shown). Ni-NTA gold (5 nm) binding to CsgA-NB208-His fibers grown in vitro for 1 week shows the intact fusion is present in these fibers (FIG. 19, Panel A). GFP coupled to nanogold further binds specifically to the CsgA-NB208-His fibers, indicating NB208 is functionally folded and able to bind its target, GFP (FIG. 19, Panel B).

### Example 11

### In Vitro Grown CsgA Fibers Coupled on a Solid Surface

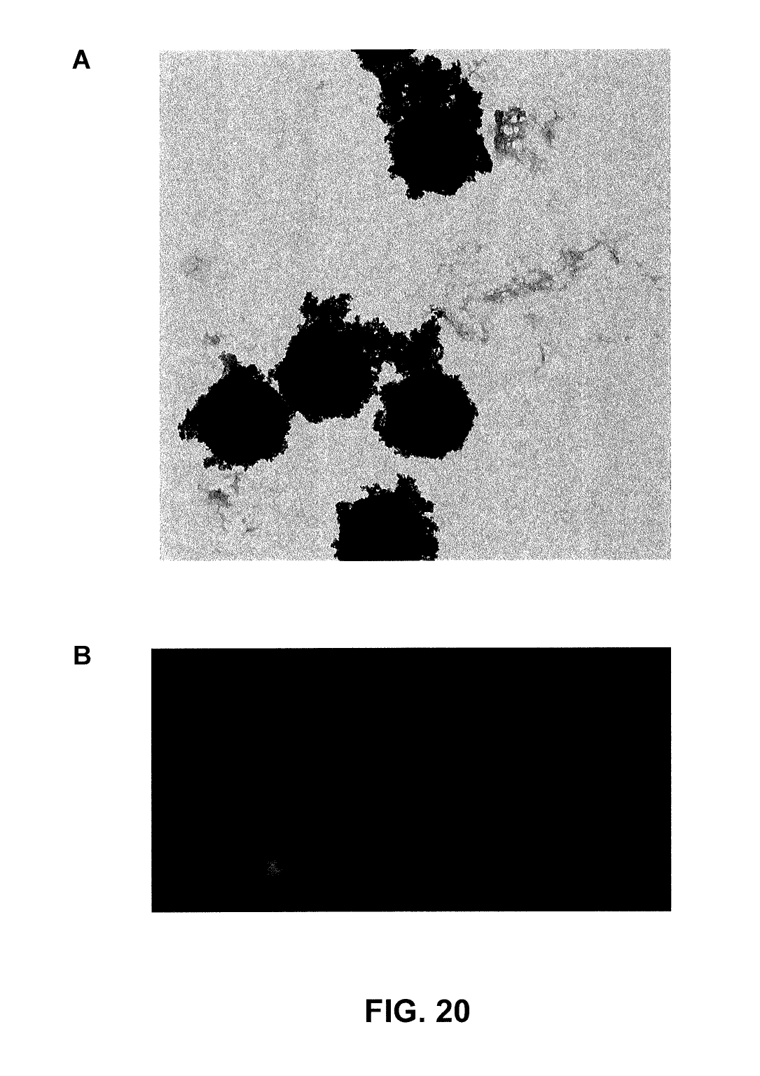

For some biotechnological applications, the display of proteins is desired on non-biotic surfaces. Here, CsgA fibers were coupled onto a synthetic surface, namely a magnetic bead. CsgA-6×His was expressed without signal peptide in E. coli BL21DE3 cells and, after production, purified via nickel affinity chromatography. In vitro produced CsgA-6×His fibers (formed in a concentrated CsgA-6×His solution in MES buffer over a three-week period) were sonicated to obtain nuclei, which were covalently coupled to carboxylate-modified magnetic microparticles via the direct EDAC procedure. These activated beads were added to a solution of purified CsgA-His. The coupling of the in vitro grown fibers to the particles was demonstrated by TEM and IF microscopy using an antibody directed to the 6×His-tag (FIG. 20, Panels A and B). A fluorescent halo surrounding the magnetic beads was seen, indicating stable binding of the CsgA-6×His proteins to the particles (FIG. 20, Panel B). Activated microparticles are incubated in the presence of purified CsgA-NB208-His, to allow the growth of hybrid CsgA-NB208 fibers.

### Material and Methods to the Examples

### Bacterial Growth Conditions

Bacteria were grown at 37° C. on solid Lysogeny Broth (LB) (Bertani, 2004) or in liquid LB medium supplemented with ampicillin (100 mg l⁻¹) or chloramphenicol (25 mg l⁻¹) when required. To induce curli expression, bacteria were grown for 48 hours at 26° C. on LB, supplemented with Congo red (CR) (100 mg l⁻¹) to monitor curli assembly. For production of CsgA-fusion proteins, two-layered LB plates were used, with the upper and lower layer containing 0.2% (w/v) glucose and 0.2% (w/v) L-arabinose, respectively.

### Cloning

E. coli DH5α was used for all cloning procedures. To create 6×His-tagged CsgA under the control of the arabinose-inducible P_{BAD} promoter, a DNA fragment encoding the csgA gene, including its signal peptide (amino acids M1 to Y151, accession number P28307), was amplified by PCR with primers CsgA FW 1 and CsgA-HIS Rev 1, using chromosomal DNA of E. coli MC4100 as template. After restriction with Acc65I and XbaI, this fragment was ligated into the pBAD33 vector by standard techniques to create pNA1. The In-Fusion™ PCR cloning kit (ClonTech) was used for fusing the major curli subunit CsgA to the N-terminus of Nb208, a nanobody that recognizes green fluorescent protein (GFP). Nb208, without its signal peptide, was amplified by PCR from plasmid pCA0747 using primers NB208 IF flex FW and NB208 IF Rev. The obtained PCR fragment was inserted into the SmaI linearized pNaA1 plasmid, resulting in pNA15. The same strategy was followed to fuse CsgA to beta-lactamase (amino acids H24 to W286, accession number AAB59737.1, pET22b (Novagen) as template), phosphatase A (amino acids P27 to K471, accession number NP_414917, pGV4220 as template (Pattery et al., 1999)), FimC (amino acids G37 to E241, accession number P31697, K514 (Colson et al., 1965) total DNA as template), FedF (amino acids N35 to K185, accession number CAA81288, pExp62 (Moonens et al., 2012) as template), RNase1 (amino acids L1 to Y245, accession number 2PQX_A (Messens et al., 2007)), mCherry (amino acids V1 to K235, accession number ADV78248, psWU30gltFC (Luciano et al., 2011) as template) and ERD10 (amino acids A2 to D260, accession number AEE29973.1 (Kovacs et al., 2008)). pNA35, a C22S mutant of Nb208 in pNA15, was constructed by site-directed mutagenesis (QuikChange protocol, Stratagene) with primers FW mut ser and Rev mut ser starting from pNA15. pNA48, encoding 6×His-tagged ERD10 with the CsgA N-terminal signal peptide only (CsgAN1-A20), was created by outwards PCR with primers Delta A pNA36 FW and DelN22 Rev on pNA36. Deletions of csgA repeats in the csgA-NB208 fusions were carried out by “outwards” PCR on pNA15; primer combinations are described in Example 6. For cytoplasmic expression, csgA-His was amplified by PCR with primers CsgA FW2 and Csg-His Rev2, and cloned into the NdeI/EcoRI sites of pET22b, resulting in pNA9. Via mutagenesis PCR a BamHI restriction site was introduced between csgA and the His-tag in pNA9 (primers BamHI mut FW and BamHI mut Rev), giving rise to pNA52. Next, a PCR fragment of NB208 without signal peptide (primers NB208 IF petBamHI FW and NB208 IF petBamHI Rev on pNA15 as template) was inserted in the BamHI cut pNA52, resulting in pNA53. The gateway cloning system (Invitrogen) was used to generate the vectors for expression of CsgA fusion proteins in Lactococcus lactis cells. His-tagged csgA fusions were amplified from pNA15 and pNA31 to first generate “Entry” clones by BP recombination using primers CsgA-1 and CsgA-2. These Entry vectors were subsequently recombined with a Lactococcus shuttle vector, pTRKH3, harboring different promoters (i.e., Cplc, LacA1, P9, SplA, P43) (Mc Cracken et al., 2000, Rud et al., 2006). NVG1, a csgG deletion mutant of LSR10 was made as described (Datsenko and Wanner, 2000) using primers FwpKD3csgG and RevpKD3csgG. Bacterial strains, plasmids and primers utilized in this work are listed in Tables 4 and 5.

### Recombinant Gene Expression

Recombinant gene expression was induced in E. coli DH5α at OD₆₀₀ 0.6, by adding L-arabinose to a final concentration of 0.2% (w/v) and incubating at 37° C. For induction in LSR10 cells, bacteria were grown on two-layered plates (described in bacterial growth conditions) for 48 hours at 26° C. Bacteria were scraped off, resuspended in PBS (pH 7.4) and normalized by optical density at 600 nm. For the in vitro fiber formation experiments, E. coli BL21DE3 (pNA53) cells, grown in LB medium at 37° C. till OD₆₀₀ₙₘ 0.9, were induced with 1 mM isopropyl β-D-1-thiogalactopyranoside (IPTG) for 1 hour at 37° C.

The presence of the fusion proteins in bacterial whole-cell lysates was determined by SDS-PAGE and subsequent Western blotting (Sambrook and Russel, 2001), using a mouse anti-his monoclonal antibody (mAb) (MCA1396, AbD Serotec) as primary and an anti-mouse IgG alkaline phosphatase conjugated (Sigma) as secondary antibody.

### (Immuno)Fluorescence Microscopy

For IF microscopy, bacteria were grown and induced as described above. Cells resuspended in PBS were coated onto poly-L-lysine treated microscope slides (Pallesen et al., 1995). Nonspecific binding was blocked by incubation with 5% (w/v) bovine serum albumin (BSA) for 15 minutes. The slides were subsequently incubated for 1 hour with a 1:400 dilution of an anti-6×His mAb (MCA1396, AbD Serotec), washed with PBS and incubated for 1 hour with a 1:250 dilution of ALEXA FLUOR® 594- or ALEXA FLUOR® 488-labeled goat anti-mouse antibody (Invitrogen). For GFP binding studies, after blocking with BSA, a 45 Ξg ml⁻¹ solution of GFP in PBS was added for 1 hour. Slides were examined by a TE2000-U Nikon microscope with a 100 magnification oil immersion lens.

### Dot Blot

Bacteria were scraped from inducing plates, resuspended in PBS at an OD₆₀₀ₙₘ of 1. Where indicated, lysozyme and EDTA were added to a final concentration of 1% (w/v) before incubation at 100° C. for 10 minutes. Five μl samples were spotted on a nitrocellulose membrane and air dried. Membrane blocking for non-specific binding was carried out with a 10% (w/v) skimmed milk (biorad) solution in PBS for 10 minutes. The accessibility of the fusion proteins was determined using a mouse anti-6×His mAb (MCA1396, AbD Serotec) as primary and an anti-mouse IgG alkaline phosphatase conjugated (Sigma) as secondary antibody.

### Transmission Electron Microscopy (TEM)

Bacterial colonies were scraped from inducing plates, resuspended in PBS and 5 μl samples were absorbed onto formvar-coated copper grids (Agar Scientific) for 2 minutes, washed with deionized water, and negatively stained with 1% (w/v) uranyl acetate for 30 seconds. For immunogold labeling, specimens were blocked with 5% (w/v) BSA in PBS for 10 minutes, afterwards incubated with a 1:100 dilution of an anti-6×His mAb (MCA1396, AbD Serotec) for 30 minutes at RT, washed with PBS and finally incubated for 30 minutes with a 1:100 dilution of an anti-mouse 10 nm gold conjugated antibody (G7652, Sigma). Samples were rinsed with PBS followed by distilled water before negative staining. Alternatively, detection of the 6×His-tag in surface exposed fusion proteins was done using 5 nm Ni-NTA-NANOGOLD® (Nanoprobes). Bacteria absorbed onto the grids were incubated for 10 minutes with 20 μl Ni-NTA-NANOGOLD® solution. After washing three times with PBS, the samples were negatively stained. For TEM on SDS-stable fibers, whole cells scraped from inducing plates were boiled in SDS-sample buffer and loaded on an SDS-PAGE gel. After running, the SDS-stable material was recuperated from the slots, 5 μl was coated on the grids and negatively stained. Bacteria were visualized using a JEM-1400 Transmission Electron Microscope (JEOL).

### ELISA

Bacteria were grown and induced as described above. The cells were scraped off the plates and suspended in PBS at an OD₆₀₀ of 1.0. One hundred μl of this cell suspension was coated on 96-well microtiter plates for 2 hours at 37° C. Wells were blocked for 1 hour at 37° C. with 10% skimmed milk powder (Biorad) in PBS prior to incubation with the primary antibodies, either a 1:500 dilution of mouse anti-His mAb (MCA1396, AbD Serotec), a 1:200 dilution of mouse anti-peptidoglycan mAb (7263-1006, AbD Serotec) or a 1:2000 dilution of anti-E. coli polyclonal antibody (4329-4906, AbD Serotec). Wells were subsequently washed and bound antibodies were detected by incubation with an anti-mouse IgG alkaline phosphatase conjugated (Sigma) or anti-rabbit IgG alkaline phosphatase conjugated (Sigma-Aldrich) secondary antibody. Binding was revealed by p-dinitrophenylphosphatase (p-DNPP) (Sigma) as substrate. Absorbance values were measured at 405 nm. To make a comparison between the different experiments and between the different fusion constructs, values obtained for anti-His and anti-pep were divided by the corresponding values for the anti-E. coli response. Statistics were done with the Mann-Whitney test (p-values of 0.05 or 0.001), using pBAD33 as reference.

### Protease Accessibility Assays

Bacterial cells were resuspended in PBS and incubated for 2 hours at 37° C. with 50 Ξg ml⁻¹ Proteinase K (Thermo Scientific). AEBSF was added to a final concentration of 1 mM to stop the reaction. After formic acid treatment, cell lysates were subjected to SDS-PAGE and subsequent Western blotting using an anti-6×His mAb (MCA1396, AbD Serotec), or an anti-DsbA antiserum (kindly provided by J. Messens) as primary antibody and an anti-mouse or anti-rabbit secondary antibody, respectively.

### Purification of Curli

Curli were isolated by a protocol modified from Collinson et al. (1991) as described previously (Dueholm et al., 2013). Samples were subjected to formic acid treatment and evaluated in Western blotting using an anti-6×His mAb (MCA1396, AbD Serotec) or a rabbit anti-CsgA antiserum (kindly provided by M. R. Chapman).

### Purification of CsgA-NB208-his for In Vitro Assays

CsgA-NB208-His without Sec signal sequence is expressed in the cytoplasm of E. coli BL21DE3 and purified via a denaturation method (Zhou et al., 2013). The polymerization kinetics of the purified proteins was followed by ThT fluorescence (Zhou et al., 2013).

### Coupling of In Vitro CsgA Fibers on Magnetic Particles

CsgA-6×His was purified as described above and fibers were grown during 3 weeks at room temperature. Fibers were sonicated to obtain nuclei (Zhou et al., 2013), which were coupled onto Sera-mag magnetic carboxylate-modified microparticles (Thermo Scientific) via the direct EDAC procedure. After coupling, two washes were performed with an MES buffer. Pellets were resuspended between those washes by ultrasonication. The final pellet was resuspended again in MES buffer.

### Mass Spectrometry

RNase1 and isolated CsgA-RNase1 curli were digested in solution overnight at 37° C. with sequencing grade-modified trypsin (Promega, Madison, Wis., USA) in 25 mM NH4HCO3. Prior to mass spectrometry analysis, the samples were desalted on ZipTip C18 (Millipore, Billerica, Mass., USA) and eluted in 50% CH3CN/1% HCOOH (v/v). The samples were loaded into gold-palladium-coated borosilicate nanoelectrospray capillaries (Thermo Fisher Scientific, Waltham, Mass., USA) and ESI mass spectra were acquired on a Q-Tof Ultima mass spectrometer (Waters, Milford, Mass., USA), equipped with a Z-spray nanoelectrospray source and operating in the positive ion mode. Capillary voltages of 1.5-1.8 kV and cone voltage of 50 V typically were used. The source temperature was held at 80° C. Data acquisition was performed using the MassLynx 4.1 software. The spectra represent the combination of 1 second scans. The tryptic peptides were initially identified by peptide mass fingerprinting (PMF). The identity of predicted disulphide-bound peptides was confirmed by tandem mass spectrometry (MS/MS). After processing of the MS/MS data by the maximum entropy data enhancement program MaxEnt 3, the amino acid sequences were semi-automatically determined using the peptide sequencing program PepSeq (Waters, Milford, Mass., USA).

TABLE 1
Examples of polypeptide sequences

| | SEQ | |
|---|---|---|
| Protein/subunit | ID NO | AA sequence |
| CsgA | 1 | MKLLKVAAIAAIVFS |
| (amino acids M1 to Y151, | | GSALAGVVPQYGGGG |
| accession number P28307) | | NHGGGGNNSGPNSEL |
| | | NIYQYGGGNSALALQ |
| | | TDARNSDLTITQHGG |
| | | GNGADVGQGSDDSSI |
| | | DLTQRGFGNSATLDQ |
| | | WNGKNSEMTVKQFGG |
| | | GNGAAVDQTASNSSV |
| | | NVTQVGFGNNATAHQ |
| | | Y |
| CsgA fragment | 2 | GVVPQYGGGGNHGGG |
| (amino acids G21 to Y151, | | GNNSGPNSELNIYQY |
| accession number P28307, | | GGGNSALALQTDARN |
| without signal peptide) | | SDLTITQHGGGNGAD |
| | | VGQGSDDSSIDLTQR |
| | | GFGNSATLDQWNGKN |
| | | SEMTVKQFGGGNGAA |
| | | VDQTASNSSVNVTQV |
| | | GFGNNATAHQY |
| CsgA fragment | 3 | SELNIYQYGGGNSAL |
| (amino acids S43 to Y151, | | ALQTDARNSDLTITQ |
| accession number P28307, | | HGGGNGADVGQGSDD |
| without signal peptide, | | SSIDLTQRGFGNSAT |
| without N22) | | LDQWNGKNSEMTVKQ |
| | | FGGGNGAAVDQTASN |
| | | SSVNVTQVGFGNNAT |
| | | AHQY |
| CsgA fragment | 4 | SELNIYQYGGGNSAL |
| (amino acids S43 to N65, | | ALQTDARN |
| accession number P28307, | | |
| repeat 1 (R1)) | | |
| CsgA fragment | 5 | SDLTITQHGGGNGAD |
| (amino acids S66 to D87, | | VGQGSDD |
| accession number P28307, | | |
| repeat 2 (R2)) | | |
| CsgA fragment | 6 | SSIDLTQRGFGNSAT |
| (amino acids S88 to N110, | | LDQWNGKN |
| accession number P28307, | | |
| repeat 3 (R3)) | | |
| CsgA fragment | 7 | SEMTVKQFGGGNGAA |
| (amino acids S111 to N132, | | VDQTASN |
| accession number P28307, | | |
| repeat 4 (R4)) | | |
| CsgA fragment | 8 | SSVNVTQVGFGNNAT |
| (amino acids S133 to Y151, | | AHQY |
| accession number P28307, | | |
| repeat 5 (R5)) | | |

TABLE 2
Examples of polypeptide sequences

| | SEQ | |
|---|---|---|
| Protein/subunit | ID NO | AA sequence |
| CsgB | 24 | MKNKLLFMMLTILGAPGIAAAA |
| (accession | | GYDLANSEYNFAVNELSKSSFN |
| number | | QAAIIGQAGTNNSAQLRQGGSK |
| P0ABK7) | | LLAVVAQEGSSNRAKIDQTGDY |
| | | NLAYIDQAGSANDASISQGAYG |
| | | NTAMIIQKGSGNKANITQYGTQ |
| | | KTAIVVQRQSQMAIRVTQR |
| CsgD | 25 | MFNEVHSIHGHTLLLITKSSLQ |
| (accession | | ATALLQHLKQSLAITGKLHNIQ |
| number | | RSLDDISSGSIILLDMMEADKK |
| P52106) | | LIHYWQDTLSRKNNNIKILLLN |
| | | TPEDYPYRDIENWPHINGVFYS |
| | | MEDQERVVNGLQGVLRGECYFT |
| | | QKLASYLITHSGNYRYNSTESA |
| | | LLTHREKEILNKLRIGASNNEI |
| | | ARSLFISENTVKTHLYNLFKKI |
| | | AVKNRTQAVSWANDNLRR |
| CsgE | 26 | MKRYLRWIVAAEFLFAAGNLHA |
| (accession | | VEVEVPGLLTDHTVSSIGHDFY |
| number | | RAFSDKWESDYTGNLTINERPS |
| P0AE95) | | ARWGSWITITVNQDVIFQTFLF |
| | | PLKRDFEKTVVFALIQTEEALN |
| | | RRQINQALLSTGDLAHDEF |
| CsgF | 27 | MRVKHAVVLLMLISPLSWAGTM |
| (accession | | TFQFRNPNFGGNPNNGAFLLNS |
| number | | AQAQNSYKDPSYNDDFGIETPS |
| P0AE98) | | ALDNFTQAIQSQILGGLLSNIN |
| | | TGKPGRMVTNDYIVDIANRDGQ |
| | | LQLNVTDRKTGQTSTIQVSGLQ |
| | | NNSTDF |
| CsgG | 28 | MQRLFLLVAVMLLSGCLTAPPK |
| (accession | | EAARPTLMPRAQSYKDLTHLPA |
| number | | PTGKIFVSVYNIQDETGQFKPY |
| P0AEA2) | | PASNFSTAVPQSATAMLVTALK |
| | | DSRWFIPLERQGLQNLLNERKI |
| | | IRAAQENGTVAINNRIPLQSLT |
| | | AANIMVEGSIIGYESNVKSGGV |
| | | GARYFGIGADTQYQLDQIAVNL |
| | | RVVNVSTGEILSSVNTSKTILS |
| | | YEVQAGVFRFIDYQRLLEGEVG |
| | | YTSNEPVMLCLMSAIETGVIFL |
| | | INDGIDRGLWDLQNKAERQNDI |
| | | LVKYRHMSVPPES |

TABLE 3
Polypeptide sequences

| | SEQ | |
|---|---|---|
| | ID NO | AA sequence |
| Nb208 | 13 | QVQLQESGGGLVQAGGSLRLSC |
| | | VASGGTDSNYYMGWFRQAPGKE |
| | | REIVAAISWIGVIERYTDSVKG |
| | | RFTISRENAKNTVALQMNSLNP |
| | | EDTAVYYCAAGRNNRGYSNSWS |
| | | RVASYDYWGQGTQVTVSSGR |
| beta-lactamase | 14 | QVQLQESGGGLVQAGGSLRLSC |
| (amino acids | | VASGGTDSNYYMGWFRQAPGKE |
| H24 to W286, | | REIVAAISWIGVIERYTDSVKG |
| accession number | | RFTISRENAKNTVALQMNSLNP |
| AAB59737.1) | | EDTAVYYCAAGRNNRGYSNSWS |
| | | RVASYDYWGQGTQVTVSSGR |
| phosphatase A | 15 | PVLENRAAQGDITAPGGARRLT |
| (amino acids | | GDQTAALRDSLSDKPAKNIILL |
| P27 to K471, | | IGDGMGDSEITAARNYAEGAGG |
| accession number | | FFKGIDALPLTGQYTHYALNKK |
| NP_414917) | | TGKPDYVTDSAASATAWSTGVK |
| | | TYNGALGVDIHEKDHPTILEMA |
| | | KAAGLATGNVSTAELQDATPAA |
| | | LVAHVTSRKCYGPSATSEKCPG |
| | | NALEKGGKGSITEQLLNARADV |
| | | TLGGGAKTFAETATAGEWQGKT |
| | | LREQAQARGYQLVSDAASLNSV |
| | | TEANQQKPLLGLFADGNMPVRW |
| | | LGPKATYHGNIDKPAVTCTPNP |
| | | QRNDSVPTLAQMTDKAIELLSK |
| | | NEKGFFLQVEGASIDKQDHAAN |
| | | PCGQIGETVDLDEAVQRALEFA |
| | | KKEGNTLVIVTADHAHASQIVA |
| | | PDTKAPGLTQALNTKDGAVMVM |
| | | SYGNSEEDSQEHTGSQLRIAAY |
| | | GPHAANVVGLTDQTDLFYTMKA |
| | | ALGLK |
| FimC | 16 | GVALGATRVIYPAGQKQEQLAV |
| (amino acids | | TNNDENSTYLIQSWVENADGVK |
| G37 to E241, | | DGRFIVTPPLFAMKGKKENTLR |
| accession | | ILDATNNQLPQDRESLFWMNVK |
| number P31697) | | AIPSMDKSKLTENTLQLAIISR |
| | | IKLYYRPAKLALPPDQAAEKLR |
| | | FRRSANSLTLINPTPYYLTVTE |
| | | LNAGTRVLENALVPPMGESTVK |
| | | LPSDAGSNITYRTINDYGALTP |
| | | KMTGVME |
| FedF | 17 | NSSASSAQVTGTLLGTGKTNTT |
| (amino acids | | QMPALYTWQHQIYNVNFIPSSS |
| N35 to K185, | | GTLTCQAGTILVWKNGRETQYA |
| accession number | | LECRVSIHHSSGSINESQWGQQ |
| CAA81288) | | SQVGFGTACGNKKCRFTGFEIS |
| | | LRIPPNAQTYPLSSGDLKGSFS |
| | | LTNKEVNWSASIYVPAIAK |
| RNase1 | 18 | LALQAKQYGDFDRYVLALSWQT |
| (amino acids | | GFCQSQHDRNRNERDECRLQTE |
| L1 to Y245, | | TTNKADFLTVHGLWPGLPKSVA |
| accession | | ARGVDERRWMRFGCATRPIPNL |
| number 2PQX_A) | | PEARASRMCSSPETGLSLETAA |
| | | KLSEVMPGAGGRSCLERYEYAK |
| | | HGACFGFDPDAYFGTMVRLNQE |
| | | IKESEAGKFLADNYGKTVSRRD |
| | | FDAAFAKSWGKENVKAVKLTCQ |
| | | GNPAYLTEIQISIKADAINAPL |
| | | SANSFLPQPHPGNCGKTFVIDK |
| | | AGY |
| mCherry | 19 | VSKGEEDNMAIIKEFMRFKVHM |
| (amino acids | | EGSVNGHEFEIEGEGEGRPYEG |
| V1 to K235, | | TQTAKLKVTKGGPLPFAWDILS |
| accession number | | PQFMYGSKAYVKHPADIPDYLK |
| ADV78248) | | LSFPEGFKWERVMNFEDGGVVT |
| | | VTQDSSLQDGEFIYKVKLRGTN |
| | | FPSDGPVMQKKTMGWEASSERM |
| | | YPEDGALKGEIKQRLKLKDGGH |
| | | YDAEVKTTYKAKKPVQLPGAYN |
| | | VNIKLDITSHNEDYTIVEQYER |
| | | AEGRHSTGGMDELYK |
| ERD10 | 20 | MAEEYKNTVPEQETPKVATEES |
| (amino acids | | SAPEIKERGMFDFLKKKEEVKP |
| A2 to D260, | | QETTTLASEFEHKTQISEPESF |
| accession number | | VAKHEEEEHKPTLLEQLHQKHE |
| AEE29973.1) | | EEEENKPSLLDKLHRSNSSSSS |
| | | SSDEEGEDGEKKKKEKKKKIVE |
| | | GDHVKTVEEENQGVMDRIKEKF |
| | | PLGEKPGGDDVPVVTTMPAPHS |
| | | VEDHKPEEEEKKGFMDKIKEKL |
| | | PGHSKKPEDSQVVNTTPLVETA |
| | | TPIADIPEEKKGFMDKIKEKLP |
| | | GYHAKTTGEEEKKEKVSD |

TABLE 4
Strains and plasmids

| Strain | Genotype | Reference |
|---|---|---|
| DH5α | fhuA2 Δ(argF-lacZ)U169 phoA glnV44 Φ80 Δ(lacZ)M15 | Meleson et al., |
| | gyrA96 recA1 relA1 endA1 thi-1 hsdR17 | 1968 |
| MC4100 | F⁻ araD139 Δ(argF-lac)U169 rpsL150 relA1 deoC1 rbsR | Casadaban, 1968 |
| | fthD5301 fruA25 λ⁻ | |
| BL21DE3 | fhuA2 [lon] ompT gal (λ DE3) [dcm] ΔhsdS | Studier & Moffatt, |
| | λ DE3 = λ sBamHIo ΔEcoRI-B | 1986 |
| | int::(lacI::PlacUV5::T7 gene1) i21 Δnin5 | |
| S. Typhimurium | wild type Salmonella enterica serovar Typhimurium LT2 | Gulig & Curtiss, |
| χ3000 | strain | III, 1987 |
| LSR10 | MC4100 ΔcsgA | Chapman et al., |
| | | 2002 |
| MD1 | MC1000 ΔdsbA::kan | Vertommen et al., |
| | | 2008 |
| NVG1 | LSR10 ΔcsgG | This study |
| Plasmid | Description | Reference |
| pBAD33 | arabinose-inducible expression vector | Guzman et al., |
| | | 1995 |
| pCA0747 | NB208 in pHEN4, expressing NB208 in the periplasm | |
| pNA1 | CsgA-His in pBAD33 | This study |
| pNA15 | CsgA-flex-Nb208-His in pBAD33 | This study |
| pNA18 | CsgA-flex-cAbLys3-His in pBAD33 | This study |
| pNA29 | CsgA-flex-RNase1-His in pBAD33 | This study |
| pNA30 | CsgA-flex-FimC-His in pBAD33 | This study |
| pNA31 | CsgA-flex-Bla-His in pBAD33 | This study |
| pNA32 | CsgA-flex-FedF-His in pBAD33 | This study |
| pNA33 | CsgA-flex-PhoA-His in pBAD33 | This study |
| pNA34 | CsgA-flex-mCherry-His in pBAD33 | This study |
| pNA35 | CsgA-flex-Nb208^{C22S}-His in pBAD33 | This study |
| pNA36 | CsgA-flex-ERD10-His in pBAD33 | This study |
| pNA48 | ERD10-His in pBAD33, containing the csgA signal | This study |
| | peptide (M1-A20) | |
| pNA20 | csgAΔ1-5-flex Nb208-His in pBAD33 | This study |
| pNA21 | csgAΔ2-5-flex Nb208-His in pBAD33 | This study |
| pNA22 | csgAΔ3-5-flex Nb208-His in pBAD33 | This study |
| pNA23 | csgAΔ4-5-flex Nb208-His in pBAD33 | This study |
| pNA24 | csgAΔ5-flex Nb208-His in pBAD33 | This study |
| pNA25 | csgAΔ1-flex Nb208-His in pBAD33 | This study |
| pNA26 | csgAΔN22-flex Nb208-His in pBAD33 | This study |
| pSB1 | CsgAR1-flex NB208-His in pBAD33 | This study |
| pSB2 | CsgAR2-flex NB208-His in pBAD33 | This study |
| pSB3 | CsgAR3-flex NB208-His in pBAD33 | This study |
| pSB4 | CsgAR4-flex NB208-His in pBAD33 | This study |
| pSB5 | CsgAR5-flex NB208-His in pBAD33 | This study |
| pEXP424 | CsgA-flex-NB208-His under control of the Cplc promoter | This study |
| | in pDEST14-pTRKH3 | |
| pEXP435 | CsgA-flex-Bla-His under control of the P9 promoter in | This study |
| | pDEST14-pTRKH3 | |
| pEXP436 | CsgA-flex-Bla-His under control of the Cplc promoter in | This study |
| | pDEST14-pTRKH3 | |
| pEXP437 | CsgA-flex-Bla-His under control of the LacA1 promoter in | This study |
| | pDEST14-pTRKH3 | |
| pEXP438 | CsgA-flex-Bla-His under control of the SplA1 promoter in | This study |
| | pDEST14-pTRKH3 | |
| pEXP439 | CsgA-flex-Bla-His under control of the P43 promoter in | This study |
| | pDEST14-pTRKH3 | |
| pNA53 | CsgA-flex-Nb208-His without SP in pET22b | This study |

TABLE 5
Primers

| | SEQ | |
|---|---|---|
| Primer | ID NO | Sequence (5′-3′) |
| CsgA FW 1 | 21 | CCCCGGTACCCGTTAATT |
| | | TCCATTCGAC |
| CsgA-HIS Rev 1 | 22 | CCCCTCTAGACTAATGGT |
| | | GATGGTGATGGTGCCCGG |
| | | GGTACTGATGAGCGATCG |
| Nb208 IF flex | 23 | GCTCATCAGTACCCCTCT |
| FW | | GGTTCTGGTTCTGGTCAG |
| | | GTGCAGCTGCAG |
| Nb208 IF Rev | 33 | ATGGTGATGGTGCCCGCT |
| | | GGAGACGGTGAC |
| RNase 1 IF flex | 34 | GCTCATCAGTACCCCTCT |
| FW | | GGTTCTGGTTCTGGTTTA |
| | | GCGTTGCAGGC |
| RNase1 IF Rev | 35 | ATGGTGATGGTGCCCATA |
| | | ACCCGCTTTATC |
| PhoA IF flex FW | 36 | GCTCATCAGTACCCCTCT |
| | | GGTTCTGGTTCTGGTCCT |
| | | GTTCTGGAAAAC |
| PhoA IF Rev | 37 | ATGGTGATGGTGCCCTTT |
| | | CAGCCCCAGAGC |
| FedF IF flex FW | 38 | GCTCATCAGTACCCCTCT |
| | | GGTTCTGGTTCTGGTAAT |
| | | TCTAGTGCGAGTAG |
| FedF IF Rev | 39 | ATGGTGATGGTGCCCTTT |
| | | TGCAATCGCAGG |
| Bla IF flex FW | 40 | GCTCATCAGTACCCCTCT |
| | | GGTTCTGGTTCTGGTCAC |
| | | CCAGAAACGCTGG |
| Bla IF Rev | 41 | ATGGTGATGGTGCCCCCA |
| | | ATGCTTAATCAGTG |
| FimC IF flex FW | 42 | GCTCATCAGTACCCCTCT |
| | | GGTTCTGGTTCTGGTGGA |
| | | GTGGCCTTAGGTG |
| FimC IF Rev | 43 | ATGGTGATGGTGCCCTTC |
| | | CATTACGCCCGTC |
| mCherry IF flex | 44 | GCTCATCAGTACCCCTCT |
| FW | | GGTTCTGGTTCTGGTGTG |
| | | AGCAAGGGCGAGG |
| mCherry IF Rev | 45 | ATGGTGATGGTGCCCCTT |
| | | GTACAGCTCGTCC |
| ERD10 IF FW | 46 | GCTCATCAGTACCCCTCT |
| | | GGTTCTGGTTCTGGTGCA |
| | | GAAGAGTACAAGAAC |
| ERD10 IF Rev | 47 | ATGGTGATGGTGCCCATC |
| | | AGACACTTTTTCTTTC |
| FW mut ser | 48 | CTCTCTGAGACTCTCTGC |
| | | TGTAGCCTCTGGAGGC |
| Rev mut ser | 49 | GCCTCCAGAGGCTACAGA |
| | | GGAGAGTCTCAGAGAG |
| Delta A pNA36 | 50 | GCAGAAGAGTACAAGAAC |
| FW | | ACCGTTCCAG |
| DelN22 Rev | 51 | TGCCAGAGCGCTACCGGA |
| | | G |
| FwpKD3csgG | 52 | AATAACTCAACCGATTTT |
| | | TAAGCCCCAGCTTCATAA |
| | | GGAAAATAATCGTGTAGG |
| | | CTGGAGCTGCTTC |
| RevpKD3csgG | 53 | CGCTTAAACAGTAAAATG |
| | | CCGGATGATAATTCCGGC |
| | | TTTTTTATCTGCATATGA |
| | | ATATCCTCCTTAG |
| DelN22FW | 54 | TCTGAGCTGAACATTTAC |
| | | CAGTAC |
| DelN22Rev | 55 | TGCCAGAGCGCTACCGGA |
| | | G |
| DelR1FW | 56 | TCTGACTTGACTATTACC |
| | | CAGC |
| DelR1Rev | 57 | ATTTGGGCCGCTATTATT |
| | | ACCGC |
| Del R5FW | 58 | CCCTCTGGTTCTGGTTCT |
| | | GGTCAGGTG |
| Del R5Rev | 59 | GTTAGATGCAGTCTGGTC |
| | | AAC |
| Del R4-5 Rev | 60 | ATTTTTGCCGTTCCACTG |
| | | ATCAAG |
| Del R3-5 Rev | 61 | GTCATCTGAGCCCTGACC |
| Del R2-5 Rev | 62 | GTTACGGGCATCAGTTTG |
| | | CAG |
| R3 Fw | 63 | AGCTCAATCGATCTGACC |
| | | CAACGTGGCTTCGG |
| R4 Fw | 64 | TCTGAAATGACGGTTAAA |
| | | CAGTTCGGTGG |
| R5 Fw | 65 | TCCTCCGTCAACGTGACT |
| | | CAGGTTGGC |
| CsgA FW2 | 66 | CCCCCATATGGTTGTTCC |
| | | TCAGTACGGCGG |
| Csg-His Rev2 | 67 | CCCCGAATTCCTAATGGT |
| | | GATGGTGAATGGTGGTAC |
| | | TGATGAGCGGTCGCGT |
| BamHI mut FW | 68 | GGTGATGGTGATGGTGGG |
| | | ATCCGTACTGATGAGCGG |
| | | TC |
| BamHI mut Rev | 69 | GACCGCTCATCAGTACGG |
| | | ATCCCACCATCACCATCA |
| | | CC |
| NB208 IF | 70 | TCATCAGTACGGATCCTC |
| petBamHI FW | | TGGTTCTGGTTCTGGTCA |
| | | GGTGCAGCTG |
| NB208 IF | 71 | GGTGATGGTGGGATCCGC |
| petBamHI Rev | | TGGAGACGGTGACCTGG |
| CsgA-1 | 72 | GGGGACAAGTTTGTACAA |
| | | AAAAGCAGGCTTGAAAGG |
| | | AGGaataattaATGAAAC |
| | | TTTTAAAAGTAGCAGCAA |
| | | T |
| CsgA-2 | 73 | GGGGACCACTTTGTACAA |
| | | GAAAGCTGGGTACTAATG |
| | | GTGATGGTGATGGTGC |

Acc65I and XbaI sites are underlined, SmaI site is displayed in bold

### REFERENCES

Agterberg, M. and J. Tommassen (1991). Outer-membrane protein PhoE as a carrier for the exposure of foreign antigenic determinants at the bacterial-cell surface. Antonie Van Leeuwenhoek International Journal of General and Molecular Microbiology 59:249-262.
Barnhart, M. M. and M. R. Chapman (2006). Curli biogenesis and function. Annu. Rev. Microbiol. 60:131-147.
Bertani, G. (2004). Lysogeny at mid-twentieth century: P1, P2, and other experimental systems. J. Bacteriol. 186:595-600.
Casadaban, M. J. (1976). Transposition and fusion of the lac genes to selected promoters in Escherichia coli using bacteriophage lambda and Mu. J. Mol. Biol. 104:541-555.
Chapman, M. R., L. S. Robinson, J. S. Pinkner, R. Roth, J. Heuser, M. Hammar, et al. (2002). Role of Escherichia coli curli operons in directing amyloid fiber formation. Science 295:851-855.
Charbit, A., J. C. Boulain, A. Ryter, and M. Hofnung (1986). Probing the topology of a bacterial membrane protein by genetic insertion of a foreign epitope; expression at the cell surface. EMBO J. 5:3029-3037.
Collinson, S. K., L. Emody, K. H. Muller, T. J. Trust, and W. W. Kay (1991). Purification and characterization of thin, aggregative fimbriae from Salmonella enteritidis. J. Bacteriol. 173:4773-4781.
Colson, C., S. W. Glover, N. Symonds, and K. A. Stacey (1965). The location of the genes for host-controlled modification and restriction in Escherichia coli K-12. Genetics 52:1043-1050.
Datsenko, K. A., and B. L. Wanner (2000). One-step inactivation of chromosomal genes in Escherichia coli K-12 using PCR products. Proc. Natl. Acad. Sci. U.S.A. 97:6640-6645.
Desmyter, A., T. R. Transue, M. A. Ghahroudi, M. H. D. Thi, F. Poortmans, R. Hamers, S. Muyldermans, and L. Wyns, (1996). Crystal structure of a camel single-domain V-H antibody fragment in complex with lysozyme. Nature Structural Biology 3:803-811.
Dosztanyi, Z., V. Csizmok, P. Tompa, and I. Simon (2005). IUPred: web server for the prediction of intrinsically unstructured regions of proteins based on estimated energy content. Bioinformatics (Oxford, England), 21(16):3433-4.
Dueholm, M. S., M. T. Sendergaard, M. Nilsson, G. Christiansen, A. Stensballe, M. T. Overgaard, et al. (2013). Expression of Fap amyloids in Pseudomonas aeruginosa, P. fluorescens, and P. putida results in aggregation and increased biofilm formation. Microbiology Open 2:365-382.
Fronzes, R., H. Remaut, and G. Waksman (2008). Architectures and biogenesis of non-flagellar protein appendages in Gram-negative bacteria. Embo. J. 27:2271-2280.
Gulig, P. A., and R. Curtiss, III (1987). Plasmid-associated virulence of Salmonella typhimurium. Infect. Immun. 55:2891-2901.
Guzman, L. M., D. Belin, M. J. Carson, and J. Beckwith (1995). Tight regulation, modulation, and high-level expression by vectors containing the arabinose PBAD promoter. J. Bacteriol. 177:4121-4130.
Hamers-Casterman, C., T. Atarhouch, S. Muyldermans, G. Robinson, C. Hamers, E. G. Songa, N. Bendahman, and R. Hamers (1993). Naturally occurring antibodies devoid of light chains. Nature 363:446-448.
Huang, H., Y. J. Wang, A. P. White, J. Z. Meng, G. R. Liu, S. L. Liu, and Y. D. Wang (2009). Salmonella expressing a T-cell epitope from Sendai virus are able to induce anti-infection immunity. Journal of Medical Microbiology 58:1236-1242.
Klauser, T., J. Pohlner, and T. F. Meyer (1990). Extracellular transport of cholera toxin B subunit using Neisseria Iga protease beta-domain: conformation-dependent outer membrane translocation. Embo. Journal 9:1991-1999.
Klemm, P. and M. A. Schembri (2000). Fimbriae-assisted bacterial surface display of heterologous peptides. Int. J. Med. Microbiol. 290:215-221.
Kovacs, D., E. Kalmar, Z. Torok, and P. Tompa (2008). Chaperone activity of ERD10 and ERD14, two disordered stress-related plant proteins. Plant Physiology 147:381-390.
Lee, S. Y., J. H. Choi, and Z. Xu (2003). Microbial cell-surface display. Trends Biotechnol. 21:45-52.
Luciano, J., R. Agrebi, A. V. Le Gall, M. Wartel, F. Fiegna, A. Ducret, et al. (2011). Emergence and modular evolution of a novel motility machinery in bacteria. Plos Genet. 7:e1002268.
McCracken, A., M. S. Turner, P. Giffard, L. M. Hafner, and P. Timms (2000). Analysis of promoter sequences from Lactobacillus and Lactococcus and their activity in several Lactobacillus species. Arch. Microbiol. 173:383-389.
Meng, J. Z., Y. J. Dong, H. Huang, S. Li, Y. Zhong, S. L. Liu, and Y. D. Wang (2010). Oral vaccination with attenuated Salmonella enterica strains encoding T-cell epitopes from tumor antigen NY-ESO-1 induces specific cytotoxic T-lymphocyte responses. Clinical and Vaccine Immunology 17:889-894.
Meselson, M. and R. Yuan (1968). DNA restriction enzyme from E. coli. Nature 217:1110-1114.
Messens, J. and J. F. Collet (2006). Pathways of disulfide bond formation in Escherichia coli. International Journal of Biochemistry and Cell Biology 38:1050-1062.
Messens, J., J. F. Collet, K. Van Belle, E. Brosens, R. Loris, and L. Wyns (2007). The oxidase DsbA folds a protein with a nonconsecutive disulfide. J. Biol. Chem. 282:31302-31307.
Moonens, K., J. Bouckaert, A. Coddens, T. Tran, S. Panjikar, M. De Kerpel, et al. (2012). Structural insight in histo-blood group binding by the F18 fimbrial adhesin FedF. Mol. Microbiol. 86:82-95.
Nakamoto, H. and J. C. A. Bardwell (2004). Catalysis of disulfide bond formation and isomenization in the Escherichia coli peniplasm. Biochimica et Biophysica Acta-Molecular Cell Research, 1694:111-119.
Olsen, A., A. Jonsson, and S. Normark (1989). Fibronectin binding mediated by a novel class of surface organelles on Escherichia coli. Nature 338:652-655.
Pallesen, L., L. K. Poulsen, G. Christiansen, and P. Klemm (1995). Chimeric FimH adhesin of type 1 fimbriae: a bacterial surface display system for heterologous sequences. Microbiology 141 (Pt 11):2839-2848.
Pattery, T., J. P. Hemalsteens, and H. De Greve (1999). Identification and molecular characterization of a novel Salmonella enteritidis pathogenicity islet encoding an ABC transporter. Mol. Microbiol. 33:791-805.
Robinson, L. S., E. M. Ashman, S. J. Hultgren, and M. R. Chapman (2006). Secretion of curli fibre subunits is mediated by the outer membrane-localized CsgG protein. Mol. Microbiol. 59:870-881.
Rud, I., P. R. Jensen, K. Naterstad, and L. Axelsson (2006). A synthetic promoter library for constitutive gene expression in Lactobacillus plantarum. Microbiol. 152:1011-1019.
Ruppert, A., N. Arnold, and G. Hobom (1994). OmpA-FMDV VP1 fusion proteins: production, cell-surface exposure and immune responses to the major antigenic domain of foot-and-mouth disease virus. Vaccine 12:492-498.
Sambrook, J. and D. W. Russell (2001). Molecular Cloning: a Laboratory Manual, 3rd edn. Cold Spring Harbor, N.Y.: Cold Spring Harbor Laboratory.
Samuelson, P., E. Gunneriusson, P. A. Nygren, and S. Stahl (2002). Display of proteins on bacteria. J. Biotechnol. 96:129-154.
Studier, F. W., and B. A. Moffatt (1986). Use of bacteriophage T7 RNA polymerase to direct selective high-level expression of cloned genes. J. Mol. Biol. 189(1):113-130.
Van Gerven, N., G. Waksman, and H. Remaut (2011). Pili and Flagella: Biology, Structure, and Biotechnological Applications. Progress in Molecular Biology and Translational Science, Vol 103: Molecular Assembly in Natural and Engineered Systems, 103:21-72.
Veiga, E., V. de Lorenzo, and L. A. Fernández (1999). Probing secretion and translocation of a beta-autotransporter using a reporter single-chain Fv as a cognate passenger domain. Mol. Microbiol. 33:1232-1243.
Vertommen, D. et al. (2008). The disulphide isomerase DsbC cooperates with the oxidase DsbA in a DsbD-independent manner. Mol. Micro. 67:336-349.
Wang, X. and M. R. Chapman (2008). Sequence determinants of bacterial amyloid formation. J. Mol. Biol. 380:570-580.
Wernerus, H. and S. Stahl (2004). Biotechnological applications for surface-engineered bacteria. Biotechnol. Appl. Biochem. 40:209-228.
White, A. P., S. K. Collinson, J. Burian, S. C. Clouthier, P. A. Banser, and W. W. Kay (1999). High efficiency gene replacement in Salmonella enteritidis: chimeric fimbrins containing a T-cell epitope from Leishmania major. Vaccine 17:2150-2161.
White, A. P., S. K. Collinson, P. A. Banser, D. J. Dolhaine, and W. W. Kay (2000). Salmonella enteritidis fimbriae displaying a heterologous epitope reveal a uniquely flexible structure and assembly mechanism. J. Mol. Biol. 296:361-372.
Zhou, Y., D. R. Smith, D. A. Hufnagel, and M. R. Chapman (2013). Experimental Manipulation of the Microbial Functional Amyloid Called Curli. Methods Mol. Biol. 966:53-75.

1. A method of producing a functionalized fiber, the method comprising:
culturing a host cell that is genetically engineered to express a chimeric polypeptide comprising:
a carrier polypeptide comprising the peptide V/I/L-X-Q-X-G-X-X-N/Q-X-A/V/I/L-X-X-X-Q (SEQ ID NO: 32) wherein X is independently any amino acid,
a passenger polypeptide of 50 amino acids or more, and
optionally, a linker that couples the carrier polypeptide to the passenger polypeptide
under suitable conditions to express the chimeric polypeptide, and
allowing the chimeric polypeptide to polymerize into a fiber, wherein the passenger polypeptide is displayed as a functionally active polypeptide.

2. The method of claim 1, wherein the polymerization step occurs on or near the extracellular surface of the same or another host cell.

3. The method of claim 1, wherein:
the polymerization step occurs on or near an artificial surface, or
the polymerization step occurs in solution.

4. The method of claim 1, wherein the expressed chimeric polypeptide is secreted.

5. The method of claim 1, further comprising:
isolating the expressed chimeric polypeptide from the cell before the polymerization step.

6. The method of claim 1, wherein the passenger polypeptide of the chimeric polypeptide is maintained as a functionally active polypeptide after secretion or isolation.

7. The method of claim 1, wherein the host cell is a bacterial host cell.

8. The method of claim 1, wherein the host cell expresses, either endogenously or exogenously, polynucleotide encoding CsgG, and at least one polynucleotide encoding one or more of CsgB, CsgC, CsgE, CsgF, or variants or fragments of any thereof.

9. The method of claim 1, wherein the carrier polypeptide of the chimeric polypeptide has the following structure: (Y₂ᵢ₋₁-Xᵢ-Y₂ᵢ)ₙ, wherein:
n is an integer from 1 to 20 and i increases from 1 to n with each repeat;
each Xᵢ corresponds to the peptide V/I/L-X-Q-X-G-X-X-N/Q-X-A/VI/L-X-X-X-Q (SEQ ID NO: 32) wherein X is independently any amino acid; and
each Y₂ᵢ₋₁ and Y₂ᵢ are independently selected from 0 to 20 contiguous amino acids, wherein the total length of each Y₂ᵢ₋₁-Xᵢ-Y₂ᵢ is not more than 50 amino acids.

10. The method of claim 9, wherein n is 1.

11. The method of claim 1, wherein the carrier polypeptide of the chimeric polypeptide is selected from the group consisting of:
a polypeptide having the peptide of SEQ ID NO: 3;
a polypeptide that has at least 60% amino acid identity with SEQ ID NO: 3;
a fragment of a polypeptide having the peptide of SEQ ID NO: 3 or a fragment of a polypeptide that has at least 60% amino acid identity with SEQ ID NO: 3;
a polypeptide having the peptide of SEQ ID NOS: 4-8; and
a polypeptide that has at least 60% amino acid identity with SEQ ID NOS; 4-8.

12. The method of claim 1, wherein the chimeric polypeptide further comprises a signal peptide.

13. The method of claim 1, wherein the passenger polypeptide comprised in the chimeric polypeptide is between 100 amino acids and 250 amino acids.

14. A functionalized fiber obtained by the method according to claim 1.

15. A recombinant nucleic acid molecule comprising a polynucleotide encoding a chimeric polypeptide, the chimeric polypeptide comprising:
a carrier polypeptide comprising the peptide V/I/L-X-Q-X-G-X-X-N/Q-X-A/V/I/L-X-X-X-Q (SEQ ID NO: 32) wherein X is independently any amino acid,
a passenger polypeptide of at least 50 amino acids, and
optionally, a linker that couples the carrier polypeptide to the passenger polypeptide.

16. The recombinant nucleic acid molecule of claim 15, wherein the carrier polypeptide of the chimeric polypeptide has the following structure: (Y₂ᵢ₋₁-Xᵢ-Y₂ᵢ)ₙ, wherein:
n is an integer from 1 to 20 and i increases from 1 to n with each repeat;
each Xᵢ corresponds to the peptide VII/L-X-Q-X-G-X-X-N/Q-X-A/V/I/L-X-X-X-Q (SEQ ID NO: 32) wherein X is independently any amino acid; and
each Y₂ᵢ₋₁ and Y₂ᵢ are independently selected from 0 to 20 contiguous amino acids, wherein the total length of each Y₂ᵢ₋₁-Xᵢ-Y₂ᵢ is not more than 50 amino acids.

17. The recombinant nucleic acid molecule of claim 16, wherein n is 1.

18. The recombinant nucleic acid molecule of claim 15, wherein the carrier polypeptide of the chimeric polypeptide is selected from the group consisting of:
the polypeptide of SEQ ID NO: 3,
a polypeptide that has at least 60% amino acid identity with SEQ ID NO: 3,
a fragment of a polypeptide having the polypeptide of SEQ ID NO: 3 or a fragment of a polypeptide that has at least 60% amino acid identity with SEQ ID NO: 3,
the polypeptide of SEQ ID NOS: 4-8, and
a polypeptide that has at least 60% amino acid identity with SEQ ID NOS: 4-8.

19. The recombinant nucleic acid molecule of claim 15, wherein the chimeric polypeptide further comprises a signal peptide.

20. The recombinant nucleic acid molecule of claim 15, wherein the passenger polypeptide comprised in the chimeric polypeptide is an enzyme or a binding domain.

21. A vector comprising the recombinant nucleic acid molecule of claim 15.

22. A host cell comprising the recombinant nucleic acid molecule of claim 15.

23. The host cell of claim 22, which is a bacterial host cell.

24. The host cell of claim 22, wherein the host cell is genetically engineered to express, either endogenously or exogenously, a polynucleotide encoding CsgG, and at least one polynucleotide encoding one or more of CsgB, CsgC, CsgE, CsgF, or variants or fragments of any thereof.

25. The host cell of claim 22, which is a component of a bacterial biofilm.

26. A chimeric polypeptide encoded by the recombinant nucleic acid molecule of claim 15.

27. A composition comprising one or more chimeric polypeptides encoded by the recombinant nucleic acid molecule of claim 15, wherein the passenger polypeptide of each chimeric polypeptide in the composition is a functionally active polypeptide.

28. The composition of claim 27, which is a fiber composition.

29. The composition of claim 28, which is attached to a surface.

30. A method of detecting and/or capturing a substance, wherein the substance is selected from the group consisting of a protein, an organic compound, an inorganic compound, a heavy metal, and a pollutant, the method comprising:
utilizing the composition of claim 27 for detecting and/or capturing of the substance.

31. A method of chemically or enzymatically converting a substance, wherein the substance is selected from the group consisting of a protein, an organic compound, an inorganic compound, a heavy metal, and a pollutant, the method comprising:
utilizing the composition of claim 27 for the chemical and/or enzymatic conversion of the substance.

32. A method for producing a chimeric polypeptide in the extracellular medium of a host cell culture, the method comprising:
culturing a host cell that is genetically engineered to express a CsgG protein, or variant or fragment thereof, and a chimeric polypeptide comprising:
a carrier polypeptide comprising the peptide V/I/L-X-Q-X-G-X-X-N/Q-X-A/V/I/L-X-X-X-Q (SEQ ID NO: 32) wherein X is independently any amino acid,
a passenger polypeptide of 50 amino acids or more, and
optionally, a linker that couples the carrier polypeptide to the passenger polypeptide
under suitable conditions to express and secrete the chimeric polypeptide into the extracellular medium,
wherein the CsgG protein, or variant or fragment thereof, and the chimeric polypeptide are expressed concomitantly, and wherein the passenger polypeptide of the chimeric polypeptide is maintained as an active polypeptide after secretion.

33. The method of claim 32, wherein the host cell is genetically engineered to simultaneously express CsgE, or a variant or a fragment thereof.

34. The method of claim 33, further comprising:
isolating the chimeric polypeptide from the culture medium.

35. The method according to claim 7, wherein the host cell is a Gram-negative bacterial host cell.

36. The host cell of claim 23, wherein the host cell is a Gram-positive bacterial host cell.

37. The composition of claim 29, wherein the surface is a cell surface or an artificial surface.


SEQUENCE LISTING (77 sequences)

SEQ ID NO: 1 - protein, 151 aa, Escherichia coli
MKLLKVAAIAAIVFSGSALAGVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 2 - protein, 131 aa, Escherichia coli
GVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 3 - protein, 109 aa, Escherichia coli
SELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 4 - protein, 23 aa, Escherichia coli
SELNIYQYGGGNSALALQTDARN

SEQ ID NO: 5 - protein, 22 aa, Escherichia coli
SDLTITQHGGGNGADVGQGSDD

SEQ ID NO: 6 - protein, 23 aa, Escherichia coli
SSIDLTQRGFGNSATLDQWNGKN

SEQ ID NO: 7 - protein, 22 aa, Escherichia coli
SEMTVKQFGGGNGAAVDQTASN

SEQ ID NO: 8 - protein, 19 aa, Escherichia coli
SSVNVTQVGFGNNATAHQY

SEQ ID NO: 9 - protein, 45 aa, Escherichia coli
SELNIYQYGGGNSALALQTDARNSDLTITQHGGGNGADVGQGSDD

SEQ ID NO: 10 - protein, 68 aa, Escherichia coli
SELNIYQYGGGNSALALQTDARNSDLTITQHGGGNGADVGQGSDDSSIDLTQRGFGNSATLDQWNGKN

SEQ ID NO: 11 - protein, 90 aa, Escherichia coli
SELNIYQYGGGNSALALQTDARNSDLTITQHGGGNGADVGQGSDDSSIDLTQRGFGNSATLDQWNGKNSEMTVKQFGGGNGAAVDQTASN

SEQ ID NO: 12 - protein, 86 aa, Escherichia coli
SDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 13 - protein, 130 aa, Lama glama
QVQLQESGGGLVQAGGSLRLSCVASGGTDSNYYMGWFRQAPGKEREIVAAISWIGVIERYTDSVKGRFTISRENAKNTVALQMNSLNPEDTAVYYCAAGRNNRGYSNSWSRVASYDYWGQGTQVTVSSGR

SEQ ID NO: 14 - protein, 130 aa, Artificial Sequence; identical to SEQ ID NO: 13
  beta-lactamase

SEQ ID NO: 15 - protein, 445 aa, Escherichia coli
PVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 16 - protein, 205 aa, Escherichia coli
GVALGATRVIYPAGQKQEQLAVTNNDENSTYLIQSWVENADGVKDGRFIVTPPLFAMKGKKENTLRILDATNNQLPQDRESLFWMNVKAIPSMDKSKLTENTLQLAIISRIKLYYRPAKLALPPDQAAEKLRFRRSANSLTLINPTPYYLTVTELNAGTRVLENALVPPMGESTVKLPSDAGSNITYRTINDYGALTPKMTGVME

SEQ ID NO: 17 - protein, 151 aa, Escherichia coli
NSSASSAQVTGTLLGTGKTNTTQMPALYTWQHQIYNVNFIPSSSGTLTCQAGTILVWKNGRETQYALECRVSIHHSSGSINESQWGQQSQVGFGTACGNKKCRFTGFEISLRIPPNAQTYPLSSGDLKGSFSLTNKEVNWSASIYVPAIAK

SEQ ID NO: 18 - protein, 245 aa, Escherichia coli
LALQAKQYGDFDRYVLALSWQTGFCQSQHDRNRNERDECRLQTETTNKADFLTVHGLWPGLPKSVAARGVDERRWMRFGCATRPIPNLPEARASRMCSSPETGLSLETAAKLSEVMPGAGGRSCLERYEYAKHGACFGFDPDAYFGTMVRLNQEIKESEAGKFLADNYGKTVSRRDFDAAFAKSWGKENVKAVKLTCQGNPAYLTEIQISIKADAINAPLSANSFLPQPHPGNCGKTFVIDKAGY

SEQ ID NO: 19 - protein, 235 aa, Artificial Sequence
  mCherry
VSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 20 - protein, 260 aa, Arabidopsis thaliana
MAEEYKNTVPEQETPKVATEESSAPEIKERGMFDFLKKKEEVKPQETTTLASEFEHKTQISEPESFVAKHEEEEHKPTLLEQLHQKHEEEEENKPSLLDKLHRSNSSSSSSSDEEGEDGEKKKKEKKKKIVEGDHVKTVEEENQGVMDRIKEKFPLGEKPGGDDVPVVTTMPAPHSVEDHKPEEEEKKGFMDKIKEKLPGHSKKPEDSQVVNTTPLVETATPIADIPEEKKGFMDKIKEKLPGYHAKTTGEEEKKEKVSD

SEQ ID NO: 21 - DNA, 28 bp, Artificial Sequence
  Primer
ccccggtacccgttaatttccattcgac

SEQ ID NO: 22 - DNA, 54 bp, Artificial Sequence
  Primer
cccctctagactaatggtgatggtgatggtgcccggggtactgatgagcgatcg

SEQ ID NO: 23 - DNA, 48 bp, Artificial Sequence
  Primer
gctcatcagtacccctctggttctggttctggtcaggtgcagctgcag

SEQ ID NO: 24 - protein, 151 aa, Escherichia coli
MKNKLLFMMLTILGAPGIAAAAGYDLANSEYNFAVNELSKSSFNQAAIIGQAGTNNSAQLRQGGSKLLAVVAQEGSSNRAKIDQTGDYNLAYIDQAGSANDASISQGAYGNTAMIIQKGSGNKANITQYGTQKTAIVVQRQSQMAIRVTQR

SEQ ID NO: 25 - protein, 216 aa, Escherichia coli
MFNEVHSIHGHTLLLITKSSLQATALLQHLKQSLAITGKLHNIQRSLDDISSGSIILLDMMEADKKLIHYWQDTLSRKNNNIKILLLNTPEDYPYRDIENWPHINGVFYSMEDQERVVNGLQGVLRGECYFTQKLASYLITHSGNYRYNSTESALLTHREKEILNKLRIGASNNEIARSLFISENTVKTHLYNLFKKIAVKNRTQAVSWANDNLRR

SEQ ID NO: 26 - protein, 129 aa, Escherichia coli
MKRYLRWIVAAEFLFAAGNLHAVEVEVPGLLTDHTVSSIGHDFYRAFSDKWESDYTGNLTINERPSARWGSWITITVNQDVIFQTFLFPLKRDFEKTVVFALIQTEEALNRRQINQALLSTGDLAHDEF

SEQ ID NO: 27 - protein, 138 aa, Escherichia coli
MRVKHAVVLLMLISPLSWAGTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 28 - protein, 277 aa, Escherichia coli
MQRLFLLVAVMLLSGCLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 29 - protein, 18 aa, Escherichia coli
  misc_feature (2)..(6): Xaa can be any naturally occurring amino acid
SXXXXXQXXXXNXXXXXQ

SEQ ID NO: 30 - protein, 20 aa, Escherichia coli
MKLLKVAAIAAIVFSGSALA

SEQ ID NO: 31 - protein, 22 aa, Escherichia coli
GVVPQYGGGGNHGGGGNNSGPN

SEQ ID NO: 32 - protein, 14 aa, Artificial Sequence
  Carrier polypeptide
XXQXGXXXXXXXXQ

SEQ ID NO: 33 - DNA, 30 bp, artificial
  Primer
atggtgatggtgcccgctggagacggtgac

SEQ ID NO: 34 - DNA, 47 bp, artificial
  Primer
gctcatcagtacccctctggttctggttctggtttagcgttgcaggc

SEQ ID NO: 35 - DNA, 30 bp, artificial
  Primer
atggtgatggtgcccataacccgctttatc

SEQ ID NO: 36 - DNA, 48 bp, artificial
  Primer
gctcatcagtacccctctggttctggttctggtcctgttctggaaaac

SEQ ID NO: 37 - DNA, 30 bp, artificial
  Primer
atggtgatggtgccctttcagccccagagc

SEQ ID NO: 38 - DNA, 50 bp, artificial
  Primer
gctcatcagtacccctctggttctggttctggtaattctagtgcgagtag

SEQ ID NO: 39 - DNA, 30 bp, artificial
  Primer
atggtgatggtgcccttttgcaatcgcagg

SEQ ID NO: 40 - DNA, 49 bp, artificial
  Primer
gctcatcagtacccctctggttctggttctggtcacccagaaacgctgg

SEQ ID NO: 41 - DNA, 32 bp, artificial
  Primer
atggtgatggtgcccccaatgcttaatcagtg

SEQ ID NO: 42 - DNA, 49 bp, artificial
  Primer
gctcatcagtacccctctggttctggttctggtggagtggccttaggtg

SEQ ID NO: 43 - DNA, 31 bp, artificial
  Primer
atggtgatggtgcccttccattacgcccgtc

SEQ ID NO: 44 - DNA, 49 bp, artificial
  Primer
gctcatcagtacccctctggttctggttctggtgtgagcaagggcgagg

SEQ ID NO: 45 - DNA, 31 bp, artificial
  Primer
atggtgatggtgccccttgtacagctcgtcc

SEQ ID NO: 46 - DNA, 51 bp, artificial
  Primer
gctcatcagtacccctctggttctggttctggtgcagaagagtacaagaac

SEQ ID NO: 47 - DNA, 34 bp, artificial
  Primer
atggtgatggtgcccatcagacactttttctttc

SEQ ID NO: 48 - DNA, 34 bp, artificial
  Primer
ctctctgagactctctgctgtagcctctggaggc

SEQ ID NO: 49 - DNA, 34 bp, artificial
  Primer
gcctccagaggctacagaggagagtctcagagag

SEQ ID NO: 50 - DNA, 28 bp, artificial
  Primer
gcagaagagtacaagaacaccgttccag

SEQ ID NO: 51 - DNA, 19 bp, artificial
  Primer
tgccagagcgctaccggag

SEQ ID NO: 52 - DNA, 67 bp, artificial
  Primer
aataactcaaccgatttttaagccccagcttcataaggaaaataatcgtgtaggctggagctgcttc

SEQ ID NO: 53 - DNA, 67 bp, artificial
  Primer
cgcttaaacagtaaaatgccggatgataattccggcttttttatctgcatatgaatatcctccttag

SEQ ID NO: 54 - DNA, 24 bp, artificial
  Primer
tctgagctgaacatttaccagtac

SEQ ID NO: 55 - DNA, 19 bp, artificial
  Primer
tgccagagcgctaccggag

SEQ ID NO: 56 - DNA, 22 bp, artificial
  Primer
tctgacttgactattacccagc

SEQ ID NO: 57 - DNA, 23 bp, artificial
  Primer
atttgggccgctattattaccgc

SEQ ID NO: 58 - DNA, 27 bp, artificial
  Primer
ccctctggttctggttctggtcaggtg

SEQ ID NO: 59 - DNA, 21 bp, artificial
  Primer
gttagatgcagtctggtcaac

SEQ ID NO: 60 - DNA, 24 bp, artificial
  Primer
atttttgccgttccactgatcaag

SEQ ID NO: 61 - DNA, 18 bp, artificial
  Primer
gtcatctgagccctgacc

SEQ ID NO: 62 - DNA, 21 bp, artificial
  Primer
gttacgggcatcagtttgcag

SEQ ID NO: 63 - DNA, 32 bp, artificial
  Primer
agctcaatcgatctgacccaacgtggcttcgg

SEQ ID NO: 64 - DNA, 29 bp, artificial
  Primer
tctgaaatgacggttaaacagttcggtgg

SEQ ID NO: 65 - DNA, 27 bp, artificial
  Primer
tcctccgtcaacgtgactcaggttggc

SEQ ID NO: 66 - DNA, 30 bp, artificial
  Primer
cccccatatggttgttcctcagtacggcgg

SEQ ID NO: 67 - DNA, 52 bp, artificial
  Primer
ccccgaattcctaatggtgatggtgaatggtggtactgatgagcggtcgcgt

SEQ ID NO: 68 - DNA, 38 bp, artificial
  Primer
ggtgatggtgatggtgggatccgtactgatgagcggtc

SEQ ID NO: 69 - DNA, 38 bp, artificial
  Primer
gaccgctcatcagtacggatcccaccatcaccatcacc

SEQ ID NO: 70 - DNA, 46 bp, artificial
  Primer
tcatcagtacggatcctctggttctggttctggtcaggtgcagctg

SEQ ID NO: 71 - DNA, 35 bp, artificial
  Primer
ggtgatggtgggatccgctggagacggtgacctgg

SEQ ID NO: 72 - DNA, 73 bp, artificial
  Primer
ggggacaagtttgtacaaaaaagcaggcttgaaaggaggaataattaatgaaacttttaaaagtagcagcaat

SEQ ID NO: 73 - DNA, 52 bp, artificial
  Primer
ggggaccactttgtacaagaaagctgggtactaatggtgatggtgatggtgc

SEQ ID NO: 74 - protein, 4 aa, Artificial Sequence
  Factor Xa cleavage site
IEGR

SEQ ID NO: 75 - protein, 4 aa, Artificial Sequence
  thrombin cleavage site
LVPR

SEQ ID NO: 76 - protein, 5 aa, Artificial Sequence
  enterokinase cleaving site
DDDDK

SEQ ID NO: 77 - protein, 8 aa, Artificial Sequence
  PreScission cleavage site
LEVLFQGP